SEQUENCE LISTING (23 sequences)

SEQ ID NO: 1 - DNA, 409 bp, synthetic construct
  source 1..409: mol_type = other DNA
aaattaacattgaaagttaaaatgtcttttcaatttccgtaaatcttgaataataatttctaacggattgaaatcaattaagttaatagcgacttacaagatgcaaacggcaagatcctatatgaaacttgaatgtactgatatttccaagtcattacagatctctgcaatcttgcagatgatgattaccgcaattatatttattatgttgatctttgcagctagttataacaatatggttaaccctacaggggatgaaggtatggaaaagaaggaaaagtatattaacggtctggtgatatcttcggccgttattagtacacttttattcattattggtgtttggcatgtatatgttgcgatcaaagccaaaaaatgcattacgacaactccagtaactcgtacttaa

SEQ ID NO: 2 - DNA, 1046 bp, synthetic construct
  source 1..1046: mol_type = other DNA
agtggaatctgcaagcttttgtttctttgcaatagactctattgctgaagttttgagtttacgctttccagtggtggtggtgattttcggagaaggctttttactataggaaaagctgggcttatcgacttcactcacgtcatcagtatcagaagagtcttcacagtataaatctgggtccatatctataaataaaaaaaatgaaacaaattgtatttataaattttgtaaatgtacaagatttgaatatggatgatatttacaacttactaatgacgtgtttccaacattttacgcacatgtacattctacatggctcatttgctagatgctttttccaagatgctaaacgtaaaagggtcaatcatgaagatgatttacctggtatttactttataaagaatattcacaataacataaaatgctacagtatgctcataaatgtaaacgatttcaactttgtatatagtaataaatatacacatgttgattacaaaaatctgagcgttggtaagaattttgatttcaatgcactctcgaaagaactatctttgaacctgagatccttttttagaactaacgctaataaaaagaaatctatagcagtgatcgatttggatgatacaatcataaacaaaaatggtgattttatattggaaccctttgacaaatatttagccacacttaaaaatacatttgatttagttgtgttgtggtctcatggatgccaacaacatgtcaaccatgtattctcaaacactttgaatggttatgcaaaagaatttgatatgattatggttcgtagcagttcagtccaaataagtaacaagggtaagggattcttacttcaactgatgaaccaagcttttggagttattgaatttacatattctgtattgatagatgaccaggcgtacaattataagaatgattatgactgttttatacatgtaccaaaatgtttatcacataaagcatacaataagcaaatgtggaacatgttgtataaactgaaaaatactatagataaaagactaaacttgtaa

SEQ ID NO: 3 - DNA, 704 bp, synthetic construct
  source 1..704: mol_type = other DNA
ctgccatttcaataaagtttaaagtatcataacatataataattgcaaaatttgaaccccataaagagtcaccaatattaaaataattcaaatcttcagtatatggtgttatcattttactattaaaatcataataggttttgatatctagatttcttagctttgtaaaagtgcaatttccatctctatcaccaaagtagatggaaaacgaatccgacattgtaagtcttaatactaaaaattatgatgtacttatttattcaaaatcaataaagaatacactaaatttatttattcataattatctttcaattcctgcacttgaactagaaatccaccccggtagattttattatggtacccaccatacaatgggggcttttataaagagaagtgtcataattaagaagctgcaattttgtgaagactgtataaacaagttattaaaacagtcttacgacctttttaaggtttggtattacccgatagtgaattgcgagactctaactcgtggcttaacacaaaacataccaataagtatacaaacaattttaataactggagcatttacaagtcttgttattggtatatcatacccttctgtatttctattaacatttttatttataattttgttattgttatacaataactttcgtttccaactttttagaagcacttgtattcataacccacaagtatga

SEQ ID NO: 4 - DNA, 1494 bp, synthetic construct
  source 1..1494: mol_type = other DNA
gataattaaatctttatgtttaaaaaaaaccactaataaatttataaactgattatgtatttatgtatttcattatgttgaaaataaaatcttttaaaatgttattttttttaatctttctaacatttttataaaatttgtaatttgtgcgagtaaacctatgtagcagcagagaagagagaaataagtgaagtaaataaaatgtcgggagctgaacatatggaaggtgttgagcgggtgctcagggatttgaataatgaggaaaaattaacactcggactgagtgctgtgggtggggtagttggggcaactaagatgatgaatgaagctgcagagcttagaaggaattatggagatcgcccatatgaaaggcttttggagttgagcaagagaaagtatgagtccacaccaacatcaggatttggtgagcgacttagaccatctatggccgaacagctagataggagcaccaagaggccaaggaaagctaacagattcccagtaaacactcattataacatgcacaagatctgcaagaggacaaacctgacatcctccaagttattaggcttttctggccaggctggacaagacatcccaaaatataacagtgcagtaaccctaccattggaggctctagaattttgggtgggagataacataaatccagaagtagcacactccatgggaagcaaagtattgagcgataaggaatgcagggtaaaatcaatgaaactgaaacttagcaacctgcaagtatatgaagacagacaatatggatctggagataggcttagtgaaactgcaaaggatgtttcttgctttgttattccagtagaatcctatatgggtagcaaatcaaatggaactctttgcaagatgttctcggccaatacatcagcaaacagcaatgtcggagattttattacaacaaaggatatcatgatgggaaaattagttacaaaatcagatgttgatagaatgaatgttaacccagaaatgtacatgtggtcatcagaggatggatccctagagctagaattcaaccaggataacgggttgtggtttaggatgccggagcttagagatggggtataccacttactgccaatggaatccggcattggggctagtgcattagaaacttacactataccagaaagtgatgacgagatcctaagaagtaccaccacaattacaccactatcatcggccattggcagtctgctaattggagtaccatatatcattgatgcctctggaaagcaaaaggactacagggtctcatttatggttgaacaggaaattactttggaatgccgcgctgaatggatgcagagctcttcagcttcagactggaatgcagggatgggtgctactctcacaccaagatctacaaatattacagaatttaggcatatggttggaccatacaatgtacaagcagagcatggaaataggatgaaaatgcatcaaactcatcaagcagtataa

SEQ ID NO: 5 - DNA, 46 bp, synthetic construct
  source 1..46: mol_type = other DNA
tgtcttttcaatttccgtaaatcttgaataataatttctaacggat

SEQ ID NO: 6 - DNA, 46 bp, synthetic construct
  source 1..46: mol_type = other DNA
ggtggtggtgattttcggagaaggctttttactataggaaaagctg

SEQ ID NO: 7 - DNA, 46 bp, synthetic construct
  source 1..46: mol_type = other DNA
atgtttaaaaaaaaccactaataaatttataaactgattatgtatt

SEQ ID NO: 8 - DNA, 46 bp, synthetic construct
  source 1..46: mol_type = other DNA
tatataattaaaagaaatatgaatagtattataatcagagataaca

SEQ ID NO: 9 - DNA, 50 bp, synthetic construct
  source 1..50: mol_type = other DNA
tacttttcacacaatcagatatagcattatactatatgtgggtctagaca

SEQ ID NO: 10 - DNA, 50 bp, synthetic construct
  source 1..50: mol_type = other DNA
ccattttggtactatatacagtggtatactatatccctttaaaggtaaaa

SEQ ID NO: 11 - DNA, 50 bp, synthetic construct
  source 1..50: mol_type = other DNA
agatgatttagaatactgtattaattttttaatgttcatttttataagca

SEQ ID NO: 12 - DNA, 46 bp, synthetic construct; identical to SEQ ID NO: 7
  source 1..46: mol_type = other DNA

SEQ ID NO: 13 - DNA, 50 bp, synthetic construct
  source 1..50: mol_type = other DNA
tggcttttggaatactcaaacaatttaaaaaaaatcacaatagactccaa

SEQ ID NO: 14 - DNA, 50 bp, synthetic construct
  source 1..50: mol_type = other DNA
atttagcatttaataaacgaaacgttttatagtataactatattacaaac

SEQ ID NO: 15 - DNA, 50 bp, synthetic construct
  source 1..50: mol_type = other DNA
gtgttttgttggctttggatctttaaatataaaacgtctagcgtaaaaca

SEQ ID NO: 16 - DNA, 50 bp, synthetic construct
  source 1..50: mol_type = other DNA
atatatttttaattaccattgacctactttagtatagtaaagttagaaat

SEQ ID NO: 17 - protein, 10 aa, synthetic construct
LXXXLXXLXL

SEQ ID NO: 18 - protein, 4 aa, synthetic construct
KDEL

SEQ ID NO: 19 - protein, 15 aa, synthetic construct
GGGGGGGGGGGGGGG

SEQ ID NO: 20 - protein, 15 aa, synthetic construct
GGGGSGGGGSGGGGS

SEQ ID NO: 21 - protein, 5 aa, synthetic construct
TEFCA

SEQ ID NO: 22 - protein, 8 aa, synthetic construct
GPLGIAGQ

SEQ ID NO: 23 - DNA, 2243 bp, synthetic construct
  source 1..2243: mol_type = other DNA
gctgcgaactcatcaactgactttaggggttttgaaccaaatttaatatcggctaaccacttcatttctttttaaataattgaggtttcaccactaagaaatatataattaaaagaaatatgaatagtattataatcagagataacatctcattaaaaaaatctttgtatctctacaagacttaatatactaaagaattcatgaacgtctttatctacttgttggttgtttttattttcattacaattttaataatttattattttatacaaaaacctaaaaagtctaaaaatgttgaagatgtttactcaaagtatttgctgtcagaagatacgccagatacagcatttacatacaaacgaagaagcgatggtaaatatgaaaattattatttgaattttgatacggaaggggatctactaaaaatggtagtttctgattacccagttacaaaaactggtgaagattataagccaaaagaacatggaactttaactgaaaatgaaaatggttttagaataagtggagttgctgcagatttcaaatgtcccgatggttggatttggaatagtaataaaaagacatgtagtctgatacccatttgtggaagtgatgacgagggtaagataaaagggttggatttttactactttaatctagcaaaatccatgggtctcataaagtctgctacaaaataccatgaacggttatatattacttgcctggaagaccagtcttataatataaaaacttgtccagacaatatgctctttaacgagttaccaatccaagatgatactggcgaactaccttgcaagttttatgatatctgtgaaggtatgagaaactacgaatttcatagacaacagatctcggaagatgttgttttggagcctagtcaatattatatgtgctttaacggtattagtaatctgaagcaatgtgaagagggtgcatttttcaatgaagaattgggtggttgcttagaagaaaatcaatgtatgggcaaaccagacggtttcacactaccatcagatttaaattcgtacatcctatgtcaaaatgaacaagagtatagcatatattgcaaagatggtatatatgaagcgctcggccccaatgcactcagttgtagtattgataaatcccagacatatttcaagtattttacaaatgattttattagcatacccattgggctatatgtatacaaaaataatcaaaaaagtgagaggtatgcacaaaccgaaatggtcaaaaaatctatggctctagaaccatcaacttcaagatttttcggtgcggttagaaatgatatgctgtatgacccagtagaataccaaaagtattttataacatacttagataatacaaccctagaagattcggtggaagttgaactggacaagacaaactatcaaatatttaacccaaaagctctagtatctgcatgttattatgttcacaggttaccagctttcgattggaatatatttgaagatgttcctgtgtctgattatgtgaataaagagtcatcgtattactacaagtatgacaaagttataaaacatacaaaagatgatacattttctgaaaatagcatagattacttcttcttttgtacagcacagcatttgtataaaccagttgatccaacagttatagttaccgatgaagcgtctggtatattatgttatgcggattttaaaatgtctagatttggcatatcttcaaattatagtaaagcattcaccgtattaaaatttgcaaatatgggagattctacaattttatactatgttgatccatacgaaaatgctatgattgctgcagcttttgacaattccgttatattatcggattttgtaaatgttacagatgatgggtatacatgtacgcccaagatgtttacatatgatgagttgaaaaatgataagccaaatgagtatttttacataagaatgtcatcgataaaatatgatggagtggttgataacactatgagtagatatgtgttgcctgaaatattattacttgtcgacttaaaagttgctacgcaaatgggtaaattctttacaattttaaatactgaaaacctaaatattgcatctgatttaactgtttttagtactatagtagaagattcatttataccaataactgaacatactgaaaatatatcatattttgaaaacgttttagatttgctttaccaggatcatgtttcaaacggtagttcgtga

(19) United States
(12) Patent Application Publication
(10) Pub. No.: US 2026/0347874 A1
(43) Pub. Date: Nov. 5, 2026

(54) NUDIVIRAL PROMOTERS AND USES THEREOF

(71) Applicant: ARIZONA BOARD OF REGENTS ON BEHALF OF THE UNIVERSITY OF ARIZONA, Tucson, AZ (US)

(72) Inventors: Arun Dhar, Tucson, AZ (US); Hung Mai, Tucson, AZ (US)

(21) Appl. No.: 19/119,025

(22) PCT Filed: Oct. 6, 2023
(86) PCT No.: PCT/US2023/076275

§ 371 Date: Apr. 7, 2025

Related U.S. Application Data
(60) Provisional application No. 63/414,266, filed on Oct. 7, 2022.

(51) Int. Cl. C12N 15/86; A01K 67/67; C07K 14/005

(52) U.S. Cl. CPC C12N 15/86; A01K 67/67; C07K 14/005; A01K 2217/052; A01K 2227/70; C12N 2710/00022; C12N 2710/14043

(57) ABSTRACT

Described in several exemplary embodiments herein are polyhedrin promoter sequences from a shrimp nudivirus, vectors containing the same, and uses thereof.

### CROSS-REFERENCE TO RELATED APPLICATIONS

This application claims the benefit of and priority to co-pending U.S. Provisional Patent Application No. 63/414,266, filed on Oct. 7, 2022, entitled “Nudiviral Promoters and Uses Thereof,” the contents of which is incorporated by reference herein in its entirety.

### SEQUENCE LISTING

This application contains a sequence listing filed in electronic form as an xml file entitled “UAZ-0145WP_ST26.xml”, created on Oct. 4, 2023, and having a size of 27,178 bytes. The content of the sequence listing is incorporated herein in its entirety.

### TECHNICAL FIELD

The subject matter disclosed herein is generally directed to regulatory elements, and more particularly promoters, for driving gene expression, particularly heterologous genes.

### BACKGROUND

Promoters are the primary driver for gene expression in a homologous or heterologous expression system. Availability of a promoter element capable of driving a gene expression in a constitutive or inducive manner is fundamental in understanding the biological function of a protein molecule in a cell as well as leveraging expression for various practical applications. For example, a strong promoter is often needed for driving expression of therapeutic protein in a heterologous expression system, such as a bacterial, a yeast, fungi, and insect cell, a mammalian cell and in plants. Thus, there is a need for various promoters, including strong promoters, for expression of gene products, including proteins, in various cell types.

Citation or identification of any document in this application is not an admission that such a document is available as prior art to the present invention.

### SUMMARY

Described in certain example embodiments herein are nudiviral nucleic acids corresponding to a polyhedrin gene promoter of a nudivirus, a variant thereof, or a derivative thereof. In certain example embodiments, the nudivirus is an alpha nudivirus, betanudivirus, deltanudavirus, gammanudavirus, or an epsilonnudavirus. In certain example embodiments, the nudivirus is a shrimp, fish, or insect nudivirus. In certain example embodiments, the nucleic acid comprises or consists of a sequence corresponding to SEQ ID NO: 5, 6, 7, or 8, optionally SEQ ID NO: 7. In certain example embodiments, the nucleic acid comprises or consists of a sequence that is 80-100% identical to SEQ ID NOs: SEQ ID NO: 5, 6, 7, or 8, optionally SEQ ID NO: 7.

Described in certain example embodiments herein are engineered polynucleotides comprising the nudiviral nucleic acid of the present description; and a polynucleotide encoding a non-polyhedrin polynucleotide and/or polypeptide, wherein the nudiviral nucleic acid is operatively coupled to the polynucleotide encoding a non-polyhedrin polynucleotide.

Described in certain example embodiments herein are vectors or vector systems comprising a nudiviral nucleic acid of the present description, an engineered polynucleotide of the present description, or both. In certain example embodiments, the vector is an expression vector or vector system.

Described in certain example embodiments herein are cell(s) comprising a nudiviral nucleic acid of the present description; an engineered polynucleotide of the present description; a vector or vector system of the present description; or any combination thereof. In certain example embodiments, the cell is a prokaryotic or eukaryotic cell. In certain example embodiments, the cell is a shrimp cell, fish cell, insect cell, or a plant cell. In certain example embodiments, the cell is a mammalian cell, optionally a human cell.

Described in certain example embodiments herein are cell population comprising one or more cells of the present description.

Described in certain example embodiments herein are organisms comprising a nudiviral nucleic acid of the present description; an engineered polynucleotide of the present description; a vector or vector system of the present description; a cell or cell population as in the present description; or any combination thereof. Described in certain example embodiments, the organism is a non-human animal, insect, or a plant. In certain example embodiments herein, the organism is a crustacean or fish.

Described in certain example embodiments herein are formulations comprising a nudiviral nucleic acid of the present description; an engineered polynucleotide of the present description; a vector or vector system of the present description; a cell or cell population as in the present description; or any combination thereof, and a pharmaceutically acceptable carrier.

Described herein are certain example embodiments herein are kits comprising a nudiviral nucleic acid of the present description; an engineered polynucleotide of the present description; a vector or vector system of the present description; a cell or cell population as in the present description; or a formulation of the present description, or any combination thereof.

Described in certain example embodiments herein are methods comprising expressing an engineered polynucleotide of the present description or a vector or vector system as in any one of the present description in vitro, in vivo, or ex vivo.

Described in certain example embodiments herein are methods of expressing an engineered nucleic acid, the method comprising placing an engineered polynucleotide of the present description or a vector or vector system of the present description under condition(s) and/or environment(s) such that the non-polyhedrin polynucleotide is transcribed and optionally translated. In certain example embodiments, wherein expression occurs in vitro, in vivo, or ex vivo. In certain example embodiments, the method further comprises operatively coupling the non-polyhedrin encoding nucleic acid to a nudiviral nucleic acid of the present description.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1A-1C—PvSNPV detection by conventional PCR and wet mount analyses. (FIG. 1A) PvSNPV PCR detection in Penaeus vannamei broodstock. M=DNA ladder; NTC=non-template control; SPF=Specific Pathogen Free (SPF) sample; 1-12=quarantine samples; +VE=positive control. (FIG. 1B) Wet mount examination of fecal samples originating from SPF P. vannamei juvenile experimentally challenged with PvSNPV. Black arrows indicated occlusion bodies. (FIG. 1C) PvSNPV detection in experimentally challenged PvSNPV challenged juvenile. 1-5=PvSNPV challenged samples.

FIG. 2—Circular genomic map of Penaeus vannamei singly enveloped nuclear polyhedrosis virus (PvSNPV). The green boxes indicate tandem repeat regions. Putative promoters are indicated by red. The predicted protein coding genes and their orientation were indicated by yellow arrows. The outer yellow arrows indicate the predicted protein coding genes on forward strand. The inner yellow arrows indicate the predicted protein coding genes on reverse strand. The GC contents are indicated by blue, and the AT contents are indicated by green. The numbers represent the base numbers.

FIG. 3A-3B—A phylogeny analysis of PvSNPV and other nudiviruses. (FIG. 3A) A super matrix phylogenetic tree based on 16 orthologous protein sequences (PIF-2, PIF-0/P74, DNAPOL, AC-92, FEN-1, LEF-8, PIF-1, INTEGRASE, LEF-9, 38K, AC-81 LIKE, PIF-6, LEF-4, PIF-3, ODV-E28, and GB-67 LIKE) from 16 nudiviruses including Penaeus monodon nudivirus (PmNV) (NC_024692), PvSNPV (OM066077), CcNV=Crangon nudivirus (MZ311577), CmNV=Carcinus maenas nudivirus (MZ311578), DhNV=Dikerogammarus haemobaphes nudivirus (MT488302.1), DiNV=Drosophila innubila nudivirus (NC_040699.1), ENV=Esparto virus (NC_040536.1), GbNV=Gryllus bimaculatus nudivirus (NC_009240.1), HgNV=Homarus gammarus nudivirus (MK439999.1), HzNV-1=Heliothis nudivirus 1 (AF451898.1), HzNV-2=Heliothis nudivirus 2 (NC_004156.2), KNV=Kallithea virus (NC_033829.1), MNV=Mauternbach virus (MG969167), OrNV=Oryctes rhinoceros nudivirus (NC_011588.1), TNV=Tomelloso virus (NC_040789.1), ToNV=Tipula oleracea nudivirus (NC_026242.1). (FIG. 3B) A super matrix phylogenetic tree based on 24 core protein sequences (DNA POLYMERASE, AC92-LIKE, VP91, ODV-E56, P47, PIF-2, FEN-1, LEF-8, PIF-1, INTERGRASE, LEF-9, 38K, P74, HELICASE-2, AC81-LIKE, PIF-6, VLF1, LEF-4, PIF-3, HELICASE, PIF-4, ESTERASE, GP-67 LIKE, 11K) from 16 nudiviruses.

FIG. 4—A gene synteny between PmNV (Green) and PvSNPV (Red). The genes on the forward strand were indicated by grey color. The genes on reverse strand were indicated by a blue color. The red ribbons connected core genes between PmNV and PvSNPV. The figure was generated by Circa (https://omgenomics.com/circa/).

FIG. 5A-5B—A promoter analysis of polyhedrin gene. (FIG. 5A (SEQ ID NO: 9-16)) Multiple alignment (MAFFT, select an appropriate strategy according to data size) of putative promoters of polyhedrins from seven nudiviruses predicted by Neural Network Promoter Prediction (NNPP) (type organism: prokaryote; include reverse strand: No; Minimum promoter score: 0.8) (Reese, 2001). The multiple alignment was done by Clustaw2 alignment. The transcription start site (TSS) was indicated by yellow color. (FIG. 5B) The sequence logo of polyhedrin alignment was generated by the WebLoGo (http://weblogo.berkeley.edu/). The higher letters indicated higher level of consensus.

The figures herein are for illustrative purposes only and are not necessarily drawn to scale.

### DETAILED DESCRIPTION OF THE EXAMPLE EMBODIMENTS

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Where a range is expressed, a further aspect includes from the one particular value and/or to the other particular value. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g., ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

### GENERAL DEFINITIONS

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlett, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2^{nd} ed., J. Wiley & Sons (New York, N.Y. 1994), March, Advanced Organic Chemistry Reactions, Mechanisms and Structure 4^{th} ed., John Wiley & Sons (New York, N.Y. 1992); and Marten H. Hofker and Jan van Deursen, Transgenic Mouse Methods and Protocols, 2^{nd} edition (2011).

Definitions of common terms and techniques in chemistry and organic chemistry can be found in Smith. Organic Synthesis, published by Academic Press. 2016; Tinoco et al. Physical Chemistry, 5^{th} edition (2013) published by Pearson; Brown et al., Chemistry, The Central Science 14^{th} ed. (2017), published by Pearson, Clayden et al., Organic Chemistry, 2^{nd} ed. 2012, published by Oxford University Press; Carey and Sunberg, Advanced Organic Chemistry, Part A: Structure and Mechanisms, 5^{th} ed. 2008, published by Springer; Carey and Sunberg, Advanced Organic Chemistry, Part B: Reactions and Synthesis, 5^{th} ed. 2010, published by Springer, and Vollhardt and Schore, Organic Chemistry, Structure and Function; 8^{th} ed. (2018) published by W.H. Freeman.

Definitions of common terms, analysis, and techniques in genetics can be found in e.g., Hartl and Clark. Principles of Population Genetics. 4^{th} Ed. 2006, published by Oxford University Press. Published by Booker. Genetics: Analysis and Principles, 7^{th} Ed. 2021, published by McGraw Hill; Isik et la., Genetic Data Analysis for Plant and Animal Breeding. First ed. 2017. Published by Springer International Publishing AG; Green, E. L. Genetics and Probability in Animal Breeding Experiments. 2014, published by Palgrave; Bourdon, R. M. Understanding Animal Breeding. 2000 2^{nd} Ed. Published by Prentice Hall; Pal and Chakravarty. Genetics and Breeding for Disease Resistance of Livestock. First Ed. 2019, published by Academic Press; Fasso, D. Classification of Genetic Variance in Animals. First Ed. 2015, published by Callisto reference; Megahed, M. Handbook of Animal Breeding and Genetics, 2013, published by Omniscriptum Gmbh & Co. Kg., LAP Lambert Academic Publishing; Reece. Analysis of Genes and Genomes. 2004, published by John Wiley & Sons. Inc; Deonier et al., Computational Genome Analysis. 5^{th} Ed. 2005, published by Springer-Verlag, New York; Meneely, P. Genetic Analysis: Genes, Genomes, and Networks in Eukaryotes. 3^{rd} Ed. 2020, published by Oxford University Press.

As used herein, the singular forms “a”, “an”, and “the” include both singular and plural referents unless the context clearly dictates otherwise.

As used herein, “about,” “approximately,” “substantially,” and the like, when used in connection with a measurable variable such as a parameter, an amount, a temporal duration, and the like, are meant to encompass variations of and from the specified value including those within experimental error (which can be determined by e.g. given data set, art accepted standard, and/or with e.g., a given confidence interval (e.g., 90%, 95%, or more confidence interval from the mean), such as variations of +/−10% or less, +/−5% or less, +/−1% or less, and +/−0.1% or less of and from the specified value, insofar such variations are appropriate to perform in the disclosed invention. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

As used herein, a “biological sample” refers to a sample obtained from, made by, secreted by, excreted by, or otherwise containing part of or from a biologic entity. A biologic sample can contain whole cells and/or live cells and/or cell debris, and/or cell products, and/or virus particles. The biological sample can contain (or be derived from) a “bodily fluid”. The biological sample can be obtained from an environment (e.g., water source, soil, air, and the like). Such samples are also referred to herein as environmental samples. As used herein “bodily fluid” refers to any non-solid excretion, secretion, or other fluid present in an organism and includes, without limitation unless otherwise specified or is apparent from the description herein, amniotic fluid, aqueous humor, vitreous humor, bile, blood or component thereof (e.g., plasma, serum, etc.), breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from an organism, for example by puncture, or other collecting or sampling procedures.

The terms “subject,” “individual,” and “patient” are used interchangeably herein to refer to a vertebrate and non-vertebrate animals, including, but not limited to mammals and non-mammals. In some embodiments, the subject is a mammal. In some embodiments, the subject is a human, murine, simian, a farm animal (livestock, e.g., cows, pigs, chickens, sheep, goats, emu, bison, and the like), a sport animal (e.g., horses), a wild animal, or a pet animal (e.g., dog, cat, guinea pig, ferret, etc.). Non-mammal subjects include, but are not limited to, birds, fish, frogs, snakes, etc. In some embodiments, the subject is a non-human animal invertebrate including, but not limited to, a crustacean, e.g., a crab, lobster, or preferably a shrimp. Tissues, cells and their progeny of a biological entity obtained in vivo or cultured in vitro are also encompassed.

As used herein, “gene” refers to a hereditary unit corresponding to a sequence of DNA that occupies a specific location on a chromosome and that contains the genetic instruction for a characteristic(s) or trait(s) in an organism. The term gene can refer to translated and/or untranslated regions of a genome. “Gene” can refer to the specific sequence of DNA that is transcribed into an RNA transcript that can be translated into a polypeptide or be a catalytic RNA molecule, including but not limited to, tRNA, siRNA, piRNA, miRNA, long-non-coding RNA and shRNA. For the purpose of this disclosure, genes include regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions. In some embodiments, a gene can be transcribed to yield non-coding RNA, such that the RNA has a functional role to play in the organism.

As used herein, “identity” refers to a relationship between two or more nucleotide or polypeptide sequences, as determined by comparing the sequences. In the art, “identity” can also refer to the degree of sequence relatedness between nucleotide or polypeptide sequences as determined by the match between strings of such sequences. “Identity” can be readily calculated by known methods, including, but not limited to, those described in (Computational Molecular Biology, Lesk, A. M., Ed., Oxford University Press, New York, 1988; Biocomputing: Informatics and Genome Projects, Smith, D. W., Ed., Academic Press, New York, 1993; Computer Analysis of Sequence Data, Part I, Griffin, A. M, and Griffin, H. G., Eds., Humana Press, New Jersey, 1994; Sequence Analysis in Molecular Biology, von Heinje, G., Academic Press, 1987; and Sequence Analysis Primer, Gribskov, M and Devereux, J., Eds., M Stockton Press, New York, 1991; and Carillo, H., and Lipman, D., SIAM J. Applied Math. 1988, 48: 1073. Preferred methods to determine identity are designed to give the largest match between the sequences tested. Methods to determine identity are codified in publicly available computer programs. The percent identity between two sequences can be determined by using analysis software (e.g., Sequence Analysis Software Package of the Genetics Computer Group, Madison Wis.) that incorporates the Needelman and Wunsch, (J. Mol. Biol., 1970, 48: 443-453,) algorithm (e.g., NBLAST, and XBLAST). The default parameters are used to determine the identity for the polypeptides of the present disclosure, unless stated otherwise. Methods of determining identity can also be used to determine complementarity.

The term “molecular weight”, as used herein, generally refers to the mass or average mass of a material. If a polymer or oligomer, the molecular weight can refer to the relative average chain length or relative chain mass of the bulk polymer. In practice, the molecular weight of polymers and oligomers can be estimated or characterized in various ways including gel permeation chromatography (GPC) or capillary viscometry. GPC molecular weights are reported as the weight-average molecular weight (M_{w}) as opposed to the number-average molecular weight (Mₙ). Capillary viscometry provides estimates of molecular weight as the inherent viscosity determined from a dilute polymer solution using a particular set of concentration, temperature, and solvent conditions.

As used herein, “nucleic acid,” “nucleotide sequence,” and “polynucleotide” are used interchangeably herein and generally refer to a string of at least two base-sugar-phosphate combinations and refers to, among others, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, polynucleotide as used herein can refer to triple-stranded regions comprising RNA or DNA or both RNA and DNA. The strands in such regions can be from the same molecule or from different molecules. The regions may include all of one or more of the molecules, but more typically involve only a region of some of the molecules. One of the molecules of a triple-helical region often is an oligonucleotide. “Polynucleotide” and “nucleic acids” also encompasses such chemically, enzymatically or metabolically modified forms of polynucleotides, as well as the chemical forms of DNA and RNA characteristic of viruses and cells, including simple and complex cells, inter alia. For instance, the term polynucleotide as used herein can include DNAs or RNAs as described herein that contain one or more modified bases. Thus, DNAs or RNAs including unusual bases, such as inosine, or modified bases, such as tritylated bases, to name just two examples, are polynucleotides as the term is used herein. “Polynucleotide”, “nucleotide sequences” and “nucleic acids” also includes PNAs (peptide nucleic acids), phosphonothioates, and other variants of the phosphate backbone of native nucleic acids. Natural nucleic acids have a phosphate backbone, artificial nucleic acids can contain other types of backbones, but contain the same bases. Thus, DNAs or RNAs with backbones modified for stability or for other reasons are “nucleic acids” or “polynucleotides” as that term is intended herein. As used herein, “nucleic acid sequence” and “oligonucleotide” also encompasses a nucleic acid and polynucleotide as defined elsewhere herein.

As used herein, the term “specific binding” refers to covalent or non-covalent physical association of a first and a second moiety wherein the association between the first and second moieties is at least 2 times as strong, at least 5 times as strong as, at least 10 times as strong as, at least 50 times as strong as, at least 100 times as strong as, or stronger than the association of either moiety with most or all other moieties present in the environment in which binding occurs. Binding of two or more entities may be considered specific if the equilibrium dissociation constant, Kd, is 10⁻³ M or less, 10⁻⁴ M or less, 10⁻⁵ M or less, 10⁻⁶ M or less, 10⁻⁷ M or less, 10⁻⁸ M or less, 10⁻⁹ M or less, 10⁻¹⁰ M or less, 10⁻¹¹ M or less, or 10⁻¹² M or less under the conditions employed, e.g., under physiological conditions such as those inside a cell or consistent with cell survival. In some embodiments, specific binding can be accomplished by a plurality of weaker interactions (e.g., a plurality of individual interactions, wherein each individual interaction is characterized by a Kd of greater than 10⁻³ M). In some embodiments, specific binding, which can be referred to as “molecular recognition,” is a saturable binding interaction between two entities that is dependent on complementary orientation of functional groups on each entity. Examples of specific binding interactions include primer-polynucleotide interaction, aptamer-aptamer target interactions, antibody-antigen interactions, avidin-biotin interactions, ligand-receptor interactions, metal-chelate interactions, hybridization between complementary nucleic acids, etc.

As used herein, “tangible medium of expression” refers to a medium that is physically tangible or accessible and is not a mere abstract thought or an unrecorded spoken word. “Tangible medium of expression” includes, but is not limited to, words on a cellulosic or plastic material, or data stored in a suitable computer readable memory form. The data can be stored on a unit device, such as a flash memory or CD-ROM or on a server that can be accessed by a user via, e.g., a web interface.

As used herein, the terms “weight percent,” “wt %,” and “wt. %,” which can be used interchangeably, indicate the percent by weight of a given component based on the total weight of a composition of which it is a component, unless otherwise specified. That is, unless otherwise specified, all wt % values are based on the total weight of the composition. It should be understood that the sum of wt % values for all components in a disclosed composition or formulation are equal to 100. Alternatively, if the wt % value is based on the total weight of a subset of components in a composition, it should be understood that the sum of wt % values the specified components in the disclosed composition or formulation are equal to 100.

As used herein, “deoxyribonucleic acid (DNA)” and “ribonucleic acid (RNA)” can generally refer to any polyribonucleotide or polydeoxribonucleotide (collectively polynucleotides), which may be unmodified RNA or DNA or modified RNA or DNA. RNA can be in the form of non-coding RNA such as tRNA (transfer RNA), snRNA (small nuclear RNA), rRNA (ribosomal RNA), anti-sense RNA, RNAi (RNA interference construct), siRNA (short interfering RNA), microRNA (miRNA), or ribozymes, aptamers, guide RNA (gRNA) or coding mRNA (messenger RNA), long noncoding RNA (lncRNA) and/or the like.

As used herein, “fragment” as used throughout this specification with reference to a peptide, polypeptide, or protein generally denotes a portion of the peptide, polypeptide, or protein, such as typically an N- and/or C-terminally truncated form of the peptide, polypeptide, or protein. Preferably, a fragment may comprise at least about 30%, e.g., at least about 50% or at least about 70%, preferably at least about 80%, e.g., at least about 85%, more preferably at least about 90%, and yet more preferably at least about 95% or even about 99% of the amino acid sequence length of said peptide, polypeptide, or protein. For example, insofar not exceeding the length of the full-length peptide, polypeptide, or protein, a fragment may include a sequence of ≥5 consecutive amino acids, or ≥10 consecutive amino acids, or ≥20 consecutive amino acids, or ≥30 consecutive amino acids, e.g., ≥40 consecutive amino acids, such as for example ≥50 consecutive amino acids, e.g., ≥60, ≥70, ≥80, ≥90, ≥100, ≥200, ≥300, ≥400, ≥500 or ≥600 consecutive amino acids of the corresponding full-length peptide, polypeptide, or protein.

The term “fragment” with reference to a nucleic acid (polynucleotide) generally denotes a 5′- and/or 3′-truncated form of a nucleic acid. Preferably, a fragment may comprise at least about 30%, e.g., at least about 50% or at least about 70%, preferably at least about 80%, e.g., at least about 85%, more preferably at least about 90%, and yet more preferably at least about 95% or even about 99% of the nucleic acid sequence length of said nucleic acid. For example, insofar not exceeding the length of the full-length nucleic acid, a fragment may include a sequence of ≥5 consecutive nucleotides, or ≥10 consecutive nucleotides, or ≥20 consecutive nucleotides, or ≥30 consecutive nucleotides, e.g., ≥40 consecutive nucleotides, such as for example ≥50 consecutive nucleotides, e.g., ≥60, ≥70, ≥80, ≥90, ≥100, ≥200, ≥300, ≥400, ≥500 or ≥600 consecutive nucleotides of the corresponding full-length nucleic acid.

The terms encompass fragments arising by any mechanism, in vivo and/or in vitro, such as, without limitation, by alternative transcription or translation, exo- and/or endo-proteolysis, exo- and/or endo-nucleolysis, or degradation of the peptide, polypeptide, protein, or nucleic acid, such as, for example, by physical, chemical and/or enzymatic proteolysis or nucleolysis.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

As used herein “peptide” can refer to chains of at least 2 amino acids that are short, relative to a protein or polypeptide.

As used herein, “pharmaceutical formulation” refers to the combination of an active agent, compound, or ingredient with a pharmaceutically acceptable carrier or excipient, making the composition suitable for diagnostic, therapeutic, or preventive use in vitro, in vivo, or ex vivo.

As used herein, “pharmaceutically acceptable carrier or excipient” refers to a carrier or excipient that is useful in preparing a pharmaceutical formulation that is generally safe, non-toxic, and is neither biologically or otherwise undesirable, and includes a carrier or excipient that is acceptable for veterinary use as well as human pharmaceutical use. A “pharmaceutically acceptable carrier or excipient” as used in the specification and claims includes both one and more than one such carrier or excipient.

As used herein, “pharmaceutically acceptable salt” refers to any acid or base addition salt whose counter-ions are non-toxic to the subject to which they are administered in pharmaceutical doses of the salts.

As used herein, “plasmid” refers to a non-chromosomal double-stranded DNA sequence including an intact “replicon” such that the plasmid is replicated in a host cell.

As used herein, a “population” of cells is any number of cells greater than 1, but is preferably at least 1×10³ cells, at least 1×10⁴ cells, at least at least 1×10⁵ cells, at least 1×10⁶ cells, at least 1×10⁷ cells, at least 1×10⁸ cells, at least 1×10⁹ cells, or at least 1×10¹⁰ cells.

As used herein, “polypeptides” or “proteins” refers to amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V). “Protein” and “Polypeptide” can refer to a molecule composed of one or more chains of amino acids in a specific order. The term protein is used interchangeable with “polypeptide.” The order is determined by the base sequence of nucleotides in the gene coding for the protein. Proteins can be required for the structure, function, and regulation of the body's cells, tissues, and organs.

As used herein, “promoter” includes all sequences capable of driving transcription of a coding or a non-coding sequence. In particular, the term “promoter” as used herein refers to a DNA sequence generally described as the 5′ regulator region of a gene, located proximal to the start codon. The transcription of an adjacent coding sequence(s) is initiated at the promoter region. The term “promoter” also includes fragments of a promoter that are functional in initiating transcription of the gene.

As used herein, the term “recombinant” or “engineered” can generally refer to a non-naturally occurring nucleic acid, nucleic acid construct, or polypeptide. Such non-naturally occurring nucleic acids may include natural nucleic acids that have been modified, for example that have deletions, substitutions, inversions, insertions, etc., and/or combinations of nucleic acid sequences of different origin that are joined using molecular biology technologies (e.g., a nucleic acid sequences encoding a fusion protein (e.g., a protein or polypeptide formed from the combination of two different proteins or protein fragments), the combination of a nucleic acid encoding a polypeptide to a promoter sequence, where the coding sequence and promoter sequence are from different sources or otherwise do not typically occur together naturally (e.g., a nucleic acid and a constitutive promoter), etc. Recombinant or engineered can also refer to the polypeptide encoded by the recombinant nucleic acid. Non-naturally occurring nucleic acids or polypeptides include nucleic acids and polypeptides modified by man.

As used herein, “targeting moiety” refers to molecules, complexes, agents, and the like that is capable of specifically or selectively interacting with, binding with, acting on or with, or otherwise associating or recognizing a target molecule, agent, and/or complex that is associated with, part of, coupled to, another object, complex, surface, and the like, such as a cell or cell population, tissue, organ, subcellular locale, object surface, particle etc. Targeting moieties can be chemical, biological, metals, polymers, or other agents and molecules with targeting capabilities. Targeting moieties can be amino acids, peptides, polypeptides, nucleic acids, polynucleotides, lipids, sugars, metals, small molecule chemicals, combinations thereof, and the like. Targeting moieties can be antibodies or fragments thereof, aptamers, DNA, RNA such as guide RNA for a RNA guided nuclease or system, ligands, substrates, enzymes, combinations thereof, and the like. The specificity or selectivity of a targeting moiety can be determined by any suitable method or technique that will be appreciated by those of ordinary skill in the art. For example, in some embodiments, the methods described herein include determining the disassociation constant for the targeting moiety and target. In some embodiments, the targeting moiety has a specificity the equilibrium dissociation constant, Kd, is 10⁻³ M or less, 10⁻⁴ M or less, 10⁻⁵ M or less, 10⁻⁶ M or less, 10⁻⁷ M or less, 10⁻⁸ M or less, 10⁻⁹ M or less, 10⁻¹⁰ M or less, 10⁻¹¹ M or less, or 10⁻¹² M or less under the conditions employed, e.g., under physiological conditions such as those inside a cell or consistent with cell survival. In some embodiments, specific binding can be accomplished by a plurality of weaker interactions (e.g., a plurality of individual interactions, wherein each individual interaction is characterized by a Kd of greater than 10⁻³ M). In some embodiments, the targeting moiety has increased binding with, association with, interaction with, activity on as compared to non-targets, such as a 1 to 500 or more fold increase. Targets of targeting moieties can be amino acids, peptides, polypeptides, nucleic acids, polynucleotides, lipids, sugars, metals, small molecule chemicals, combinations thereof, and the like. Targets can be receptors, biomarkers, transporters, antigens, complexes, combinations thereof, and the like.

All publications, published patent documents, and patent applications cited herein are hereby incorporated by reference to the same extent as though each individual publication, published patent document, or patent application was specifically and individually indicated as being incorporated by reference.

### Overview

Promoters are the primary driver for gene expression in a homologous or heterologous expression system. Availability of a promoter element capable of driving a gene expression in a constitutive or inducive manner is fundamental in understanding the biological function of a protein molecule in a cell as well as leveraging expression for various practical applications. For example, a strong promoter is often needed for driving expression of therapeutic protein in a heterologous expression system, such as in bacteria, yeast, fungi, insect cells, mammalian cells and in plant cells.

With that said, embodiments disclosed herein can provide nudiviral promoters that can be useful to drive expression of polynucleotides in various contexts. Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

### Nudiviral Nucleic Acids, Engineered Polynucleotides, and Vectors

### Nudiviral Nucleic Acids

Described in certain example embodiments herein are nudiviral nucleic acids corresponding to a polyhedrin gene promoter of a nudivirus, a variant thereof, or a derivative thereof. In certain example embodiments, the nudivirus is an alpha nudivirus, betanudivirus, deltanudavirus, gammanudavirus, or an epsilonnudavirus. In certain example embodiments, the nudivirus is a shrimp, fish, or insect nudivirus. In certain example embodiments, the nucleic acid comprises or consists of a sequence corresponding to SEQ ID NO: 5, 6, 7, or 8. In certain example embodiments, the nucleic acid comprises or consists of a sequence corresponding to SEQ ID NO: 7. In certain example embodiments, the nucleic acid comprises or consists of a sequence that is 80-100% identical to SEQ ID Nos: 5, 6, 7, or 8, optionally SEQ ID NO: 7. In certain example embodiments, the nucleic acid comprises or consists of a sequence that is 80-100% identical to SEQ ID NO: 7.

In certain example embodiments, the nucleic acid comprises or consists of a sequence that is 80%, to/or 81%, 82%, 83%, 84%, 85%, 86%, 87%, 88%, 89%, 90%, 91%, 92%, 93%, 94%, 95%, 96%, 97%, 98%, 99%, 100% identical to SEQ ID Nos: SEQ ID NO: 5, 6, 7, or 8. In some embodiments, the nucleic acid comprises or consists of a sequence that is that is 80%, to/or 81%, 82%, 83%, 84%, 85%, 86%, 87%, 88%, 89%, 90%, 91%, 92%, 93%, 94%, 95%, 96%, 97%, 98%, 99%, 100% identical to SEQ ID NO: 7.

### Engineered Polynucleotides

Described in certain example embodiments herein are engineered polynucleotides comprising the nudiviral nucleic acid of the present description; and a polynucleotide encoding a non-polyhedrin polynucleotide and/or polypeptide, wherein the nudiviral nucleic acid is operatively coupled to the polynucleotide encoding a non-polyhedrin polynucleotide.

It will be appreciated that the nudiviral nucleic acid can be used to drive the expression of a broad range of nucleic acids, such as those that encode RNAs and/or proteins of interest. As used herein, “expression” refers to the process by which polynucleotides are transcribed into RNA transcripts. In the context of mRNA and other translated RNA species, “expression” also refers to the process or processes by which the transcribed RNA is subsequently translated into peptides, polypeptides, or proteins. In some instances, “expression” is a reflection of the stability of a given RNA. For example, when one measures RNA, depending on the method of detection and/or quantification of the RNA as well as other techniques used in conjunction with RNA detection and/or quantification, it can be that increased/decreased RNA transcript levels are the result of increased/decreased transcription and/or increased/decreased stability and/or degradation of the RNA transcript. One of ordinary skill in the art will appreciate these techniques and the relation “expression” in these various contexts to the underlying biological mechanisms.

In some embodiments, the non-polyhedrin polynucleotide is a gene of interest. In some embodiments, the non-polyhedrin polynucleotide encodes an RNA and/or protein.

### Vectors and Vector Systems

Described in certain example embodiments herein are vectors or vector systems comprising a nudiviral nucleic acid of the present description, an engineered polynucleotide of the present description, or both. In some embodiments, In certain example embodiments, the vector is an expression vector or vector system. Vector systems include those that involve two or more vectors that can be used together, such as for expression, viral packaging, or other control of nucleotide expression or delivery.

In some embodiments the nudiviral nucleic acid and/or engineered polynucleotide of the present description is incorporated into a vector or vector system or particle, such as a virus or viral like particle, produced from such a vector or vector system. In certain embodiments, the vector can contain one or more polynucleotides encoding one or more elements (for example, genes) to be transcribed from the nudiviral nucleic acids. Exemplary elements are described elsewhere herein, such as associated with an engineered polynucleotide of the present description. The vectors can be useful in producing bacterial, fungal, yeast, insects plants, human and non-human animal cells, and non-human organisms and that can express an element (e.g., a gene) from the nudiviral nucleic acids of the present description.

The vectors and/or vector systems can be used, for example, to express one or more of the polynucleotides in a cell, such as a producer cell, to produce virus or virus-like particles containing an element that is transcribed from a nudiviral nucleic acid of the present description described elsewhere herein. Other uses for the vectors and vector systems described herein are also within the scope of this disclosure. In general, and throughout this specification, the term “vector” refers to a tool that allows or facilitates the transfer of an entity from one environment to another. In some contexts which will be appreciated by those of ordinary skill in the art, “vector” can be a term of art to refer to a nucleic acid molecule capable of transporting another nucleic acid to which it has been linked. A vector can be a replicon, such as a plasmid, phage, or cosmid, into which another DNA segment may be inserted so as to bring about the replication of the inserted segment. Generally, a vector is capable of replication when associated with the proper control elements.

Vectors include, but are not limited to, nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g., retroviruses, replication defective retroviruses, adenoviruses, replication defective adenoviruses, and adeno-associated viruses (AAVs)). Viral vectors also include polynucleotides carried by a virus for transfection into a host cell. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” Common expression vectors of utility in recombinant DNA techniques are often in the form of plasmids.

Recombinant expression vectors can be composed of a nucleic acid (e.g., a polynucleotide) of the invention in a form suitable for expression of the nucleic acid in a host cell, which means that the recombinant expression vectors include one or more regulatory elements, which can be selected on the basis of the host cells to be used for expression, that is operatively-linked to the nucleic acid sequence to be expressed. Within a recombinant expression vector, “operably linked” and “operatively-linked” are used interchangeably herein and mean that a nucleotide sequence of interest (such as that of a gene) is linked to the regulatory element(s) (including, but not limited to, a nudiviral nucleic acid of the present disclosure) in a manner that allows for expression of the nucleotide sequence (e.g., in an in vitro transcription/translation system or in a host cell when the vector is introduced into the host cell). Advantageous vectors include lentiviruses and adeno-associated viruses, and types of such vectors can also be selected for targeting particular types of cells. These and other embodiments of the vectors and vector systems are described elsewhere herein. In some embodiments, the vector is suitable for expression in a shrimp. In some embodiments, the vector is a nodaviral vector, such as that set for in International Application No.: PCT/US2022/035858. In some embodiments, the vector can be a bicistronic vector.

### Cell-based Vector Amplification and Expression

Vectors may be introduced and propagated in a prokaryotic cell or eukaryotic cell. In some embodiments, a prokaryote is used to amplify copies of a vector to be introduced into a eukaryotic cell or as an intermediate vector in the production of a vector to be introduced into a eukaryotic cell (e.g., amplifying a plasmid as part of a viral vector packaging system). The vectors can be viral-based or non-viral based. In some embodiments, a prokaryote is used to amplify copies of a vector and express one or more nucleic acids, such as to provide a source of one or more proteins for delivery to a host cell or host organism.

Vectors can be designed for expression of one or more elements (e.g., genes or nucleic acids) in a suitable host cell. In some embodiments, the suitable host cell is a prokaryotic cell. Suitable host cells include, but are not limited to, bacterial cells, yeast cells, insect cells, and mammalian cells. In some embodiments, the suitable host cell is a eukaryotic cell. In some embodiments the host cell is a producer cell capable of producing particles (e.g., virus particles, virus like particles, exosomes, and/or the like) that can be used to deliver an engineered polynucleotide of the present description to a cell.

In some embodiments, the suitable host cell is a suitable bacterial cell. Suitable bacterial cells include but are not limited to bacterial cells from the bacteria of the species Escherichia coli. Many suitable strains of E. coli are known in the art for expression of vectors. These include, but are not limited to Pir1, Stb12, Stb13, Stb14, TOP10, XL1 Blue, and XL10 Gold. In some embodiments, the host cell is a suitable insect cell. Suitable insect cells include those from Spodoptera frugiperda. Suitable strains of S. frugiperda cells include, but are not limited to, Sf9 and Sf21. In some embodiments, the host cell is a suitable yeast cell. In some embodiments, the yeast cell can be from Saccharomyces cerevisiae. In some embodiments, the host cell is a suitable mammalian cell. Many types of mammalian cells have been developed to express vectors. Suitable mammalian cells include, but are not limited to, HEK293, Chinese Hamster Ovary Cells (CHOs), mouse myeloma cells, HeLa, U2OS, A549, HT1080, CAD, P19, NIH 3T3, L929, N2a, MCF-7, Y79, SO-Rb50, HepG G2, DIKX-X11, J558L, Baby hamster kidney cells (BHK), and chicken embryo fibroblasts (CEFs). Suitable host cells are discussed further in Goeddel, GENE EXPRESSION TECHNOLOGY: METHODS IN ENZYMOLOGY 185, Academic Press, San Diego, Calif (1990). In some embodiments, the suitable host cell is a bovine cell, including but not limited to, bovine embryonic stem cells, bovine induced pluripotent stem cells, bovine blastocyst cells, bovine spermatogonia stem cells, bovine oogonial cells, bovine primordial germ cells, bovine primordial germ cell like cells, bovine totipotent cells, or other bovine cell described elsewhere herein.

In some embodiments, the vector is a yeast expression vector. Examples of vectors for expression in yeast Saccharomyces cerevisiae include pYepSec1 (Baldari, et al., 1987. EMBO J. 6: 229-234), pMFa (Kuijan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif). As used herein, a “yeast expression vector” refers to a nucleic acid that contains one or more sequences encoding an RNA and/or polypeptide and may further contain any desired elements that control the expression of the nucleic acid(s), as well as any elements that enable the replication and maintenance of the expression vector inside the yeast cell. Many suitable yeast expression vectors and features thereof are known in the art; for example, various vectors and techniques are illustrated in in Yeast Protocols, 2^{nd} edition, Xiao, W., ed. (Humana Press, New York, 2007) and Buckholz, R. G. and Gleeson, M. A. (1991) Biotechnology (NY) 9(11): 1067-72. Yeast vectors can contain, without limitation, a centromeric (CEN) sequence, an autonomous replication sequence (ARS), a promoter, such as a nudiviral nucleic acid of the present description and, operably linked to a sequence or gene of interest, a terminator such as an RNA polymerase III terminator, an origin of replication, and a marker gene (e.g., auxotrophic, antibiotic, or other selectable markers). Examples of expression vectors for use in yeast may include plasmids, yeast artificial chromosomes, 2μ plasmids, yeast integrative plasmids, yeast replicative plasmids, shuttle vectors, and episomal plasmids. It will be appreciated that the vector can contain more than one nucleic acid to be expressed and that other promoters, such as a RNA polymerase II or III promoter can be used to drive nucleic acid expression in addition to a nudiviral nucleic acid of the present description.

In some embodiments, the vector is a baculovirus vector or expression vector and can be suitable for expression of polynucleotides and/or proteins in insect cells. In some embodiments, the suitable host cell is an insect cell. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39). rAAV (recombinant Adeno-associated viral) vectors are preferably produced in insect cells, e.g., Spodoptera frugiperda Sf9 insect cells, grown in serum-free suspension culture. Serum-free insect cells can be purchased from commercial vendors, e.g., Sigma Aldrich (EX-CELL 405).

In some embodiments, the vector is a mammalian expression vector. In some embodiments, the mammalian expression vector is capable of expressing one or more polynucleotides and/or polypeptides in a mammalian cell. Examples of mammalian expression vectors include, but are not limited to, pCDM8 (Seed, 1987. Nature 329: 840) and pMT2PC (Kaufman, et al., 1987. EMBO J. 6: 187-195). The mammalian expression vector can include one or more suitable regulatory elements capable of controlling expression of the one or more polynucleotides and/or proteins in the mammalian cell. For example, commonly used promoters are derived from polyoma, adenovirus 2, cytomegalovirus, simian virus 40, and others disclosed herein and known in the art. More detail on suitable regulatory elements is provided elsewhere herein.

For other suitable expression vectors and vector systems in which the nudiviral nucleic acid and/or engineered polynucleotide of the present disclosure can be incorporated for both prokaryotic and eukaryotic cells see, e.g., Chapters 16 and 17 of Sambrook, et al., MOLECULAR CLONING: A LABORATORY MANUAL. 2^{nd} ed., Cold Spring Harbor Laboratory, Cold Spring Harbor Laboratory Press, Cold Spring Harbor, N.Y., 1989.

In some embodiments, the vector can be a fusion vector or fusion expression vector. In some embodiments, fusion vectors add a number of amino acids to a protein encoded therein, such as to the amino terminus, carboxy terminus, or both of a recombinant protein. Such fusion vectors can serve one or more purposes, such as: (i) to increase expression of recombinant protein; (ii) to increase the solubility of the recombinant protein; and (iii) to aid in the purification of the recombinant protein by acting as a ligand in affinity purification. In some embodiments, expression of polynucleotides (such as non-coding polynucleotides) and proteins in prokaryotes can be carried out in Escherichia coli with vectors containing constitutive or inducible promoters directing the expression of either fusion or non-fusion polynucleotides and/or proteins. In some embodiments, the fusion expression vector can include a proteolytic cleavage site, which can be introduced at the junction of the fusion vector backbone or other fusion moiety and the recombinant polynucleotide or protein to enable separation of the recombinant polynucleotide or protein from the fusion vector backbone or other fusion moiety subsequent to purification of the fusion polynucleotide or protein. Such enzymes, and their cognate recognition sequences, include Factor Xa, thrombin and enterokinase. Example fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein. Examples of suitable inducible non-fusion E. coli expression vectors include pTrc (Amrann et al., (1988) Gene 69:301-315) and pET lid (Studier et al., GENE EXPRESSION TECHNOLOGY: METHODS IN ENZYMOLOGY 185, Academic Press, San Diego, Calif. (1990) 60-89).

### Cell-Free Vector and Polynucleotide Expression

In some embodiments, the nudiviral nucleic acid of the present disclosure is used to express a polynucleotide of interest (e.g., a gene) from a vector or suitable polynucleotide in a cell-free in vitro system. In other words, an engineered polynucleotide of the present description can be transcribed and optionally translated in vitro. In vitro transcription/translation systems and appropriate vectors are generally known in the art and commercially available. Generally, in vitro transcription and in vitro translation systems replicate the processes of RNA and protein synthesis, respectively, outside of the cellular environment. Vectors and suitable polynucleotides for in vitro transcription can include T7, SP6, T3, promoter regulatory sequences that can be recognized and acted upon by an appropriate polymerase to transcribe the polynucleotide or vector.

In vitro translation can be stand-alone (e.g., translation of a purified polyribonucleotide) or linked/coupled to transcription. In some embodiments, the cell-free (or in vitro) translation system can include extracts from rabbit reticulocytes, wheat germ, and/or E. coli. The extracts can include various macromolecular components that are needed for translation of exogenous RNA (e.g., 70S or 80S ribosomes, tRNAs, aminoacyl-tRNA, synthetases, initiation, elongation factors, termination factors, etc.). Other components can be included or added during the translation reaction, including but not limited to, amino acids, energy sources (ATP, GTP), energy regenerating systems (creatine phosphate and creatine phosphokinase (eukaryotic systems)) (phosphoenol pyruvate and pyruvate kinase for bacterial systems), and other co-factors (Mg²⁺, K⁺, etc.). As previously mentioned, in vitro translation can be based on RNA or DNA starting material. Some translation systems can utilize an RNA template as starting material (e.g., reticulocyte lysates and wheat germ extracts). Some translation systems can utilize a DNA template as a starting material (e.g., E coli-based systems). In these systems transcription and translation are coupled and DNA is first transcribed into RNA, which is subsequently translated. Suitable standard and coupled cell-free translation systems are generally known in the art and are commercially available.

### Vector Features

The vectors can include additional features that can confer one or more functionalities to the vector, the polynucleotide to be delivered and/or expressed, a virus or other particle (e.g., viral like particle or exosome) produced there from, or polypeptide expressed therefrom. Such features include, but are not limited to, regulatory elements (e.g., promoters, enhancers, repressors, etc.), selectable markers, molecular identifiers (e.g., molecular barcodes), stabilizing elements, and the like. It will be appreciated by those skilled in the art that the design of the expression vector and additional features included can depend on such factors as the choice of the host cell to be transformed, the level of expression desired, etc.

### Regulatory Elements

In certain embodiments, the polynucleotides and/or vectors thereof described herein (such as the genetic modifying system polynucleotides described herein) can include one or more regulatory elements that can be operatively linked to the polynucleotide. The term “regulatory element” is intended to include promoters, enhancers, internal ribosomal entry sites (IRES), other expression control elements (e.g., transcription termination signals, such as polyadenylation signals and poly-U sequences) and cellular localization signals (e.g., nuclear localization or export signals). In some embodiments, a promoter included in the vector is a nudiviral nucleic acid of the present disclosure. In some embodiments, a promoter included in the vector is identical to any one of SEQ ID Nos: 5-8, optionally SEQ ID NO: 7. In some embodiments, promoter included in the vector is 80-100% identical to any one of SEQ ID Nos: 5-8, optionally SEQ ID NO: 7. Additional regulatory elements are described, for example, in Goeddel, GENE EXPRESSION TECHNOLOGY: METHODS IN ENZYMOLOGY 185, Academic Press, San Diego, Calif (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter can direct expression primarily in a desired tissue of interest, such as muscle, neuron, bone, skin, blood, specific organs (e.g., liver, pancreas), or particular cell types (e.g., lymphocytes). Regulatory elements may also direct expression in a temporal-dependent manner, such as in a cell-cycle dependent or developmental stage-dependent manner, which may or may not also be tissue or cell-type specific. In some embodiments, a vector comprises one or more pol III promoter (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) (see, e.g., Boshart et al, Cell, 41:521-530 (1985)), the SV40 promoter, the dihydrofolate reductase promoter, the β-actin promoter, the phosphoglycerol kinase (PGK) promoter, and the EF1α promoter. Also encompassed by the term “regulatory element” are enhancer elements, such as WPRE; CMV enhancers; the R-U5′ segment in LTR of HTLV-I (Mol. Cell. Biol., Vol. 8(1), p. 466-472, 1988); SV40 enhancer; and the intron sequence between exons 2 and 3 of rabbit β-globin (Proc. Natl. Acad. Sci. USA., Vol. 78(3), p. 1527-31, 1981). Exemplary promoters also include bovine U6 (bU6) and bovine 7SK (b7SK), and other bovine PolII promoters (see e.g., Lambeth et al., Anim Genet. 2006 August; 37(4):369-72), bovine papillomavirus-1 promoters (BPV-1) (Linz and Baker. J Virol. 1988 August; 62(8):2537-43. Doi: 10.1128/JVI.62.8.2537-2543.1988), the bovine SIX1 gene promoter (see e.g., Wei et al. Scientific Reports volume 7, Article number: 12599 (2017)), bovine growth hormone promoter (see e.g., Jiang et al., Nuc Acid Prot Syn Mol Gen. 1999. 274(12): 7893-7900), bovine pyruvate carboxylase (see e.g., Hazelton et al. J. Dairy Sci. 91:91-99), a bidirectional promoter (see e.g., Meersserman et al. DNA Research, Volume 24, Issue 3, June 2017, Pages 221-233), a bovine Akt3 promoter (see e.g., Farmanullah et al. Journal of Genetic Engineering and Biotechnology (2021) 19:164), bovine alpha-lactalbumin promoter (see e.g., FEBS Lett. 1991 Jun. 17; 284(1):19-22), bovine beta-casein promoter (see e.g., Cerdan et al., Mol Reprod Dev. 1998 March; 49(3):236-45), any combination thereof.

In some embodiments, the vector includes a plant promoter. The term “plant promoter” as used herein is a promoter capable of initiating transcription in plant cells, whether or not its origin is a plant cell. Exemplary suitable plant promoters include, but are not limited to, those that are obtained from plants, plant viruses, and bacteria such as Agrobacterium or Rhizobium which comprise genes expressed in plant cells.

The use of different types of promoters is envisaged. A constitutive plant promoter is a promoter that is able to express the open reading frame (ORF) that it controls in all or nearly all of the plant tissues during all or nearly all developmental stages of the plant (referred to as “constitutive expression”). One non-limiting example of a constitutive promoter is the cauliflower mosaic virus 35S promoter. Different promoters may direct the expression of a gene in different tissues or cell types, or at different stages of development, or in response to different environmental conditions. Additional plant promoters that can be included in a vector of the present disclosure are found in Kawamata et al., (1997) Plant Cell Physiol 38:792-803; Yamamoto et al., (1997) Plant J 12:255-65; Hire et al, (1992) Plant Mol Biol 20:207-18, Kuster et al, (1995) Plant Mol Biol 29:759-72, and Capana et al., (1994) Plant Mol Biol 25:681-91.

In some embodiments, the regulatory sequence is a regulatory sequence described in U.S. Pat. No. 7,776,321, U.S. Pat. Pub. No. 2011/0027239, or International Patent Publication No. WO 2011/028929, the contents of which are incorporated by reference herein in their entireties. In some embodiments, the vector can contain a minimal promoter. In some embodiments, the minimal promoter is the Mecp2 promoter, tRNA promoter, or U6. In a further embodiment, the minimal promoter is tissue specific. In some embodiments, the length of the vector polynucleotide the minimal promoters and polynucleotide sequences is less than 4.4 Kb.

To express a polynucleotide, the vector can include one or more transcriptional and/or translational initiation regulatory sequences, e.g., promoters, that direct the transcription of the gene and/or translation of the encoded protein in a cell. In some embodiments a constitutive promoter may be employed. In some embodiments, a promoter included in the vector is a nudiviral nucleic acid of the present disclosure. In some embodiments, a promoter included in the vector is identical to any one of SEQ ID Nos: 5-8, optionally SEQ ID NO: 7. In some embodiments, promoter included in the vector is 80-100% identical to any one of SEQ ID Nos: 5-8, optionally SEQ ID NO: 7. Other suitable constitutive promoters for mammalian cells are generally known in the art and include, but are not limited to SV40, CAG, CMV, EF-1α, β-actin, RSV, and PGK. Suitable constitutive promoters for bacterial cells, yeast cells, and fungal cells are generally known in the art, such as a T-7 promoter for bacterial expression and an alcohol dehydrogenase promoter for expression in yeast.

In some embodiments, the regulatory element can be a regulated promoter. As used herein, “regulated promoter” refers to promoters that direct gene expression not constitutively, but in a temporally- and/or spatially-regulated manner, and includes tissue-specific, tissue-preferred and inducible promoters. Regulated promoters include conditional promoters and inducible promoters. In some embodiments, conditional promoters can be employed to direct expression of a polynucleotide in a specific cell type, under certain environmental conditions, and/or during a specific state of development. Suitable tissue specific promoters can include, but are not limited to, liver specific promoters (e.g., APOA2, SERPIN A1 (hAAT), CYP3A4, and MIR122), pancreatic cell promoters (e.g., INS, IRS2, Pdx1, Alx3, Ppy), cardiac specific promoters (e.g., Myh6 (alpha MHC), MYL2 (MLC-2v), TNI3 (cTn1), NPPA (ANF), Slc8al (Ncxl)), central nervous system cell promoters (SYN1, GFAP, INA, NES, MOBP, MBP, TH, FOXA2 (HNF3 beta)), skin cell specific promoters (e.g., FLG, K14, TGM3), immune cell specific promoters, (e.g., ITGAM, CD43 promoter, CD14 promoter, CD45 promoter, CD68 promoter), urogenital cell specific promoters (e.g., Pbsn, Upk2, Sbp, Ferl14), endothelial cell specific promoters (e.g., ENG), pluripotent and embryonic germ layer cell specific promoters (e.g., Oct4, NANOG, Synthetic Oct4, T brachyury, NES, SOX17, FOXA2, MIR122), and muscle cell specific promoter (e.g., myostatin, Desmin). Other tissue and/or cell specific promoters are generally known in the art and are within the scope of this disclosure.

Inducible/conditional promoters can be positively inducible/conditional promoters (e.g., a promoter that activates transcription of the polynucleotide upon appropriate interaction with an activated activator, or an inducer (compound, environmental condition, or other stimulus) or a negative/conditional inducible promoter (e.g., a promoter that is repressed (e.g., bound by a repressor) until the repressor condition of the promotor is removed (e.g., inducer binds a repressor bound to the promoter stimulating release of the promoter by the repressor or removal of a chemical repressor from the promoter environment). The inducer can be a compound, environmental condition, or other stimulus. Thus, inducible/conditional promoters can be responsive to any suitable stimuli such as chemical, biological, or other molecular agents, temperature, light, and/or pH. Suitable inducible/conditional promoters include, but are not limited to, Tet-On, Tet-Off, Lac promoter, pBad, AlcA, LexA, Hsp70 promoter, Hsp90 promoter, pDawn, XVE/OlexA, GVG, and pOp/LhGR.

Examples of promoters that are inducible and that can allow for spatiotemporal control of gene editing or gene expression may use a form of energy. The form of energy may include but is not limited to sound energy, electromagnetic radiation, chemical energy and/or thermal energy. Examples of inducible systems include tetracycline inducible promoters (Tet-On or Tet-Off), small molecule two-hybrid transcription activations systems (FKBP, ABA, etc.), or light inducible systems (Phytochrome, LOV domains, or cryptochrome), such as a Light Inducible Transcriptional Effector (LITE) that direct changes in transcriptional activity in a sequence-specific manner. The components of a light inducible system may include one or more elements of interest (e.g., genes), a light-responsive cytochrome heterodimer (e.g., from Arabidopsis thaliana), and a transcriptional activation/repression domain. In some embodiments, the vector can include one or more of the inducible DNA binding proteins provided in International Patent Publication No. WO 2014/018423 and U.S. Patent Publication Nos., 2015/0291966, 2017/0166903, 2019/0203212, which describe e.g., embodiments of inducible DNA binding proteins and methods of use and can be adapted for use with the present invention.

In some embodiments, transient or inducible expression can be achieved by including, for example, chemical-regulated promotors, i.e., whereby the application of an exogenous chemical induces gene expression. Modulation of gene expression can also be obtained by including a chemical-repressible promoter, where application of the chemical represses gene expression. Chemical-inducible promoters include, but are not limited to, the maize ln2-2 promoter, activated by benzene sulfonamide herbicide safeners (De Veylder et al., (1997) Plant Cell Physiol 38:568-77), the maize GST promoter (GST-ll-27, WO93/01294), activated by hydrophobic electrophilic compounds used as pre-emergent herbicides, and the tobacco PR-1 a promoter (Ono et al., (2004) Biosci Biotechnol Biochem 68:803-7) activated by salicylic acid. Promoters that are regulated by antibiotics, such as tetracycline-inducible and tetracycline-repressible promoters (Gatz et al., (1991) Mol Gen Genet 227:229-37; U.S. Pat. Nos. 5,814,618 and 5,789,156) can also be used herein.

In some embodiments where multiple elements are to be expressed from the same vector or within the same vector system, different promoters or regulatory elements can be used for each element to be expressed to avoid or limit loss of expression due to competition between promoters and/or other regulatory elements. In some embodiments, at least one of the promoters is a nudiviral nucleic acid of the present disclosure. In some embodiments, at least one of the promoters included in the vector is identical to any one of SEQ ID NO: 5-8. In some embodiments, at least one of the promoters included in the vector is identical to SEQ ID NO: 7. In some embodiments, at least one of the promoters included in the vector is 80%-100% identical to any one of SEQ ID NO: 5-8. In some embodiments, at least one of the promoters included in the vector is 80%-100% identical to SEQ ID NO: 7. In some embodiments, at least one of the promoters included in the vector is 80%, to/or 81%, 82%, 83%, 84%, 85%, 86%, 87%, 88%, 89%, 90%, 91%, 92%, 93%, 94%, 95%, 96%, 97%, 98%, 99%, 100% identical to any one of SEQ ID NO: 5-8. In some embodiments, at least one of the promoters included in the vector is 80%, to/or 81%, 82%, 83%, 84%, 85%, 86%, 87%, 88%, 89%, 90%, 91%, 92%, 93%, 94%, 95%, 96%, 97%, 98%, 99%, 100% identical to SEQ ID NO: 7.

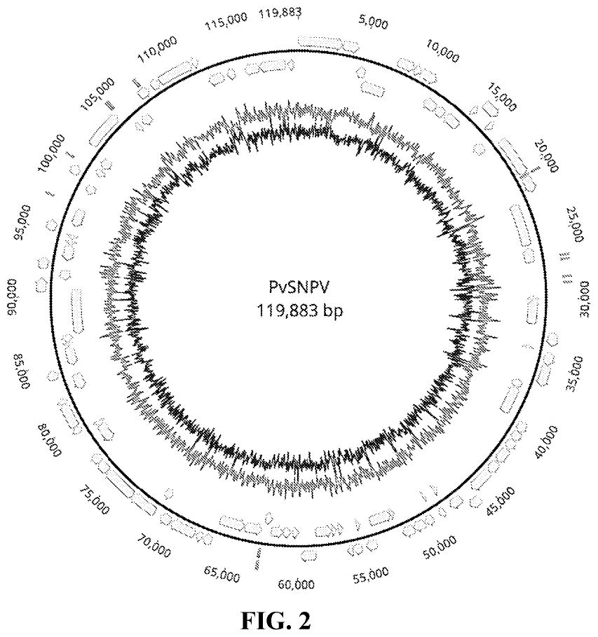

In some embodiments, the polynucleotide, vector or system thereof can include one or more elements capable of translocating and/or expressing a polynucleotide to/in a specific cell component or organelle. Such organelles can include, but are not limited to, nucleus, ribosome, endoplasmic reticulum, Golgi apparatus, chloroplast, mitochondria, vacuole, lysosome, cytoskeleton, plasma membrane, cell wall, peroxisome, centrioles, etc. Such regulatory elements can include, but are not limited to, nuclear localization signals (examples of which are described in greater detail elsewhere herein), any such as those that are annotated in the LocSigDB database (see e.g., genome.unmc.edu/LocSigDB/and Negi et al., 2015. Database. 2015: bav003; doi: 10.1093/database/bav003), nuclear export signals (e.g., LXXXLXXLXL (SEQ ID NO: 17) and others described elsewhere herein), endoplasmic reticulum localization/retention signals (e.g., KDEL (SEQ ID NO: 18), KDXX, KKXX, KXX, and others described elsewhere herein; and see e.g., Liu et al. 2007 Mol. Biol. Cell. 18(3):1073-1082 and Gorleku et al., 2011. J. Biol. Chem. 286:39573-39584), mitochondria targeting signals (see e.g., Chin, R. M., et al, 2018, Cell Reports. 22:2818-2826, particularly at FIG. 2; Doyle et al. 2013. PLoS ONE 8, e67938; Funes et al. 2002. J. Biol. Chem. 277:6051-6058; Matouschek et al. 1997. PNAS USA 85:2091-2095; Oca-Cossio et al., 2003. 165:707-720; Waltner et al., 1996. J. Biol. Chem. 271:21226-21230; Wilcox et al., 2005. PNAS USA 102:15435-15440; Galanis et al., 1991. FEBS Lett 282:425-430), and peroxisome targeting signals (e.g. (S/A/C)-(K/R/H)-(L/A), SLK, (R/K)-(L/V/I)-XXXXX-(H/Q)-(L/A/F)). Suitable protein targeting motifs can also be designed or identified using any suitable database or prediction tool, including but not limited to Minimotif Miner (minimotifminer.org, mitominer.mrc-mbu.cam.ac.uk/release-4.0/embodiment.do?name=Protein %20MTS), LocDB (see above), PTSs predictor, TargetP-2.0 www.cbs.dtu.dk/services/TargetP/), ChloroP (www.cbs.dtu.dk/services/ChloroP/); NetNES (www.cbs.dtu.dk/services/NetNES/), Predotar (urgi.versailles.inra.fr/predotar/), and SignalP (www.cbs.dtu.dk/services/SignalP/).

### Selectable Markers and Tags

One or more of the polynucleotides and/or vectors described herein, such as those of or encoding a genetic modifying system and/or exogenous gene can be operably linked, fused to, or otherwise modified to include a polynucleotide that encodes or is a selectable marker or tag, which can be a polynucleotide or polypeptide. In some embodiments, the polypeptide encoding a polypeptide selectable marker is incorporated in the genetic modifying system polynucleotide or other polynucleotide of the present disclosure such that the selectable marker polypeptide, when translated, is inserted between two amino acids between the N- and C-terminus of the genetic modifying system polypeptide (or other polypeptide of the present disclosure) or at the N- and/or C-terminus of the genetic modifying system polypeptide (or other polypeptide of the present disclosure). In some embodiments, the selectable marker or tag is a polynucleotide barcode or unique molecular identifier (UMI).

It will be appreciated that the polynucleotide encoding such selectable markers or tags can be incorporated into a polynucleotide encoding one or more components of the genetic modifying system (or other polynucleotide) described herein in an appropriate manner to allow expression of the selectable marker or tag. Such techniques and methods are described elsewhere herein and will be instantly appreciated by one of ordinary skill in the art in view of this disclosure. Many such selectable markers and tags are generally known in the art and are intended to be within the scope of this disclosure.

Suitable selectable markers and tags include, but are not limited to, affinity tags, such as chitin binding protein (CBP), maltose binding protein (MBP), glutathione-S-transferase (GST), poly(His) tag; solubilization tags such as thioredoxin (TRX) and poly(NANP), MBP, and GST; chromatography tags such as those consisting of polyanionic amino acids, such as FLAG-tag; epitope tags such as V5-tag, Myc-tag, HA-tag and NE-tag; protein tags that can allow specific enzymatic modification (such as biotinylation by biotin ligase) or chemical modification (such as reaction with FlAsH-EDT2 for fluorescence imaging), DNA and/or RNA segments that contain restriction enzyme or other enzyme cleavage sites; DNA segments that encode products that provide resistance against otherwise toxic compounds including antibiotics, such as, spectinomycin, ampicillin, kanamycin, tetracycline, Basta, neomycin phosphotransferase II (NEO), hygromycin phosphotransferase (HPT)) and the like; DNA and/or RNA segments that encode products that are otherwise lacking in the recipient cell (e.g., tRNA genes, auxotrophic markers); DNA and/or RNA segments that encode products which can be readily identified (e.g., phenotypic markers such as β-galactosidase, GUS; fluorescent proteins such as green fluorescent protein (GFP), cyan (CFP), yellow (YFP), red (RFP), luciferase, and cell surface proteins); polynucleotides that can generate one or more new primer sites for PCR (e.g., the juxtaposition of two DNA sequences not previously juxtaposed), DNA sequences not acted upon or acted upon by a restriction endonuclease or other DNA modifying enzyme, chemical, etc.; epitope tags (e.g., GFP, FLAG- and His-tags), and, DNA sequences that make a molecular barcode or unique molecular identifier (UMI), DNA sequences required for a specific modification (e.g., methylation) that allows its identification. Other suitable markers will be appreciated by those of skill in the art.

Selectable markers and tags can be operably linked to one or more components of the genetic modifying system (or other polypeptide) described herein via suitable linker, such as a glycine or glycine serine linkers as short as GS or GG up to (GGGGG)₃ (SEQ ID NO: 19) or (GGGGS)₃ (SEQ ID NO: 20). Other suitable linkers are described elsewhere herein.

### Targeting Moieties

The vector or vector system (or other polynucleotide) can include one or more polynucleotides that are or encode one or more targeting moieties. In some embodiments, the targeting moiety encoding polynucleotides can be included in the vector or vector system, such as a viral vector system, such that they are expressed within and/or on the virus particle(s) produced such that the virus particles can be targeted to specific cells, tissues, organs, etc. In some embodiments, the targeting moiety encoding polynucleotides can be included in the vector or vector system such that the genetic modifying system polynucleotide(s) and/or products expressed therefrom include the targeting moiety and can be targeted to specific cells, tissues, organs, etc. In some embodiments, such as non-viral carriers, the targeting moiety can be attached to the carrier (e.g., polymer, lipid, inorganic molecule etc.) and can be capable of targeting the carrier and any attached or associated genetic modifying system polynucleotide(s) to specific cells, tissues, organs, etc. In some embodiments, the targeting moieties can target integrins on cell surfaces. Optionally, the binding affinity of the targeting moiety is in the range of 1 nM to 1 μM.

Exemplary targeting moieties that can be included are described elsewhere herein. See description related to “Targeted Delivery” and/or “Responsive Delivery” herein.

### Codon Optimization

As described elsewhere herein, the engineered polynucleotide of present disclosure described herein can be codon optimized. In some embodiments, one or more polynucleotides contained in a vector (“vector polynucleotides”) described herein that are in addition to an optionally codon optimized polynucleotide encoding embodiments of the genetic modifying system described herein can be codon optimized. In general, codon optimization refers to a process of modifying a nucleic acid sequence for enhanced expression in the host cells of interest by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.orjp/codon/and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, PA), are also available. In some embodiments, one or more codons (e.g., 1, 2, 3, 4, 5, 10, 15, 20, 25, 50, or more, or all codons) in a sequence encoding a DNA/RNA-targeting Cas protein corresponds to the most frequently used codon for a particular amino acid. As to codon usage in yeast, reference is made to the online Yeast Genome database available at www.yeastgenome.org/community/codon_usage.shtml, or Codon selection in yeast, Bennetzen and Hall, J Biol Chem. 1982 Mar. 25; 257(6):3026-31. As to codon usage in plants including algae, reference is made to Codon usage in higher plants, green algae, and cyanobacteria, Campbell and Gowri, Plant Physiol. 1990 January; 92(1): 1-11.; as well as Codon usage inplantgenes, Murray et al, Nucleic Acids Res. 1989 Jan. 25; 17(2):477-98; or Selection on the codon bias of chloroplast and cyanelle genes in different plant and algal lineages, Morton B R, J Mol Evol. 1998 April; 46(4):449-59.

The engineered polynucleotide and/or vector or polynucleotide thereof can be codon optimized for expression in a specific cell-type, tissue type, organ type, and/or subject type, including but not limited to a crustacean (e.g., shrimp), fish, insect, mammalian (e.g., human or non-human mammal), or plant cell. In some embodiments, a codon optimized sequence is a sequence optimized for expression in a eukaryote, e.g., a crustacean, or for another eukaryote, such as another animal (e.g., a mammal or plant). Such codon optimized sequences are within the ambit of the ordinary skilled artisan in view of the description herein. In some embodiments, the polynucleotide is codon optimized for a specific cell type. Such cell types can include, but are not limited to, epithelial cells (including skin cells, cells lining the gastrointestinal tract, cells lining other hollow organs), nerve cells (nerves, brain cells, spinal column cells, nerve support cells (e.g., astrocytes, glial cells, Schwann cells etc.), muscle cells (e.g., cardiac muscle, smooth muscle cells, and skeletal muscle cells), connective tissue cells (fat and other soft tissue padding cells, bone cells, tendon cells, cartilage cells), blood cells, stem cells (including embryonic stem cells, primordial germ cells, primordial germ cell like cells, pluripotent stem cells, totipotent stem cells, blastocysts, etc.) and other progenitor cells, immune system cells, germ cells, and combinations thereof. Such codon optimized sequences are within the ambit of the ordinary skilled artisan in view of the description herein. In some embodiments, the polynucleotide is codon optimized for a specific tissue type. Such tissue types can include, but are not limited to, muscle tissue, connective tissue, connective tissue, nervous tissue, and epithelial tissue. Such codon optimized sequences are within the ambit of the ordinary skilled artisan in view of the description herein. In some embodiments, the polynucleotide is codon optimized for a specific organ. Such organs include, but are not limited to, muscles, skin, intestines, liver, spleen, brain, lungs, stomach, heart, kidneys, gallbladder, pancreas, bladder, thyroid, bone, blood vessels, blood, and combinations thereof. Such codon optimized sequences are within the ambit of the ordinary skilled artisan in view of the description herein.

### Vector Construction

The vectors described herein can be constructed using any suitable process or technique. In some embodiments, one or more suitable recombination and/or cloning methods or techniques can be used to the vector(s) described herein. Suitable recombination and/or cloning techniques and/or methods can include, but not limited to, those described in U.S. Patent Publication No. US 2004/0171156 A1. Other suitable methods and techniques are described elsewhere herein.

Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989). Any of the techniques and/or methods can be used and/or adapted for constructing an AAV or other vectors described herein. nAAV vectors are discussed elsewhere herein.

In some embodiments, a vector comprises one or more insertion sites, such as a restriction endonuclease recognition sequence (also referred to as a “cloning site”). In some embodiments, one or more insertion sites (e.g., about or more than about 1, 2, 3, 4, 5, 6, 7, 8, 9, 10, or more insertion sites) are located upstream and/or downstream of one or more sequence elements of one or more vectors. When multiple different guide polynucleotides are used, a single expression construct may be used to target nucleic acid-targeting activity to multiple different, corresponding target sequences within a cell. For example, a single vector may comprise about or more than about 1, 2, 3, 4, 5, 6, 7, 8, 9, 10, 15, 20, or more guide s polynucleotides. In some embodiments, about or more than about 1, 2, 3, 4, 5, 6, 7, 8, 9, 10, or more such guide-polynucleotide-containing vectors may be provided, and optionally delivered to a cell.

### Viral Vectors

In some embodiments, the vector is a viral vector. The term of art “viral vector” and as used herein in this context refers to polynucleotide based vectors that contain one or more elements from or based upon one or more elements of a virus that can be capable of expressing and packaging a polynucleotide, such as a genetic modifying system polynucleotide of the present invention, into a virus particle and producing said virus particle when used alone or with one or more other viral vectors (such as in a viral vector system). Viral vectors and systems thereof can be used for producing viral particles for delivery of and/or expression of one or more components of the genetic modifying system described herein. The viral vector can be part of a viral vector system involving multiple vectors. In some embodiments, systems incorporating multiple viral vectors can increase the safety of these systems. Suitable viral vectors can include retroviral-based vectors, lentiviral-based vectors, adenoviral-based vectors, adeno associated vectors, helper-dependent adenoviral (HdAd) vectors, hybrid adenoviral vectors, herpes simplex virus-based vectors, poxvirus-based vectors, and Epstein-Barr virus-based vectors. Other embodiments of viral vectors and viral particles produce therefrom are described elsewhere herein. In some embodiments, the viral vectors are configured to produce replication incompetent viral particles for improved safety of these systems.

In certain embodiments, the virus structural component, which can be encoded by one or more polynucleotides in a viral vector or vector system, comprises one or more capsid proteins including an entire capsid. In certain embodiments, such as wherein a viral capsid comprises multiple copies of different proteins, the delivery system can provide one or more of the same protein or a mixture of such proteins. For example, AAV comprises 3 capsid proteins, VP1, VP2, and VP3, thus delivery systems of the invention can comprise one or more of VP1, and/or one or more of VP2, and/or one or more of VP3. Accordingly, the present invention is applicable to a virus within the family Adenoviridae, such as Atadenovirus, e.g., Ovine atadenovirus D, Aviadenovirus, e.g., Fowl aviadenovirus A, Ichtadenovirus, e.g., Sturgeon ichtadenovirus A, Mastadenovirus (which includes adenoviruses such as all human adenoviruses), e.g., Human mastadenovirus C, and Siadenovirus, e.g., Frog siadenovirus A. Thus, a virus of within the family Adenoviridae is contemplated as within the invention with discussion herein as to adenovirus applicable to other family members. Target-specific AAV capsid variants can be used or selected. Non-limiting examples include capsid variants selected to bind to chronic myelogenous leukemia cells, human CD34 PBPC cells, breast cancer cells, cells of lung, heart, dermal fibroblasts, melanoma cells, stem cell, glioblastoma cells, coronary artery endothelial cells and keratinocytes. See, e.g., Buning et al, 2015, Current Opinion in Pharmacology 24, 94-104. From teachings herein and knowledge in the art as to modifications of adenovirus (see, e.g., U.S. Pat. Nos. 9,410,129, 7,344,872, 7,256,036, 6,911,199, 6,740,525; Matthews, “Capsid-Incorporation of Antigens into Adenovirus Capsid Proteins for a Vaccine Approach,” Mol Pharm, 8(1): 3-11 (2011)), as well as regarding modifications of AAV, the skilled person can readily obtain a modified adenovirus that has a large payload protein or a CRISPR-protein, despite that heretofore it was not expected that such a large protein could be provided on an adenovirus. And as to the viruses related to adenovirus mentioned herein, as well as to the viruses related to AAV mentioned elsewhere herein, the teachings herein as to modifying adenovirus and AAV, respectively, can be applied to those viruses without undue experimentation from this disclosure and the knowledge in the art.

In some embodiments, the viral vector is configured such that when the cargo is packaged the cargo(s) is external to the capsid or virus particle. In the sense that it is not inside the capsid (enveloped or encompassed with the capsid) but is externally exposed so that it can contact the target genomic DNA. In some embodiments, the viral vector is configured such that all the cargo(s) are contained within the capsid after packaging.

### Split Viral Vector Systems

When the viral vector or vector system (be it a retroviral (e.g., AAV) or lentiviral vector) is designed so as to position the cargo(s) at the internal surface of the capsid once formed, the cargo(s) will fill most or all of internal volume of the capsid. In other embodiments, the cargo(s) may be modified or divided so as to occupy a less of the capsid internal volume. Accordingly, in certain embodiments, cargo (e.g., a gene or other nucleic acid) can be divided in two portions, which can be packaged in separate viral or viral like particles. In certain embodiments, by splitting the cargo in two (or more) portions, space is made available to link one or more heterologous protein domains or other protein portions encoded by the nucleic acid cargo. Such systems can be referred to as “split vector systems”. When the concept is applied to a vector system, it thus describes putting pieces of the split proteins on different vectors thus reducing the payload of any one vector. This approach can facilitate delivery of systems where the total system size is close to or exceeds the packaging capacity of the vector. This is independent of any regulation of e.g., a protein that can be achieved with a split system or split protein design.

Split exogenous proteins whose encoding polynucleotides can be incorporated into the viral or other vectors described herein are set forth elsewhere herein and in documents incorporated herein by reference in further detail herein. In certain embodiments, each part of a split protein are attached to a member of a specific binding pair, and when bound with each other, the members of the specific binding pair maintain the parts of the spit protein in proximity. In certain embodiments, each part of a split protein is associated with an inducible binding pair. An inducible binding pair is one which is capable of being switched “on” or “off” by a protein or small molecule that binds to both members of the inducible binding pair. In general, some proteins may preferably split between domains, leaving domains intact.

### Retroviral and Lentiviral Vectors

Retroviral vectors can be composed of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Suitable retroviral vectors for the delivery of a cargo (e.g., a exogenous polynucleotide) can include, but are not limited to, those vectors based upon murine leukemia virus (MuLV), gibbon ape leukemia virus (GaLV), Simian immunodeficiency virus (SIV), human immunodeficiency virus (HIV), equine infections anemia (EIA), and combinations thereof (see, e.g., Buchscher et al., J. Virol. 66:2731-2739 (1992); Johann et al., J. Virol. 66:1635-1640 (1992); Sommnerfelt et al., Virol. 176:58-59 (1990); Wilson et al., J. Virol. 63:2374-2378 (1989); Miller et al., J. Virol. 65:2220-2224 (1991); WO1994026877). Other exemplary retroviral vectors are described elsewhere herein.

The tropism of a retrovirus can be altered by incorporating foreign envelope proteins, expanding the potential target population of target cells. Lentiviral vectors are retroviral vectors that are able to transduce or infect non-dividing cells and are described in greater detail elsewhere herein. A retrovirus can also be engineered to allow for conditional expression of the inserted transgene, such that only certain cell types are infected by the lentivirus.

Lentiviruses are complex retroviruses that have the ability to infect and express their genes in both mitotic and post-mitotic cells. Advantages of using a lentiviral approach can include the ability to transduce or infect non-dividing cells and their ability to typically produce high viral titers, which can increase efficiency or efficacy of production and delivery. Exemplary lentiviral vectors include, but are not limited to, human immunodeficiency virus (HIV)-based lentiviral vectors, feline immunodeficiency virus (FIV)-based lentiviral vectors, simian immunodeficiency virus (SIV)-based lentiviral vectors, Moloney Murine Leukaemia Virus (Mo-MLV), Visna.maedi virus (VMV)-based lentiviral vector, carpine arthritis-encephalitis virus (CAEV)-based lentiviral vector, bovine immune deficiency virus (BIV)-based lentiviral vector, and Equine infectious anemia (EIAV)-based lentiviral vector. In some embodiments, an HIV-based lentiviral vector system can be used. In some embodiments, a FIV-based lentiviral vector system can be used.

In some embodiments, the lentiviral vector is an EIAV-based lentiviral vector or vector system. See e.g., Balagaan, J Gene Med 2006; 8: 275-285; Binley et al., HUMAN GENE THERAPY 23:980-991 (September 2012)), which can be modified for use with the present disclosure.

In some embodiments, the lentiviral vector or vector system thereof can be a first-generation lentiviral vector or vector system thereof. First-generation lentiviral vectors can contain a large portion of the lentivirus genome, including the gag and pol genes, other additional viral proteins (e.g., VSV-G) and other accessory genes (e.g., vif, vprm vpu, nef, and combinations thereof), regulatory genes (e.g., tat and/or rev) as well as the gene of interest between the LTRs. First generation lentiviral vectors can result in the production of virus particles that can be capable of replication in vivo, which may not be appropriate for some instances or applications.

In some embodiments, the lentiviral vector or vector system thereof can be a second-generation lentiviral vector or vector system thereof. Second-generation lentiviral vectors do not contain one or more accessory virulence factors and do not contain all components necessary for virus particle production on the same lentiviral vector. This can result in the production of a replication-incompetent virus particle and thus increase the safety of these systems over first-generation lentiviral vectors. In some embodiments, the second-generation vector lacks one or more accessory virulence factors (e.g., vif, vprm, vpu, nef, and combinations thereof). Unlike the first-generation lentiviral vectors, no single second generation lentiviral vector includes all features necessary to express and package a polynucleotide into a virus particle. In some embodiments, the envelope and packaging components are split between two different vectors with the gag, pol, rev, and tat genes being contained on one vector and the envelope protein (e.g., VSV-G) are contained on a second vector. The gene of interest, its promoter, and LTRs can be included on a third vector that can be used in conjunction with the other two vectors (packaging and envelope vectors) to generate a replication-incompetent virus particle.

In some embodiments, the lentiviral vector or vector system thereof can be a third-generation lentiviral vector or vector system thereof. Third-generation lentiviral vectors and vector systems thereof have increased safety over first- and second-generation lentiviral vectors and systems thereof because, for example, the various components of the viral genome are split between two or more different vectors but used together in vitro to make virus particles, they can lack the tat gene (when a constitutively active promoter is included up-stream of the LTRs), and they can include one or more deletions in the 3′LTR to create self-inactivating (SIN) vectors having disrupted promoter/enhancer activity of the LTR. In some embodiments, a third-generation lentiviral vector system can include (i) a vector plasmid that contains the polynucleotide of interest and upstream promoter that are flanked by the 5′ and 3′ LTRs, which can optionally include one or more deletions present in one or both of the LTRs to render the vector self-inactivating; (ii) a “packaging vector(s)” that can contain one or more genes involved in packaging a polynucleotide into a virus particle that is produced by the system (e.g. gag, pol, and rev) and upstream regulatory sequences (e.g. promoter(s)) to drive expression of the features present on the packaging vector, and (iii) an “envelope vector” that contains one or more envelope protein genes and upstream promoters. In certain embodiments, the third-generation lentiviral vector system can include at least two packaging vectors, with the gag-pol being present on a different vector than the rev gene.

In some embodiments, self-inactivating lentiviral vectors with an siRNA targeting a common exon shared by HIV tat/rev, a nucleolar-localizing TAR decoy, and an anti-CCR5-specific hammerhead ribozyme (see, e.g., DiGiusto et al. (2010) Sci Transl Med 2:36ra43) can be used/and or adapted to deliver a genetic modifying system or exogenous polynucleotide of the present disclosure.

In some embodiments, the pseudotype and infectivity or tropism of a lentivirus particle can be tuned by altering the type of envelope protein(s) included in the lentiviral vector or system thereof. As used herein, an “envelope protein” or “outer protein” means a protein exposed at the surface of a viral particle that is not a capsid protein. For example, envelope or outer proteins typically comprise proteins embedded in the envelope of the virus. In some embodiments, a lentiviral vector or vector system thereof can include a VSV-G envelope protein. VSV-G mediates viral attachment to an LDL receptor (LDLR) or an LDLR family member present on a host cell, which triggers endocytosis of the viral particle by the host cell. Because LDLR is expressed by a wide variety of cells, viral particles expressing the VSV-G envelope protein can infect or transduce a wide variety of cell types. Other suitable envelope proteins can be incorporated based on the host cell that a user desires to be infected by a virus particle produced from a lentiviral vector or system thereof described herein and can include, but are not limited to, feline endogenous virus envelope protein (RD 114) (see e.g., Hanawa et al. Molec. Ther. 2002 5(3) 242-251), modified Sindbis virus envelope proteins (see e.g., Morizono et al. 2010. J. Virol. 84(14) 6923-6934; Morizono et al. 2001. J. Virol. 75:8016-8020; Morizono et al. 2009. J. Gene Med. 11:549-558; Morizono et al. 2006 Virology 355:71-81; Morizono et al J. Gene Med. 11:655-663, Morizono et al. 2005 Nat. Med. 11:346-352), baboon retroviral envelope protein (see e.g., Girard-Gagnepain et al. 2014. Blood. 124: 1221-1231); Tupaia paramyxovirus glycoproteins (see e.g., Enkirch T. et al., 2013. Gene Ther. 20:16-23); measles virus glycoproteins (see e.g., Funke et al. 2008. Molec. Ther. 16(8): 1427-1436), rabies virus envelope proteins, MLV envelope proteins, Ebola envelope proteins, baculovirus envelope proteins, filovirus envelope proteins, hepatitis E1 and E2 envelope proteins, gp41 and gp120 of HIV, hemagglutinin, neuraminidase, M2 proteins of influenza virus, and combinations thereof.

In some embodiments, the tropism of the resulting lentiviral particle can be tuned by incorporating cell targeting peptides into a lentiviral vector such that the cell targeting peptides are expressed on the surface of the resulting lentiviral particle. In some embodiments, a lentiviral vector can contain an envelope protein that is fused to a cell targeting protein (see e.g., Buchholz et al. 2015. Trends Biotechnol. 33:777-790; Bender et al. 2016. PLoS Pathog. 12(e1005461); and Friedrich et al. 2013. Mol. Ther. 2013. 21: 849-859).

In some embodiments, a split-intein-mediated approach to target lentiviral particles to a specific cell type can be used (see e.g., Chamoun-Emaneulli et al. 2015. Biotechnol. Bioeng. 112:2611-2617, Ramirez et al. 2013. Protein. Eng. Des. Sel. 26:215-233. In these embodiments, a lentiviral vector can contain one half of a splicing-deficient variant of the naturally split intein from Nostoc punctiforme fused to a cell targeting peptide and the same or different lentiviral vector can contain the other half of the split intein fused to an envelope protein, such as a binding-deficient, fusion-competent virus envelope protein. This can result in production of a virus particle from the lentiviral vector or vector system that includes a split intein that can function as a molecular Velcro linker to link the cell-binding protein to the pseudotyped lentivirus particle. This approach can be advantageous for use where surface-incompatibilities can restrict the use of, e.g., cell targeting peptides.

In some embodiments, a covalent-bond-forming protein-peptide pair can be incorporated into one or more of the lentiviral vectors described herein to conjugate a cell targeting peptide to the virus particle (see e.g., Kasaraneni et al. 2018. Sci. Reports (8) No. 10990). In some embodiments, a lentiviral vector can include an N-terminal PDZ domain of InaD protein (PDZ1) and its pentapeptide ligand (TEFCA (SEQ ID NO: 21)) from NorpA, which can conjugate the cell targeting peptide to the virus particle via a covalent bond (e.g., a disulfide bond). In some embodiments, the PDZ1 protein can be fused to an envelope protein, which can optionally be binding deficient and/or fusion competent virus envelope protein and included in a lentiviral vector. In some embodiments, the TEFCA (SEQ ID NO: 21) can be fused to a cell targeting peptide and the TEFCA-CPT fusion construct can be incorporated into the same or a different lentiviral vector as the PDZ1-envenlope protein construct. During virus production, specific interaction between the PDZ1 and TEFCA (SEQ ID NO: 21) facilitates producing virus particles covalently functionalized with the cell targeting peptide and thus capable of targeting a specific cell-type based upon a specific interaction between the cell targeting peptide and cells expressing its binding partner. This approach can be advantageous for use where surface-incompatibilities can restrict the use of, e.g., cell targeting peptides.

Various exemplary lentiviral vectors are described in e.g., US Patent Publication No. 20120295960, 20060281180, 20090007284, US20110117189; US20090017543; US20070054961, US20100317109, US20110293571; US20110293571, US20040013648, US20070025970, US20090111106, and U.S. Pat. Nos. 7,259,015, 7,303,910 and 7,351,585. Any of these systems can be used or adapted to deliver a genetic modifying system polynucleotide or other exogenous polynucleotide of the present disclosure.

In some embodiments, a lentiviral vector system can include one or more transfer plasmids. Transfer plasmids can be generated from various other vector backbones and can include one or more features that can work with other retroviral and/or lentiviral vectors in the system that can, for example, improve safety of the vector and/or vector system, increase virial titers, and/or increase or otherwise enhance expression of the desired insert to be expressed and/or packaged into the viral particle. Suitable features that can be included in a transfer plasmid can include, but are not limited to, 5′LTR, 3′LTR, SIN/LTR, origin of replication (Ori), selectable marker genes (e.g., antibiotic resistance genes), Psi (Ψ), RRE (rev response element), cPPT (central polypurine tract), promoters, WPRE (woodchuck hepatitis post-transcriptional regulatory element), SV40 polyadenylation signal, pUC origin, SV40 origin, F1 origin, and combinations thereof.

In another embodiment, the viral vector is a Cocal vesiculovirus envelope pseudotyped retroviral or lentiviral vector particles are contemplated (see, e.g., US Patent Publication No. 20120164118). Cocal virus is in the Vesiculovirus genus and is a causative agent of vesicular stomatitis in mammals, and as such vectors based on this virus can be used to deliver cells to a wide variety of animals, including insects, cattle, and horses (see e.g., Jonkers et al., Am. J. Vet. Res. 25:236-242 (1964) and Travassos da Rosa et al., Am. J. Tropical Med. & Hygiene 33:999-1006 (1984)). In some embodiments, Cocal vesiculovirus envelope pseudotyped retroviral vector particles may include for example, lentiviral, alpharetroviral, betaretroviral, gammaretroviral, deltaretroviral, and epsilonretroviral vector particles that may comprise retroviral Gag, Pol, and/or one or more accessory protein(s) and a Cocal vesiculovirus envelope protein. In certain embodiments of these embodiments, the Gag, Pol, and accessory proteins are lentiviral and/or gammaretroviral. In some embodiments, a retroviral vector can contain encoding polypeptides for one or more Cocal vesiculovirus envelope proteins such that the resulting viral or pseudoviral particles are Cocal vesiculovirus envelope pseudotyped.

### Adenoviral Vectors, Helper-Dependent Adenoviral Vectors, and Hybrid Adenoviral Vectors

In some embodiments, the vector can be an adenoviral vector. In some embodiments, the adenoviral vector can include elements such that the virus particle produced using the vector or system thereof can be any suitable serotype, such as serotype 2, 5, 8, 9, and others. In some embodiments, the polynucleotide to be delivered via the adenoviral particle can be up to about 8 kb. Thus, in some embodiments, an adenoviral vector can include a DNA polynucleotide to be delivered that can range in size from about 0.001 kb to about 8 kb. Adenoviral vectors have been used successfully in several contexts (see e.g., Teramato et al. 2000. Lancet. 355:1911-1912; Lai et al. 2002. DNA Cell. Biol. 21:895-913; Flotte et al., 1996. Hum. Gene. Ther. 7:1145-1159; and Kay et al. 2000. Nat. Genet. 24:257-261.

In some embodiments the vector can be a helper-dependent adenoviral vector or system thereof. These are also referred to in the art as “gutless” or “gutted” vectors and are a modified generation of adenoviral vectors (see e.g., Thrasher et al. 2006. Nature. 443:E5-7). In certain embodiments of the helper-dependent adenoviral vector system one vector (the helper) can contain all the viral genes required for replication but contains a conditional gene defect in the packaging domain. The second vector of the system can contain only the ends of the viral genome, one or more exogenous polynucleotides, and the native packaging recognition signal, which can allow selective packaged release from the cells (see e.g., Cideciyan et al. 2009. N Engl J Med. 361:725-727). Helper-dependent adenoviral vector systems have been successful for gene delivery in several contexts (see e.g., Simonelli et al. 2010. J Am Soc Gene Ther. 18:643-650; Cideciyan et al. 2009. N Engl J Med. 361:725-727; Crane et al. 2012. Gene Ther. 19(4):443-452; Alba et al. 2005. Gene Ther. 12:18-S27; Croyle et al. 2005. Gene Ther. 12:579-587; Amalfitano et al. 1998. J. Virol. 72:926-933; and Morral et al. 1999. PNAS. 96:12816-12821). The techniques and vectors described in these publications can be adapted for inclusion and delivery of one or more genes or other polynucleotides. In some embodiments, the gene or other polynucleotide to be delivered via the viral particle produced from a helper-dependent adenoviral vector or system thereof can be up to about 37 kb. Thus, in some embodiments, an adenoviral vector can include a DNA polynucleotide to be delivered that can range in size from about 0.001 kb to about 37 kb (see e.g., Rosewell et al. 2011. J. Genet. Syndr. Gene Ther. Suppl. 5:001).

In some embodiments, the vector is a hybrid-adenoviral vector or system thereof. Hybrid adenoviral vectors are composed of the high transduction efficiency of a gene-deleted adenoviral vector and the long-term genome-integrating potential of adeno-associated, retroviruses, lentivirus, and transposon based-gene transfer. In some embodiments, such hybrid vector systems can result in stable transduction and limited integration site. See e.g., Balague et al. 2000. Blood. 95:820-828; Morral et al. 1998. Hum. Gene Ther. 9:2709-2716; Kubo and Mitani. 2003. J. Virol. 77(5): 2964-2971; Zhang et al. 2013. PloS One. 8(10) e76771; and Cooney et al. 2015. Mol. Ther. 23(4):667-674), whose techniques and vectors described therein can be modified and adapted for use to deliver and/or express a polynucleotide or system of the present description. In some embodiments, a hybrid-adenoviral vector can include one or more features of a retrovirus and/or an adeno-associated virus. In some embodiments the hybrid-adenoviral vector can include one or more features of a spuma retrovirus or foamy virus (FV). See e.g., Ehrhardt et al. 2007. Mol. Ther. 15:146-156 and Liu et al. 2007. Mol. Ther. 15:1834-1841, whose techniques and vectors described therein can be modified and adapted for use to deliver and/or express a polynucleotide or system of the present description. Advantages of using one or more features from the FVs in the hybrid-adenoviral vector or system thereof can include the ability of the viral particles produced therefrom to infect a broad range of cells, a large packaging capacity as compared to other retroviruses, and the ability to persist in quiescent (non-dividing) cells. See also e.g., Ehrhardt et al. 2007. Mol. Ther. 156:146-156 and Shuji et al. 2011. Mol. Ther. 19:76-82, whose techniques and vectors described therein can be modified and adapted for use to deliver and/or express a polynucleotide or system of the present description.

### Adeno Associated Viral (AAV) Vectors

In an embodiment, the vector can be an adeno-associated virus (AAV) vector. See, e.g., West et al., Virology 160:38-47 (1987); U.S. Pat. No. 4,797,368; WO 93/24641; Kotin, Human Gene Therapy 5:793-801 (1994); and Muzyczka, J. Clin. Invest. 94:1351 (1994). Although similar to adenoviral vectors in some of their features, AAVs have some deficiency in their replication and/or pathogenicity and thus can be safer that adenoviral vectors. In some embodiments the AAV can integrate into a specific site on chromosome 19 of a human cell with no observable side effects. In some embodiments, the capacity of the AAV vector, system thereof, and/or AAV particles can be up to about 4.7 kb.

The AAV vector or system thereof can include one or more regulatory molecules. In some embodiments the regulatory molecules can be promoters, enhancers, repressors and the like, which are described in greater detail elsewhere herein. In some embodiments, the AAV vector or system thereof can include one or more polynucleotides that can encode one or more regulatory proteins. In some embodiments, the one or more regulatory proteins can be selected from Rep78, Rep68, Rep52, Rep40, variants thereof, and combinations thereof.

The AAV vector or system thereof can include one or more polynucleotides that can encode one or more capsid proteins. The capsid proteins can be selected from VP1, VP2, VP3, and combinations thereof. The capsid proteins can be capable of assembling into a protein shell of the AAV virus particle. In some embodiments, the AAV capsid can contain 60 capsid proteins. In some embodiments, the ratio of VP1:VP2:VP3 in a capsid can be about 1:1:10.

In some embodiments, the AAV vector or system thereof can include one or more adenovirus helper factors or polynucleotides that can encode one or more adenovirus helper factors. Such adenovirus helper factors can include, but are not limited, E1A, E1B, E2A, E40RF6, and VA RNAs. In some embodiments, a producing host cell line expresses one or more of the adenovirus helper factors.

The AAV vector or system thereof can be configured to produce AAV particles having a specific serotype.

AAV particles, packaging polynucleotides encoding compositions of the present disclosure may comprise or be derived from any natural or recombinant AAV serotype. According to the present disclosure, the AAV particles may utilize or be based on a serotype selected from any of the following serotypes, and variants thereof including but not limited to AAV1, AAV10, AAV106.1/hu.37, AAV11, AAV114.3/hu.40, AAV12, AAV127.2/hu.41, AAV127.5/hu.42, AAV128.1/hu.43, AAV128.3/hu.44, AAV130.4/hu.48, AAV145.1/hu.53, AAV145.5/hu.54, AAV145.6/hu.55, AAV16.12/hu.11, AAV16.3, AAV16.8/hu.10, AAV161.10/hu.60, AAV161.6/hu.61, AAV1-7/rh.48, AAV1-8/rh.49, AAV2, AAV2.5T, AAV2-15/rh.62, AAV223.1, AAV223.2, AAV223.4, AAV223.5, AAV223.6, AAV223.7, AAV2-3/rh.61, AAV24.1, AAV2-4/rh.50, AAV2-5/rh.51, AAV27.3, AAV29.3/bb.1, AAV29.5/bb.2, AAV2G9, AAV-2-pre-miRNA-101, AAV3, AAV3.1/hu.6, AAV3.1/hu.9, AAV3-11/rh.53, AAV3-3, AAV33.12/hu.17, AAV33.4/hu.15, AAV33.8/hu.16, AAV3-9/rh.52, AAV3a, AAV3b, AAV4, AAV4-19/rh.55, AAV42.12, AAV42-10, AAV42-11, AAV42-12, AAV42-13, AAV42-15, AAV42-1b, AAV42-2, AAV42-3a, AAV42-3b, AAV42-4, AAV42-5a, AAV42-5b, AAV42-6b, AAV42-8, AAV42-aa, AAV43-1, AAV43-12, AAV43-20, AAV43-21, AAV43-23, AAV43-25, AAV43-5, AAV4-4, AAV44.1, AAV44.2, AAV44.5, AAV46.2/hu.28, AAV46.6/hu.29, AAV4-8/r11.64, AAV4-8/rh.64, AAV4-9/rh.54, AAV5, AAV52.1/hu.20, AAV52/hu.19, AAV5-22/rh.58, AAV5-3/rh.57, AAV54.1/hu.21, AAV54.2/hu.22, AAV54.4R/hu.27, AAV54.5/hu.23, AAV54.7/hu.24, AAV58.2/hu.25, AAV6, AAV6.1, AAV6.1.2, AAV6.2, AAV7, AAV7.2, AAV7.3/hu.7, AAV8, AAV-8b, AAV-8h, AAV9, AAV9.11, AAV9.13, AAV9.16, AAV9.24, AAV9.45, AAV9.47, AAV9.61, AAV9.68, AAV9.84, AAV9.9, AAVA3.3, AAVA3.4, AAVA3.5, AAVA3.7, AAV-b, AAVC1, AAVC2, AAVC5, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAV-DJ, AAV-DJ8, AAVF3, AAVF5, AAV-h, AAVH-1/hu.1, AAVH2, AAVH-5/hu.3, AAVH6, AAVhE1.1, AAVhER1.14, AAVhEr1.16, AAVhEr1.18, AAVhER1.23, AAVhEr1.35, AAVhEr1.36, AAVhEr1.5, AAVhEr1.7, AAVhEr1.8, AAVhEr2.16, AAVhEr2.29, AAVhEr2.30, AAVhEr2.31, AAVhEr2.36, AAVhEr2.4, AAVhEr3.1, AAVhu.1, AAVhu.10, AAVhu.11, AAVhu.11, AAVhu.12, AAVhu.13, AAVhu.14/9, AAVhu.15, AAVhu.16, AAVhu.17, AAVhu.18, AAVhu.19, AAVhu.2, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.3, AAVhu.31, AAVhu.32, AAVhu.34, AAVhu.35, AAVhu.37, AAVhu.39, AAVhu.4, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.5, AAVhu.51, AAVhu.52, AAVhu.53, AAVhu.54, AAVhu.55, AAVhu.56, AAVhu.57, AAVhu.58, AAVhu.6, AAVhu.60, AAVhu.61, AAVhu.63, AAVhu.64, AAVhu.66, AAVhu.67, AAVhu.7, AAVhu.8, AAVhu.9, AAVhu.t 19, AAVLG-10/rh.40, AAVLG-4/rh.38, AAVLG-9/hu.39, AAVLG-9/hu.39, AAV-LK01, AAV-LK02, AAVLK03, AAV-LK03, AAV-LK04, AAV-LK05, AAV-LK06, AAV-LK07, AAV-LK08, AAV-LK09, AAV-LK10, AAV-LK 11, AAV-LK12, AAV-LK13, AAV-LK14, AAV-LK15, AAV-LK17, AAV-LK18, AAV-LK19, AAVN721-8/rh.43, AAV-PAEC, AAV-PAEC11, AAV-PAEC12, AAV-PAEC2, AAV-PAEC4, AAV-PAEC6, AAV-PAEC7, AAV-PAEC8, AAVpi.1, AAVpi.2, AAVpi.3, AAVrh.10, AAVrh.12, AAVrh.13, AAVrh.13R, AAVrh.14, AAVrh.17, AAVrh.18, AAVrh.19, AAVrh.2, AAVrh.20, AAVrh.21, AAVrh.22, AAVrh.23, AAVrh.24, AAVrh.25, AAVrh.2R, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.43, AAVrh.44, AAVrh.45, AAVrh.46, AAVrh.47, AAVrh.48, AAVrh.48, AAVrh.48.1, AAVrh.48.1.2, AAVrh.48.2, AAVrh.49, AAVrh.50, AAVrh.51, AAVrh.52, AAVrh.53, AAVrh.54, AAVrh.55, AAVrh.56, AAVrh.57, AAVrh.58, AAVrh.59, AAVrh.60, AAVrh.61, AAVrh.62, AAVrh.64, AAVrh.64R1, AAVrh.64R2, AAVrh.65, AAVrh.67, AAVrh.68, AAVrh.69, AAVrh.70, AAVrh.72, AAVrh.73, AAVrh.74, AAVrh.8, AAVrh.8R, AAVrh8R, AAVrh8R A586R mutant, AAVrh8R R533A mutant, BAAV, BNP61 AAV, BNP62 AAV, BNP63 AAV, bovine AAV, caprine AAV, Japanese AAV 10, true type AAV (ttAAV), UPENN AAV 10, AAV-LK16, AAAV, AAV Shuffle 100-1, AAV Shuffle 100-2, AAV Shuffle 100-3, AAV Shuffle 100-7, AAV Shuffle 10-2, AAV Shuffle 10-6, AAV Shuffle 10-8, AAV SM 100-10, AAV SM 100-3, AAV SM 10-1, AAV SM 10-2, and/or AAV SM 10-8.

In some embodiments s, the AAV serotype may be, or have, a mutation in the AAV9 sequence as described by N Pulicherla et al. (Molecular Therapy 19(6):1070-1078 (2011)), such as but not limited to, AAV9.9, AAV9.11, AAV9.13, AAV9.16, AAV9.24, AAV9.45, AAV9.47, AAV9.61, AAV9.68, AAV9.84.

In some embodiments, the AAV serotype may be, or have, a sequence as described in U.S. Pat. No. 6,156,303, such as, but not limited to, AAV3B (SEQ ID NO: 1 and 10 of U.S. Pat. No. 6,156,303), AAV6 (SEQ ID NO: 2, 7 and 11 of U.S. Pat. No. 6,156,303), AAV2 (SEQ ID NO: 3 and 8 of U.S. Pat. No. 6,156,303), AAV3A (SEQ ID NO: 4 and 9, of U.S. Pat. No. 6,156,303), or derivatives thereof.

In some embodiments, the serotype may be AAVDJ or a variant thereof, such as AAVDJ8 (or AAV-DJ8), as described by Grimm et al. (Journal of Virology 82(12): 5887-5911 (2008). The amino acid sequence of AAVDJ8 may comprise two or more mutations in order to remove the heparin binding domain (HBD). As a non-limiting example, the AAV-DJ sequence described as SEQ ID NO: 1 in U.S. Pat. No. 7,588,772 may comprise two mutations: (1) R587Q where arginine (R; Arg) at amino acid 587 is changed to glutamine (Q; Gln) and (2) R590T where arginine (R; Arg) at amino acid 590 is changed to threonine (T; Thr). As another non-limiting example, may comprise three mutations: (1) K406R where lysine (K; Lys) at amino acid 406 is changed to arginine (R; Arg), (2) R587Q where arginine (R; Arg) at amino acid 587 is changed to glutamine (Q; Gln) and (3) R590T where arginine (R; Arg) at amino acid 590 is changed to threonine (T; Thr).

In some embodiments, the AAV serotype may be, or have, a sequence as described in International Publication No. WO2015121501, such as, but not limited to, true type AAV (ttAAV) (SEQ ID NO: 2 of WO2015121501), “UPenn AAV10” (SEQ ID NO: 8 of WO2015/121501), “Japanese AAV10” (SEQ ID NO: 9 of WO2015/121501), or variants thereof.

According to the present disclosure, AAV capsid serotype selection or use may be from a variety of species. In one example, the AAV may be an avian AAV (AAAV). The AAAV serotype may be, or have, a sequence as described in U.S. Pat. No. 9,238,800, such as, but not limited to, AAAV (SEQ ID NO: 1, 2, 4, 6, 8, 10, 12, and 14 of U.S. Pat. No. 9,238,800), or variants thereof.

In one example, the AAV may be a bovine AAV (BAAV). The BAAV serotype may be, or have, a sequence as described in U.S. Pat. No. 9,193,769, such as, but not limited to, BAAV (SEQ ID NO: 1 and 6 of U.S. Pat. No. 9,193,769), or variants thereof. The BAAV serotype may be or have a sequence as described in U.S. Pat. No. 7,427,396, such as, but not limited to, BAAV (SEQ ID NO: 5 and 6 of U.S. Pat. No. 7,427,396), or variants thereof.

In one example, the AAV may be a caprine AAV. The caprine AAV serotype may be, or have, a sequence as described in U.S. Pat. No. 7,427,396, such as, but not limited to, caprine AAV (SEQ ID NO: 3 of U.S. Pat. No. 7,427,396), or variants thereof.

In other examples the AAV may be engineered as a hybrid AAV from two or more parental serotypes. In one example, the AAV may be AAV2G9 which comprises sequences from AAV2 and AAV9. The AAV2G9 AAV serotype may be, or have, a sequence as described in United States Patent Publication No. US2016/0017005.

In one example, the AAV may be a serotype generated by the AAV9 capsid library with mutations in amino acids 390-627 (VP1 numbering) as described by Pulicherla et al. (Molecular Therapy 19(6):1070-1078 (2011). The serotype and corresponding nucleotide and amino acid substitutions may be, but is not limited to, AAV9.1 (G1594C; D532H), AAV6.2 (T1418A and T1436X; V473D and I479K), AAV9.3 (T1238A; F413Y), AAV9.4 (T1250C and A1617T; F417S), AAV9.5 (A1235G, A1314T, A1642G, C1760T; Q412R, T548A, A587V), AAV9.6 (T1231A; F411I), AAV9.9 (G1203A, G1785T; W595C), AAV9.10 (A1500G, T1676C; M559T), AAV9.11 (A1425T, A1702C, A1769T; T568P, Q590L), AAV9.13 (A1369C, A1720T; N457H, T574S), AAV9.14 (T1340A, T1362C, T1560C, G1713A; L447H), AAV9.16 (A1775T; Q592L), AAV9.24 (T1507C, T1521G; W503R), AAV9.26 (A1337G, A1769C; Y446C, Q590P), AAV9.33 (A1667C; D556A), AAV9.34 (A1534G, C1794T; N512D), AAV9.35 (A1289T, T1450A, C1494T, A1515T, C1794A, G1816A; Q430L, Y484N, N98K, V6061), AAV9.40 (A1694T, E565V), AAV9.41 (A1348T, T1362C; T450S), AAV9.44 (A1684C, A1701T, A1737G; N562H, K567N), AAV9.45 (A1492T, C1804T; N498Y, L602F), AAV9.46 (G1441C, T1525C, T1549G; G481R, W509R, L517V), 9.47 (G1241A, G1358A, A1669G, C1745T; S414N, G453D, K557E, T5821), AAV9.48 (C1445T, A1736T; P482L, Q579L), AAV9.50 (A1638T, C1683T, T1805A; Q546H, L602H), AAV9.53 (G1301A, A1405C, C1664T, G1811T; R134Q, S469R, A555V, G604V), AAV9.54 (C1531A, T1609A; L511I, L537M), AAV9.55 (T1605A; F535L), AAV9.58 (C1475T, C1579A; T492I, H527N), AAV.59 (T1336C; Y446H), AAV9.61 (A1493T; N498I), AAV9.64 (C1531A, A1617T; L511I), AAV9.65 (C1335T, T1530C, C1568A; A523D), AAV9.68 (C1510A; P504T), AAV9.80 (G1441A; G481R), AAV9.83 (C1402A, A1500T; P468T, E500D), AAV9.87 (T1464C, T1468C; S490P), AAV9.90 (A1196T; Y399F), AAV9.91 (T1316G, A1583T, C1782G, T1806C; L439R, K5281), AAV9.93 (A1273G, A1421G, A1638C, C1712T, G1732A, A1744T, A1832T; S425G, Q474R, Q546H, P571L, G578R, T582S, D611V), AAV9.94 (A1675T; M559L) and AAV9.95 (T1605A; F535L).

In one example, the AAV may be a serotype including at least one AAV capsid CD8+ T-cell epitope. As a non-limiting example, the serotype may be AAV1, AAV2 or AAV8.

In one example, the AAV may be a variant, such as PHP.A or PHP.B as described in Deverman. 2016. Nature Biotechnology. 34(2): 204-209.

AAV vector serotypes can be matched to target cell types. For example, the following exemplary cell types can be transduced by the indicated AAV serotypes among others.

In some embodiments, the serotype can be AAV-1, AAV-2, AAV-3, AAV-4, AAV-5, AAV-6, AAV-8, AAV-9 or any combinations thereof. In some embodiments, the AAV can be AAV1, AAV-2, AAV-5 or any combination thereof. One can select the AAV of the AAV with regard to the cells to be targeted; e.g., one can select AAV serotypes 1, 2, 5 or a hybrid capsid AAV-1, AAV-2, AAV-5 or any combination thereof for targeting brain and/or neuronal cells; and one can select AAV-4 for targeting cardiac tissue; and one can select AAV8 for delivery to the liver. Thus, in some embodiments, an AAV vector or system thereof capable of producing AAV particles capable of targeting the brain and/or neuronal cells can be configured to generate AAV particles having serotypes 1, 2, 5 or a hybrid capsid AAV-1, AAV-2, AAV-5 or any combination thereof. In some embodiments, an AAV vector or system thereof capable of producing AAV particles capable of targeting cardiac tissue can be configured to generate an AAV particle having an AAV-4 serotype. In some embodiments, an AAV vector or system thereof capable of producing AAV particles capable of targeting the liver can be configured to generate an AAV having an AAV-8 serotype. In some embodiments, the AAV vector is a hybrid AAV vector or system thereof. Hybrid AAVs are AAVs that include genomes with elements from one serotype that are packaged into a capsid derived from at least one different serotype. For example, if it is the rAAV2/5 that is to be produced, and if the production method is based on the helper-free, transient transfection method discussed above, the 1st plasmid and the 3rd plasmid (the adeno helper plasmid) will be the same as discussed for rAAV2 production. However, the second plasmid, the pRepCap will be different. In this plasmid, called pRep2/Cap5, the Rep gene is still derived from AAV2, while the Cap gene is derived from AAV5. The production scheme is the same as the above-mentioned approach for AAV2 production. The resulting rAAV is called rAAV2/5, in which the genome is based on recombinant AAV2, while the capsid is based on AAV5. It is assumed the cell or tissue-tropism displayed by this AAV2/5 hybrid virus should be the same as that of AAV5. A tabulation of certain AAV serotypes as to these cells can be found in Grimm, D. et al, J. Virol. 82: 5887-5911 (2008).

In some embodiments, the AAV vector or system thereof is configured as a “gutless” vector, similar to that described in connection with a retroviral vector. In some embodiments, the “gutless” AAV vector or system thereof can have the cis-acting viral DNA elements involved in genome amplification and packaging in linkage with the heterologous sequences of interest (e.g., the genetic modifying system polynucleotide(s)).

In some embodiments, the AAV vectors are produced in in insect cells, e.g., Spodoptera frugiperda Sf9 insect cells, grown in serum-free suspension culture. Serum-free insect cells can be purchased from commercial vendors, e.g., Sigma Aldrich (EX-CELL 405).

In some embodiments, an AAV vector or vector system can contain or consists essentially of one or more polynucleotides encoding one or more components of a genetic modifying system or other exogenous polynucleotide to be delivered to a cell. Specific cassette configuration for delivery of a genetic modifying system and/or other exogenous polynucleotide(s) will be appreciated by one of ordinary skill in the art in view of the description herein.

In some embodiments, one or more components of a genetic modifying system or other polypeptides and/or polynucleotides are associated with Adeno Associated Virus (AAV), e.g., an AAV comprising a polypeptide of the genetic modification system or exogenous polypeptide as a fusion, with or without a linker, to or with an AAV capsid protein such as VP1, VP2, and/or VP3. More in particular, modifying the knowledge in the art, e.g., Rybniker et al., “Incorporation of Antigens into Viral Capsids Augments Immunogenicity of Adeno-Associated Virus Vector-Based Vaccines,” J Virol. December 2012; 86(24): 13800-13804, Lux K, et al. 2005. Green fluorescent protein-tagged adeno-associated virus particles allow the study of cytosolic and nuclear trafficking. J. Virol. 79:11776-11787, Munch R C, et al. 2012. “Displaying high-affinity ligands on adeno-associated viral vectors enables tumor cell-specific and safe gene transfer.” Mol. Ther. [Epub ahead of print.] doi:10.1038/mt.2012.186 and Warrington K H, Jr, et al. 2004. Adeno-associated virus type 2 VP2 capsid protein is nonessential and can tolerate large peptide insertions at its N terminus. J. Virol. 78:6595-6609, each incorporated herein by reference, one can obtain a modified AAV capsid as described herein. It will be understood by those skilled in the art that the modifications described herein if inserted into the AAV cap gene may result in modifications in the VP1, VP2 and/or VP3 capsid subunits. Alternatively, the capsid subunits can be expressed independently to achieve modification in only one or two of the capsid subunits (VP1, VP2, VP3, VP1+VP2, VP1+VP3, or VP2+VP3). One can modify the cap gene to have expressed at a desired location a non-capsid protein advantageously a large payload protein, such as a Cas or other large exogenous protein. Likewise, these can be fusions, with the protein, e.g., large payload protein such as a Cas or other exogenous protein fused in a manner analogous to prior art fusions. See, e.g., US Patent Publication 20090215879; Nance et al., “Perspective on Adeno-Associated Virus Capsid Modification for Duchenne Muscular Dystrophy Gene Therapy,” Hum Gene Ther. 26(12):786-800 (2015), which can be modified and adapted for use to deliver and/or express a polynucleotide or system of the present description. The skilled person, from this disclosure and the knowledge in the art can make and use modified AAV or AAV capsid as with other aspects of the present disclosure, and through this description herein one knows now that large payload proteins can be fused to the AAV capsid. Accordingly, the approaches described herein are also applicable to a virus in the genus Dependoparvovirus or in the family Parvoviridae, for instance, AAV, or a virus of Amdoparvovirus, e.g., Carnivore amdoparvovirus 1, a virus of Aveparvovirus, e.g., Galliform aveparvovirus 1, a virus of Bocaparvovirus, e.g., Ungulate bocaparvovirus 1, a virus of Copiparvovirus, e.g., Ungulate copiparvovirus 1, a virus of Dependoparvovirus, e.g., Adeno-associated dependoparvovirus A, a virus of Erythroparvovirus, e.g., Primate erythroparvovirus 1, a virus of Protoparvovirus, e.g., Rodent protoparvovirus 1, a virus of Tetraparvovirus, e.g., Primate tetraparvovirus 1.

### Herpes Simplex Viral Vectors

In some embodiments, the vector is a Herpes Simplex Viral (HSV)-based vector or system thereof. HSV systems can include the disabled infections single copy (DISC) viruses, which are composed of a glycoprotein H defective mutant HSV genome. When the defective HSV is propagated in complementing cells, virus particles can be generated that are capable of infecting subsequent cells permanently replicating their own genome but are not capable of producing more infectious particles. See e.g., 2009. Trobridge. Exp. Opin. Biol. Ther. 9:1427-1436, whose techniques and vectors described therein can be modified and adapted for use in the CRISPR-Cas system of the present invention. In some embodiments where an HSV vector or system thereof is utilized, the host cell can be a complementing cell. In some embodiments, HSV vector or system thereof can be capable of producing virus particles capable of delivering a polynucleotide cargo of up to 150 kb. Thus, in some embodiments the cargo polynucleotide(s) included in the HSV-based viral vector or system thereof can sum from about 0.001 to about 150 kb. HSV-based vectors and systems thereof have been successfully used in several contexts including various models of neurologic disorders. See e.g., Cockrell et al. 2007. Mol. Biotechnol. 36:184-204; Kafri T. 2004. Mol. Biol. 246:367-390; Balaggan and Ali. 2012. Gene Ther. 19:145-153; Wong et al. 2006. Hum. Gen. Ther. 2002. 17:1-9; Azzouz et al. J. Neruosci. 22L10302-10312; and Betchen and Kaplitt. 2003. Curr. Opin. Neurol. 16:487-493, whose techniques and vectors described therein can be modified and adapted for use with the present disclosure.

### Poxvirus Vectors

In some embodiments, the vector can be a poxvirus vector or system thereof. In some embodiments, the poxvirus vector can result in cytoplasmic expression of one or more cargo polynucleotides of the present disclosure. In some embodiments the capacity of a poxvirus vector or system thereof can be about 25 kb or more. In some embodiments, a poxvirus vector or system thereof can include one or more cargo polynucleotides described herein.

### Nodaviral Vectors

In some embodiments, the vector is a nodaviral vector. Exemplary nodaviral vectors include, without limitation, any of those described in e.g., U.S. Pat. No. 6,514,757; Price et al., 2005. J. Virol. 79:495-502; Jariyapong, P. 2015. Artificial Cells, Nanomed, Biotec. 43(5) https://doi.org/10.3109/21691401.2014.889702; Tang et al., 2002, J. Virol. 6370-6375; WO/1999/029723; and International Application No.: PCT/US2022/035858, which may be modified and adapted to be used with the present disclosure.

### Baculoviral Vectors

In some embodiments, the vector is a baculoviral vector. Exemplary baculoviral vectors include, without limitation, any of those described in e.g., Kost et al., Nature Biotech. 23, p. 567-575 (2005); Felberbaum, R. Biotechnol J. 2015 May; 10(5): 702-714; Schaly et. al., 2021. Biologics: Targets and Therapy. 2021(15): 115-132. https://doi.org/10.2147/BTT.S292692; Lemaitre et al., BMC Biotechnology volume 19, Article number: 20 (2019); Matsuura et al., 1987. J General Virology. 68(5), https://doi.org/10.1099/0022-1317-68-5-1233; and Gomex-Sebastian et al., PLoSONE. 2014. https://doi.org/10.1371/journal.pone.0096562, which may be modified and adapted to be used with the present disclosure.

Virus Particle Production from Viral Vectors

### Retroviral Production

In some embodiments, one or more viral vectors and/or system thereof can be delivered to a suitable cell line for production of virus particles containing the polynucleotide or other payload to be delivered to a host cell. Suitable host cells for virus production from viral vectors and systems thereof described herein are known in the art and are commercially available. For example, suitable host cells include HEK 293 cells and its variants (HEK 293T and HEK 293TN cells). In some embodiments, the suitable host cell for virus production from viral vectors and systems thereof described herein can stably express one or more genes involved in packaging (e.g., pol, gag, and/or VSV-G) and/or other supporting genes.

In some embodiments, after delivery of one or more viral vectors to the suitable host cells for or virus production from viral vectors and systems thereof, the cells are incubated for an appropriate length of time to allow for viral gene expression from the vectors, packaging of the polynucleotide to be delivered (e.g., a genetic modifying system polynucleotide or other polynucleotide of the present disclosure), and virus particle assembly, and secretion of mature virus particles into the culture media. Various other methods and techniques are generally known to those of ordinary skill in the art.

Mature virus particles can be collected from the culture media by a suitable method. In some embodiments, this can involve centrifugation to concentrate the virus. The titer of the composition containing the collected virus particles can be obtained using a suitable method. Such methods can include transducing a suitable cell line (e.g., NIH 3T3 cells) and determining transduction efficiency, infectivity in that cell line by a suitable method. Suitable methods include PCR-based methods, flow cytometry, and antibiotic selection-based methods. Various other methods and techniques are generally known to those of ordinary skill in the art. The concentration of virus particle can be adjusted as needed. In some embodiments, the resulting composition containing virus particles can contain 1×10¹-1×10²⁰ or more particles/mL.

Lentiviruses may be prepared from any lentiviral vector or vector system described herein. In one example embodiment, after cloning a polynucleotide to be delivered into a suitable lentiviral vector (which contains a lentiviral transfer plasmid backbone), HEK293FT at low passage (p=5) can be seeded in a T-75 flask to 50% confluence the day before transfection in DMEM with 10% fetal bovine serum and without antibiotics. After 20 hours, the media can be changed to OptiMEM (serum-free) media and transfection of the lentiviral vectors can done 4 hours later. Cells can be transfected with 10 μg of lentiviral transfer plasmid (pCasES10) and the appropriate packaging plasmids (e.g., 5 μg of pMD2.G (VSV-g pseudotype), and 7.5 ug of psPAX2 (gag/pol/rev/tat)). Transfection can be carried out in 4 mL OptiMEM with a cationic lipid delivery agent (50 uL Lipofectamine 2000 and 100 ul Plus reagent). After 6 hours, the media can be changed to antibiotic-free DMEM with 10% fetal bovine serum. These methods can use serum during cell culture, but serum-free methods are preferred.

Following transfection and allowing the producing cells (also referred to as packaging cells) to package and produce virus particles with packaged cargo, the lentiviral particles can be purified. In an exemplary embodiment, virus-containing supernatants can be harvested after 48 hours. Collected virus-containing supernatants can first be cleared of debris and filtered through a 0.45 um low protein binding (PVDF) filter. They can then be spun in an ultracentrifuge for 2 hours at 24,000 rpm. The resulting virus-containing pellets can be resuspended in 50 ul of DMEM overnight at 4 degrees C. They can be then aliquoted and used immediately or immediately frozen at −80 degrees C for storage.

See also Merten et al., 2016. “Production of lentiviral vectors.” Mol. Ther. 3: 10617 for additional methods and techniques for lentiviral vector and particle production, which can be adapted for use with the present disclosure.

### AAV Particle Production

General principles of rAAV production are reviewed in, for example, Carter, 1992, Current Opinions in Biotechnology, 1533-539; and Muzyczka, 1992, Curr. Topics in Microbial. and Immunol., 158:97-129). Various approaches are described in Ratschin et al., Mol. Cell. Biol. 4:2072 (1984); Hermonat et al., Proc. Natl. Acad. Sci. USA, 81:6466 (1984); Tratschin et al., Mol. Cell. Biol. 5:3251 (1985); McLaughlin et al., J. Virol., 62:1963 (1988); and Lebkowski et al., 1988 Mol. Cell. Biol., 7:349 (1988). Samulski et al. (1989, J. Virol., 63:3822-3828); U.S. Pat. No. 5,173,414; WO 95/13365 and corresponding U.S. Pat. No. 5,658,776; WO 95/13392; WO 96/17947; PCT/US98/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin et al. (1995) Vaccine 13:1244-1250; Paul et al. (1993) Human Gene Therapy 4:609-615; Clark et al. (1996) Gene Therapy 3:1124-1132; U.S. Pat. Nos. 5,786,211; 5,871,982; and 6,258,595.

In general, there are two main strategies for producing AAV particles from AAV vectors and systems thereof, such as those described herein, which depend on how the adenovirus helper factors are provided (helper v. helper free). In some embodiments, a method of producing AAV particles from AAV vectors and systems thereof can include adenovirus infection into cell lines that stably harbor AAV replication and capsid encoding polynucleotides along with AAV vector containing the cargo polynucleotide to be packaged and delivered by the resulting AAV particle (e.g., the genetic modifying system polynucleotide(s)). In some embodiments, a method of producing AAV particles from AAV vectors and systems thereof can be a “helper free” method, which includes co-transfection of an appropriate producing cell line with three vectors (e.g., plasmid vectors): (1) an AAV vector that contains a cargo polynucleotide (e.g., the CRISPR-Cas system polynucleotide(s)) between 2 ITRs; (2) a vector that carries the AAV Rep-Cap encoding polynucleotides; and (helper polynucleotides). One of skill in the art will appreciate various methods and variations thereof that are both helper and -helper free and as well as the different advantages of each system. See also Kimur et al., 2019. Sci. Rep. 6:13601; Shin et al., Meth. Mol Biol. 2012. 798:267-284; Negrini et al., 2020. Curr. Prot. Neurosci. 93:e103; Dobrowsky et al., 2021. Curr. Op. Biomed. Eng. 20:100353 for additional methods and techniques for AAV vector and particle production, which can be adapted for use with the present disclosure.

### Non-Viral Vectors

In some embodiments, the vector is a non-viral vector or vector system. In some embodiments, the nudiviral nucleic acid is and/or engineered polynucleotide of the present disclosure is incorporated in a non-viral vector. In some embodiment a non-viral vector contains a nudiviral nucleic acid and/or engineered polynucleotide of the present disclosure. The term of art “Non-viral vector” and as used herein in this context refers to molecules and/or compositions that are vectors but that are not based on one or more component of a virus or virus genome (excluding any nucleotide to be delivered and/or expressed by the non-viral vector) that can be capable of incorporating cargo polynucleotide(s) and delivering said cargo polynucleotide(s) to a cell and/or expressing the polynucleotide in the cell. It will be appreciated that this does not exclude vectors containing a polynucleotide designed to target a virus-based polynucleotide that is to be delivered. For example, if a gRNA to be delivered is directed against a virus component and it is inserted or otherwise coupled to an otherwise non-viral vector or carrier, this would not make said vector a “viral vector”. Non-viral vectors can include, without limitation, naked polynucleotides and polynucleotide (non-viral) based vector and vector systems.

### Naked Polynucleotides

In some embodiments, one or more polynucleotides of the present disclosure described elsewhere herein can be included in a naked polynucleotide. The term of art “naked polynucleotide” as used herein refers to polynucleotides that are not associated with another molecule (e.g., proteins, lipids, and/or other molecules) that can often help protect it from environmental factors and/or degradation. As used herein, associated with includes, but is not limited to, linked to, adhered to, adsorbed to, enclosed in, enclosed in or within, mixed with, and the like. Naked polynucleotides that include one or more of the cargo polynucleotides described herein can be delivered directly to a host cell and optionally expressed therein. The naked polynucleotides can have any suitable two- and three-dimensional configurations. By way of non-limiting examples, naked polynucleotides can be single-stranded molecules, double stranded molecules, circular molecules (e.g., plasmids and artificial chromosomes), molecules that contain portions that are single stranded and portions that are double stranded (e.g., ribozymes), and the like. In some embodiments, the naked polynucleotide contains only the cargo polynucleotide(s). In some embodiments, the naked polynucleotide can contain other nucleic acids and/or polynucleotides in addition to the cargo polynucleotide(s). The naked polynucleotides can include one or more elements of a transposon system. Transposons and system thereof are described in greater detail elsewhere herein.

### Non-Viral Polynucleotide Vectors

In some embodiments, one or more of the polynucleotides of the present disclosure can be included in a non-viral polynucleotide vector. Suitable non-viral polynucleotide vectors include, but are not limited to, transposon vectors and vector systems, plasmids, bacterial artificial chromosomes, yeast artificial chromosomes, AR(antibiotic resistance)-free plasmids and miniplasmids, circular covalently closed vectors (e.g. minicircles, minivectors, miniknots,), linear covalently closed vectors (“dumbbell shaped”), MIDGE (minimalistic immunologically defined gene expression) vectors, MiLV (micro-linear vector) vectors, Ministrings, mini-intronic plasmids, PSK systems (post-segregationally killing systems), ORT (operator repressor titration) plasmids, and the like. See e.g., Hardee et al. 2017. Genes. 8(2):65.

In some embodiments, the non-viral polynucleotide vector can have a conditional origin of replication. In some embodiments, the non-viral polynucleotide vector can be an ORT plasmid. In some embodiments, the non-viral polynucleotide vector can have a minimalistic immunologically defined gene expression. In some embodiments, the non-viral polynucleotide vector can have one or more post-segregationally killing system genes. In some embodiments, the non-viral polynucleotide vector is AR-free. In some embodiments, the non-viral polynucleotide vector is a minivector. In some embodiments, the non-viral polynucleotide vector includes a nuclear localization signal. In some embodiments, the non-viral polynucleotide vector can include one or more CpG motifs. In some embodiments, the non-viral polynucleotide vectors can include one or more scaffold/matrix attachment regions (S/MARs). See e.g., Mirkovitch et al. 1984. Cell. 39:223-232, Wong et al. 2015. Adv. Genet. 89:113-152, whose techniques and vectors can be adapted for use in the present invention. S/MARs are AT-rich sequences that play a role in the spatial organization of chromosomes through DNA loop base attachment to the nuclear matrix. S/MARs are often found close to regulatory elements such as promoters, enhancers, and origins of DNA replication. Inclusion of one or S/MARs can facilitate a once-per-cell-cycle replication to maintain the non-viral polynucleotide vector as an episome in daughter cells. In certain embodiments, the S/MAR sequence is located downstream of an actively transcribed polynucleotide (e.g., one or more cargo polynucleotides) included in the non-viral polynucleotide vector. In some embodiments, the S/MAR can be a S/MAR from the beta-interferon gene cluster. See e.g., Verghese et al. 2014. Nucleic Acid Res. 42:e53; Xu et al. 2016. Sci. China Life Sci. 59:1024-1033; Jin et al. 2016. 8:702-711; Koirala et al. 2014. Adv. Exp. Med. Biol. 801:703-709; and Nehlsen et al. 2006. Gene Ther. Mol. Biol. 10:233-244, whose techniques and vectors can be adapted for use in the present invention.

### Transposons

In some embodiments, the non-viral vector is a transposon vector or system thereof. As used herein, “transposon” (also referred to as transposable element) refers to a polynucleotide sequence that is capable of moving form location in a genome to another. There are several classes of transposons. Transposons include retrotransposons and DNA transposons. Retrotransposons require the transcription of the polynucleotide that is moved (or transposed) in order to transpose the polynucleotide to a new genome or polynucleotide. DNA transposons are those that do not require reverse transcription of the polynucleotide that is moved (or transposed) in order to transpose the polynucleotide to a new genome or polynucleotide. In some embodiments, the non-viral polynucleotide vector can be a retrotransposon vector. In some embodiments, the retrotransposon vector includes long terminal repeats. In some embodiments, the retrotransposon vector does not include long terminal repeats. In some embodiments, the non-viral polynucleotide vector can be a DNA transposon vector. DNA transposon vectors can include a polynucleotide sequence encoding a transposase. In some embodiments, the transposon vector is configured as a non-autonomous transposon vector, meaning that the transposition does not occur spontaneously on its own. In some of these embodiments, the transposon vector lacks one or more polynucleotide sequences encoding proteins required for transposition. In some embodiments, the non-autonomous transposon vectors lack one or more Ac elements.

In some embodiments a non-viral polynucleotide transposon vector system can include a first polynucleotide vector that contains the cargo polynucleotide(s) of the present invention flanked on the 5′ and 3′ ends by transposon terminal inverted repeats (TIRs) and a second polynucleotide vector that includes a polynucleotide capable of encoding a transposase coupled to a promoter to drive expression of the transposase. When both are expressed in the same cell the transposase can be expressed from the second vector and can transpose the material between the TIRs on the first vector (e.g., the cargo polynucleotide(s) of the present invention) and integrate it into one or more positions in the host cell's genome. In some embodiments the transposon vector or system thereof can be configured as a gene trap. In some embodiments, the TTRs can be configured to flank a strong splice acceptor site followed by a reporter and/or other gene (e.g., one or more of the cargo polynucleotide(s) of the present invention) and a strong poly A tail. When transposition occurs while using this vector or system thereof, the transposon can insert into an intron of a gene and the inserted reporter or other gene can provoke a mis-splicing process and as a result it in activates the trapped gene.

Any suitable transposon system can be used. Suitable transposon and systems thereof can include without limitation Sleeping Beauty transposon system (Tc1/mariner superfamily) (see e.g., Ivics et al. 1997. Cell. 91(4): 501-510), piggyBac (piggyBac superfamily) (see e.g., Li et al. 2013 110(25): E2279-E2287 and Yusa et al. 2011. PNAS. 108(4): 1531-1536), To12 (superfamily hAT), Frog Prince (Tc1/mariner superfamily) (see e.g., Miskey et al. 2003 Nucleic Acid Res. 31(23):6873-6881) and variants thereof.

### Delivery of the Polynucleotides and Vectors

The engineered polynucleotides and/or vectors can be delivered to a cell or cell population using any suitable delivery composition, system or technique. Non-limiting exemplary techniques and compositions are discussed in greater detail below.

### Physical Delivery

In some embodiments, the engineered polynucleotides and/or vectors, etc. of the present disclosure may be introduced to cells by physical delivery methods. Examples of physical methods include microinjection, electroporation, ballistic methods, and hydrodynamic delivery. Both nucleic acid and proteins may be delivered using such methods.

### Microinjection

In some embodiments, delivery of the engineered polynucleotides and/or vectors, etc. of the present disclosure is via microinjection. Microinjection of the engineered polynucleotides and/or vectors directly to cells can achieve high efficiency, e.g., above 90% or about 100%. In some embodiments, microinjection may be performed using a microscope and a needle (e.g., with 0.5-5.0 m in diameter) to pierce a cell membrane and deliver the cargo directly to a target site within the cell. Microinjection may be used for in vitro and ex vivo delivery.

Microinjection may be used to generate genetically modified animals. For example, gene modification systems or components thereof may be injected into zygotes, blastomeres, blastocysts, embryonic stem cells, pluripotent stem cells, induced pluripotent stem cells, primordial germ cells, primordial germ cell like-cells, and/or the like to allow for gene medication, such as germline modification.

### Electroporation

In some embodiments, the engineered polynucleotides and/or vectors or other delivery vehicles containing the same described herein may be delivered by electroporation. Electroporation may use pulsed high-voltage electrical currents to transiently open nanometer-sized pores within the cellular membrane of cells suspended in buffer, allowing for components with hydrodynamic diameters of tens of nanometers to flow into the cell. In some cases, electroporation may be used on various cell types and efficiently transfer cargo into cells. Electroporation may be used for in vitro and ex vivo delivery.

Electroporation may also be used to deliver the cargo to into the nuclei of mammalian cells by applying specific voltage and reagents, e.g., by nucleofection. Such approaches include those described in Wu Y, et al. (2015). Cell Res 25:67-79; Ye L, et al. (2014). Proc Natl Acad Sci USA 111:9591-6; Choi P S, Meyerson M. (2014). Nat Commun 5:3728; Wang J, Quake S R. (2014). Proc Natl Acad Sci 111:13157-62. Electroporation may also be used to deliver the cargo in vivo, e.g., with methods described in Zuckermann M, et al. (2015). Nat Commun 6:7391.

### Hydrodynamic Delivery

Hydrodynamic delivery may also be used for delivering the engineered polynucleotides and/or vectors, etc. of the present disclosure, e.g., for in vivo delivery. In some examples, hydrodynamic delivery may be performed by rapidly pushing a large volume (8-10% body weight) solution containing the gene modification system into the bloodstream of a subject (e.g., a bovine). As blood is incompressible, the large bolus of liquid may result in an increase in hydrodynamic pressure that temporarily enhances permeability into endothelial and parenchymal cells, allowing for cargo not normally capable of crossing a cellular membrane to pass into cells. This approach may be used for delivering naked DNA plasmids and proteins. The delivered genetic modification system or components may be enriched in ovaries and/or testis.

### Transfection

The engineered polynucleotides and/or vectors, etc. of the present invention, may be introduced to cells by transfection methods for introducing nucleic acids into cells. Examples of transfection methods include calcium phosphate-mediated transfection, cationic transfection, liposome transfection, dendrimer transfection, heat shock transfection, magnetofection, lipofection, impalefection, optical transfection, proprietary agent-enhanced uptake of nucleic acid. Nucleic acids and vectors and vector systems that can encode a genetic modifying system and/or components thereof are described in greater detail else wherein herein.

### Transduction

The engineered polynucleotides and/or vectors, etc. of the present disclosure can be introduced to cells by transduction by a viral, pseudoviral, and/or virus like particle. Methods of packaging the genetic modifying systems and/or components thereof in viral particles can be accomplished using any suitable viral vector or vector systems. Such viral vector and vector systems are described in greater detail elsewhere herein. As used in this context herein “transduction” refers to the process by which foreign nucleic acids and/or proteins are introduced to a cell (prokaryote or eukaryote) by a viral, pseudoviral, and/or virus like particle. After packaging in a viral, pseudoviral, and/or virus like particle. the viral particles can be exposed to cells (e.g., in vitro, ex vivo, or in vivo) where the viral, pseudoviral, and/or virus like particle infects the cell and delivers the cargo to the cell via transduction. Viral, pseudoviral, and/or virus like particles can be optionally concentrated prior to exposure to target cells. In some embodiments, the virus titer of a composition containing viral and/or pseudoviral particles can be obtained and a specific titer be used to transduce cells. Viral vectors and systems and generation of viral (or pseudoviral, and/or virus like particle) delivery particles is described in greater detail elsewhere herein. Viral transduction has been used to deliver exogenous nucleic acid constructs to bovine cells. See e.g., Hoffmann et al., Biology of Reproduction, Volume 71, Issue 2, 1 Aug. 2004, Pages 405-409, doi.org/10.1095/biolreprod.104.028472; Yu et al., (2014) Expression of Intracellular Interferon-Alpha Confers Antiviral Properties in Transfected Bovine Fetal Fibroblasts and Does Not Affect the Full Development of SCNT Embryos. PLoS ONE 9(7): e94444, doi.org/10.1371/journal.pone.0094444; and Wu et al., Scientific Reports volume 6, Article number: 28343 (2016), which are incorporated by reference as if expressed in their entireties herein and can be adapted for use with the present disclosure.

### Biolistics

The engineered polynucleotides and/or vectors, etc. of the present disclosure can be introduced to cells using a biolistic method or technique. The term of art “biolistic”, as used herein, refers to the delivery of nucleic acids to cells by high-speed particle bombardment. In some embodiments, an engineered polynucleotide or vector of the present disclosure can be introduced can be attached, associated with, or otherwise coupled to particles, which than can be delivered to the cell via a gene-gun (see e.g., Liang et al. 2018. Nat. Protocol. 13:413-430; Svitashev et al. 2016. Nat. Comm. 7:13274; Ortega-Escalante et al., 2019. Plant. J. 97:661-672). In some embodiments, the particles can be gold, tungsten, palladium, rhodium, platinum, or iridium particles.

In particular embodiments, an engineered polynucleotide or vector of the present disclosure can be introduced into the plant cell using techniques such as but not limited to electroporation, microinjection, aerosol beam injection of plant cell protoplasts, or the an engineered polynucleotide or vector of the present disclosure can be introduced can be introduced directly to plant tissue using biolistic methods, such as DNA particle bombardment (see also Fu et al., Transgenic Res. 2000 February; 9(1):11-9). The basis of particle bombardment is the acceleration of particles coated with gene/s of interest toward cells, resulting in the penetration of the protoplasm by the particles and typically stable integration into the genome. (see e.g., Klein et al, Nature (1987), Klein et ah, Bio/Technology (1992), Casas et ah, Proc. Natl. Acad. Sci. USA (1993).).

### Implantable Devices

In some embodiments, the delivery system can include an implantable device that incorporates or is coated with a genetic modifying systems and/or components thereof described herein. Various implantable devices are described in the art, and include any device, graft, or other composition that can be implanted into a subject, such as a bovine.

### Delivery Vehicles

The engineered polynucleotides and/or vectors, etc. of the present disclosure can be delivered to a cell using a suitable delivery vehicle. The delivery vehicles can deliver a cargo, such as a polynucleotide or polypeptide of the present disclosure into cells, tissues, organs, or organisms (e.g., animals or plants). The cargos may be packaged, carried, or otherwise associated with the delivery vehicles. The delivery vehicles may be selected based on the types of cargo to be delivered, and/or the delivery is in vitro and/or in vivo. Examples of delivery vehicles include vectors, viruses (e.g., virus particles, pseudoviral particles, or virus like particles), non-viral vehicles (e.g., exosomes, liposomes, etc.), and other delivery reagents described herein and those appreciated by one of ordinary skill in the art in view of the present disclosure. Some delivery vehicles are described elsewhere herein, such as vectors and virus or viral like particles. Additional exemplary delivery vehicles, such as non-vector delivery vehicles, are now described in greater detail. Examples of non-vector vehicles include lipid nanoparticles, cell-penetrating peptides (CPPs), DNA nanoclews, metal nanoparticles, streptolysin O, multifunctional envelope-type nanodevices (MENDs), lipid-coated mesoporous silica particles, and other inorganic nanoparticles.

In some embodiments the delivery vehicles described herein can have a greatest dimension or greatest average dimension (e.g., diameter or greatest average diameter) of less than 100 microns (μm). In some embodiments, the delivery vehicles have a greatest dimension or greatest average dimension of less than 10 μm. In some embodiments, the delivery vehicles may have a greatest dimension or greatest average dimension of less than 2000 nanometers (nm). In some embodiments, the delivery vehicles may have a greatest dimension or greatest average dimension of less than 1000 nanometers (nm). In some embodiments, the delivery vehicles may have a greatest dimension or greatest average dimension (e.g., diameter or average diameter) of less than 900 nm, less than 800 nm, less than 700 nm, less than 600 nm, less than 500 nm, less than 400 nm, less than 300 nm, less than 200 nm, less than 150 nm, or less than 100 nm, less than 50 nm. In some embodiments, the delivery vehicles may have a greatest dimension or greatest average dimension ranging between 25 nm and 200 nm.

### Particles

In some embodiments, the delivery vehicles may be or comprise particles. For example, the delivery vehicle may be or comprise nanoparticles (e.g., particles with a greatest dimension or greatest average dimension (e.g., diameter or greatest average diameter) no greater than 1000 nm. The particles may be provided in different forms, e.g., as solid particles (e.g., metal such as silver, gold, iron, titanium), non-metal, lipid-based solids, polymers), suspensions of particles, or combinations thereof. Metal, dielectric, and semiconductor particles may be prepared, as well as hybrid structures (e.g., core-shell particles).

Nanoparticles may also be used to deliver the compositions and systems to cells, as described in US20130185823, WO2008042156, and WO2015089419. In general, a “nanoparticle” refers to any particle having a diameter of less than 1000 nm. In certain embodiments, nanoparticles of the invention have a greatest dimension or greatest average dimension (e.g., diameter or average diameter) of 500 nm or less. In other embodiments, nanoparticles of the invention have a greatest dimension or greatest average dimension ranging between 25 nm and 200 nm. In other embodiments, nanoparticles of the invention have a greatest dimension or greatest average dimension of 100 nm or less. In other embodiments, nanoparticles of the invention have a greatest dimension or greatest average dimensions ranging between 35 nm and 60 nm. It will be appreciated that reference made herein to particles or nanoparticles can be interchangeable, where appropriate. Nanoparticles made of semiconducting material may also be labeled quantum dots if they are small enough (typically sub 10 nm) that quantization of electronic energy levels occurs. Such nanoscale particles are used in biomedical applications as drug carriers or imaging agents and may be adapted for similar purposes in the present invention. Semi-solid and soft nanoparticles have been manufactured and are within the scope of the present invention. Nanoparticles with one half hydrophilic and the other half hydrophobic are termed Janus particles and are particularly effective for stabilizing emulsions. They can self-assemble at water/oil interfaces and act as solid surfactants.

Particle characterization (including e.g., characterizing morphology, dimension, etc.) is done using a variety of different techniques. Common techniques are electron microscopy (TEM, SEM), atomic force microscopy (AFM), dynamic light scattering (DLS), X-ray photoelectron spectroscopy (XPS), powder X-ray diffraction (XRD), Fourier transform infrared spectroscopy (FTIR), matrix-assisted laser desorption/ionization time-of-flight mass spectrometry (MALDI-TOF), ultraviolet-visible spectroscopy, dual polarization interferometry and nuclear magnetic resonance (NMR). Characterization (dimension measurements) may be made as to native particles (i.e., preloading) or after loading of the cargo (e.g., one or more components of a genetic modifying system (e.g., a CRISPR-Cas system or component(s) thereof) and can include additional carriers and/or excipients) to provide particles of an optimal size for delivery for any in vitro, ex vivo and/or in vivo application of the present disclosure. In some embodiments, particle dimension (e.g., diameter) characterization is based on measurements using dynamic laser scattering (DLS). See also e.g., U.S. Pat. Nos. 8,709,843; 6,007,845; 5,855,913; 5,985,309; 5,543,158; and Dahlman et al. Nature Nanotechnology (2014), doi:10.1038/nnano.2014.84, describes particles, methods of making and using them, and measurements thereof which can be adapted for use with the present disclosure.

### Lipid Particles

The delivery vehicles can include or be composed of lipid particles, e.g., lipid nanoparticles (LNPs) and liposomes. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner, International Patent Publication Nos. WO 91/17424 and WO 91/16024. The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

### Lipid Nanoparticles (LNPs)

LNPs may encapsulate nucleic acids within cationic lipid particles (e.g., liposomes), and may be delivered to cells with relative ease. In some examples, lipid nanoparticles do not contain any viral components, which helps minimize safety and immunogenicity concerns. Lipid particles may be used for in vitro, ex vivo, and in vivo deliveries. Lipid particles may be used for various scales of cell populations.

In some examples. LNPs may be used for delivering DNA molecules (e.g., those comprising coding sequences of a cargo polypeptide) and/or RNA molecules (e.g., mRNA of encoding a cargo polypeptide and/or other RNA cargos such as gRNAs). In certain cases, LNPs may be use for delivering RNP complexes of e.g., Cas/gRNA.

Components in LNPs may comprise cationic lipids 1,2-dilineoyl-3-dimethylammonium-propane (DLinDAP), 1,2-dilinoleyloxy-3-N,N-dimethylaminopropane (DLinDMA), 1,2-dilinoleyloxyketo-N,N-dimethyl-3-aminopropane (DLinK-DMA), 1,2-dilinoleyl-4-(2-dimethylaminoethyl)-[1,3]-dioxolane (DLinKC2-DMA), (3-o-[2″-(methoxypolyethyleneglycol 2000) succinoyl]-1,2-dimyristoyl-sn-glycol (PEG-S-DMG), R-3-[(ro-methoxy-poly(ethylene glycol)2000) carbamoyl]-1,2-dimyristyloxlpropyl-3-amine (PEG-C-DOMG, and any combination thereof. Preparation of LNPs and encapsulation may be adapted from Rosin et al, Molecular Therapy, vol. 19, no. 12, pages 1286-2200, December 2011).

In some embodiments, an LNP delivery vehicle can be used to deliver a virus particle containing cargo polypeptides or polynucleotides. In some embodiments, the virus particle(s) can be adsorbed to the lipid particle, such as through electrostatic interactions, and/or can be attached to the liposomes via a linker.

In some embodiments, the LNP contains a nucleic acid, wherein the charge ratio of nucleic acid backbone phosphates to cationic lipid nitrogen atoms is about 1:1.5-7 or about 1:4.

In some embodiments, the LNP also includes a shielding compound, which is removable from the lipid composition under in vivo conditions. In some embodiments, the shielding compound is a biologically inert compound. In some embodiments, the shielding compound does not carry any charge on its surface or on the molecule as such. In some embodiments, the shielding compounds are polyethylenglycoles (PEGs), hydroxyethylglucose (HEG) based polymers, polyhydroxyethyl starch (polyHES) and polypropylene. In some embodiments, the PEG, HEG, polyHES, and a polypropylene weight between about 500 to 10,000 Da or between about 2000 to 5000 Da. In some embodiments, the shielding compound is PEG2000 or PEG5000.

In some embodiments, the LNP can include one or more helper lipids. In some embodiments, the helper lipid can be a phosphor lipid or a steroid. In some embodiments, the helper lipid is between about 20 mol % to 80 mol % of the total lipid content of the composition. In some embodiments, the helper lipid component is between about 35 mol % to 65 mol % of the total lipid content of the LNP. In some embodiments, the LNP includes lipids at 50 mol % and the helper lipid at 50 mol % of the total lipid content of the LNP.

Other non-limiting, exemplary LNP delivery vehicles are described in U.S. Patent Publication Nos. US 20160174546, US 20140301951, US 20150105538, US 20150250725, Wang et al., J. Control Release, 2017 Jan. 31. pii: S0168-3659(17)30038-X. doi: 10.1016/j.jconrel.2017.01.037.; Altmoglu et al., Biomater Sci., 4(12):1773-80, Nov. 15, 2016; Wang et al., PNAS, 113(11):2868-73 Mar. 15, 2016; Wang et al., PloS One, 10(11): e0141860. doi: 10.1371/journal.pone.0141860. eCollection 2015, Nov. 3, 2015; Takeda et al., Neural Regen Res. 10(5):689-90, May 2015; Wang et al., Adv. Healthc Mater., 3(9):1398-403, September 2014; and Wang et al., Agnew Chem Int Ed Engl., 53(11):2893-8, Mar. 10, 2014; James E. Dahlman and Carmen Barnes et al. Nature Nanotechnology (2014) published online 11 May 2014, doi:10.1038/nnano.2014.84; Coelho et al., N Engl J Med 2013; 369:819-29; Aleku et al., Cancer Res., 68(23): 9788-98 (Dec. 1, 2008), Strumberg et al., Int. J. C/in. Pharmacol. Ther., 50(1): 76-8 (January 2012), Schultheis et al., J. C/in. Oncol., 32(36): 4141-48 (Dec. 20, 2014), and Fehring et al., Mol. Ther., 22(4): 811-20 (Apr. 22, 2014); Novobrantseva, Molecular Therapy-Nucleic Acids (2012) 1, e4; doi:10.1038/mtna.2011.3; WO2012135025; US 20140348900; US 20140328759; US 20140308304; WO 2005/105152; WO 2006/069782; WO 2007/121947; US 2015/082080; US 20120251618; 7,982,027; 7,799,565; 8,058,069; 8,283,333; 7,901,708; 7,745,651; 7,803,397; 8,101,741; 8,188,263; 7,915,399; 8,236,943 and 7,838,658 and European Pat. Nos 1766035; 1519714; 1781593 and 1664316.

### Liposomes

In some embodiments, a lipid particle may be liposome. Liposomes are spherical vesicle structures composed of a uni- or multilamellar lipid bilayer surrounding internal aqueous compartments and a relatively impermeable outer lipophilic phospholipid bilayer. In some embodiments, liposomes are biocompatible, nontoxic, can deliver both hydrophilic and lipophilic drug molecules, protect their cargo from degradation by plasma enzymes, and transport their load across biological membranes and the blood brain barrier (BBB).

Liposomes can be made from several different types of lipids, e.g., phospholipids. A liposome may comprise natural phospholipids and lipids such as 1,2-distearoryl-sn-glycero-3-phosphatidyl choline (DSPC), sphingomyelin, egg phosphatidylcholines, monosialoganglioside, or any combination thereof.

Several other additives may be added to liposomes in order to modify their structure and properties. For instance, liposomes may further comprise cholesterol, sphingomyelin, and/or 1,2-dioleoyl-sn-glycero-3-phosphoethanolamine (DOPE), e.g., to increase stability and/or to prevent the leakage of the liposomal inner cargo.

In some embodiments, a liposome delivery vehicle can be used to deliver a virus particle containing cargo polypeptide(s) and/or polynucleotide(s). In some embodiments, the virus particle(s) can be adsorbed to the liposome, such as through electrostatic interactions, and/or can be attached to the liposomes via a linker.

In some embodiments, the liposome can be a Trojan Horse liposome (also known in the art as Molecular Trojan Horses), see e.g., cshprotocols.cshlp.org/content/2010/4/pdb.prot5407.long, the teachings of which can be applied and/or adapted to generated and/or deliver the genetic modifying systems and/or other cargo polypeptides or polynucleotides described herein.

Other non-limiting, exemplary liposomes can be those as set forth in Wang et al., ACS Synthetic Biology, 1, 403-07 (2012); Wang et al., PNAS, 113(11) 2868-2873 (2016); Spuch and Navarro, Journal of Drug Delivery, vol. 2011, Article ID 469679, 12 pages, 2011. doi:10.1155/2011/469679; WO 2008/042973; U.S. Pat. No. 8,071,082; WO 2014/186366; 20160257951; US 20160129120; US 20160244761; US 20120251618; WO 2013/093648; Lipofectin (a combination of DOTMA and DOPE), Lipofectase, LIPOFECTAMINE® (e.g., LIPOFECTAMINE® 2000, LIPOFECTAMINE® 3000, LIPOFECTAMINE® RNAiMAX, LIPOFECTAMINE® LTX), SAINT-RED (Synvolux Therapeutics, Groningen Netherlands), DOPE, Cytofectin (Gilead Sciences, Foster City, Calif.), and Eufectins (JBL, San Luis Obispo, Calif.).

### Stable Nucleic-Acid-Lipid Particles (SNALPs)

In some embodiments, the lipid particles contain or are composed entirely of stable nucleic acid lipid particles (SNALPs). SNALPs may comprise an ionizable lipid (DLinDMA) (e.g., cationic at low pH), a neutral helper lipid, cholesterol, a diffusible polyethylene glycol (PEG)-lipid, or any combination thereof. In some examples, SNALPs may comprise synthetic cholesterol, dipalmitoylphosphatidylcholine, 3-N-[(w-methoxy polyethylene glycol)2000)carbamoyl]-1,2-dimyrestyloxypropylamine, and cationic 1,2-dilinoleyloxy-3-N,Ndimethylaminopropane. In some examples, SNALPs may comprise synthetic cholesterol, 1,2-distearoyl-sn-glycero-3-phosphocholine, PEG-cDMA, and 1,2-dilinoleyloxy-3-(N;N-dimethyl)aminopropane (DLinDMAo).

Other non-limiting, exemplary SNALPs that can be used to deliver the cargos described herein can be any such SNALPs as described in Morrissey et al., Nature Biotechnology, Vol. 23, No. 8, August 2005, Zimmerman et al., Nature Letters, Vol. 441, 4 May 2006; Geisbert et al., Lancet 2010; 375: 1896-905; Judge, J. Clin. Invest. 119:661-673 (2009); and Semple et al., Nature Niotechnology, Volume 28 Number 2 Feb. 2010, pp. 172-177.

### Other Lipids

The lipid particles may also comprise one or more other types of lipids, e.g., cationic lipids, such as amino lipid 2,2-dilinoleyl-4-dimethylaminoethyl-[1,3]-dioxolane (DLin-KC2-DMA), DLin-KC2-DMA4, C12-200 and colipids disteroylphosphatidyl choline, cholesterol, and PEG-DMG.

In some embodiments, the delivery vehicle can be or include a lipidoid, such as any of those set forth in, for example, US 20110293703.

In some embodiments, the delivery vehicle can be or include an amino lipid, such as any of those set forth in, for example, Jayaraman, Angew. Chem. Int. Ed. 2012, 51, 8529-8533.

In some embodiments, the delivery vehicle can be or include a lipid envelope, such as any of those set forth in, for example, Korman et al., 2011. Nat. Biotech. 29:154-157.

### Lipoplexes Polyplexes

In some embodiments, the delivery vehicles contain or be composed entirely of lipoplexes and/or polyplexes. Lipoplexes may bind to negatively charged cell membrane and induce endocytosis into the cells. Examples of lipoplexes may be complexes comprising lipid(s) and non-lipid components. Examples of lipoplexes and polyplexes include FuGENE-6 reagent, a non-liposomal solution containing lipids and other components, zwitterionic amino lipids (ZALs), Ca2 [symbol] (e.g., forming DNA/Ca²⁺ microcomplexes), polyethenimine (PEI) (e.g., branched PEI), and poly(L-lysine) (PLL).

### Sugar-Based Particles

In some embodiments, the delivery vehicle can be a sugar-based particle. In some embodiments, the sugar-based particles can be or include GalNAc, such as any of those described in WO2014118272; US 20020150626; Nair, J K et al., 2014, Journal of the American Chemical Society 136 (49), 16958-16961; Ostergaard et al., Bioconjugate Chem., 2015, 26 (8), pp 1451-1455.

### Cell Penetrating Peptides

In some embodiments, the delivery vehicles contain or are composed entirely of cell penetrating peptides (CPPs). CPPs are short peptides that facilitate cellular uptake of various molecular cargo (e.g., from nanosized particles to small chemical molecules and large fragments of DNA).

CPPs may be of different sizes, amino acid sequences, and charges. In some examples, CPPs can translocate the plasma membrane and facilitate the delivery of various molecular cargoes to the cytoplasm or an organelle. CPPs may be introduced into cells via different mechanisms, e.g., direct penetration in the membrane, endocytosis-mediated entry, and translocation through the formation of a transitory structure.

CPPs may have an amino acid composition that either contains a high relative abundance of positively charged amino acids such as lysine or arginine or has sequences that contain an alternating pattern of polar/charged amino acids and non-polar, hydrophobic amino acids. These two types of structures are referred to as polycationic or amphipathic, respectively. A third class of CPPs are the hydrophobic peptides, containing only apolar residues, with low net charge or have hydrophobic amino acid groups that are crucial for cellular uptake. Another type of CPPs is the trans-activating transcriptional activator (Tat) from Human Immunodeficiency Virus 1 (HIV-1). Examples of CPPs include to Penetratin, Tat (48-60), Transportan, and (R-AhX-R4) (Ahx refers to aminohexanoyl), Kaposi fibroblast growth factor (FGF) signal peptide sequence, integrin 33 signal peptide sequence, polyarginine peptide Args sequence, Guanine rich-molecular transporters, and sweet arrow peptide. Examples of CPPs and related applications also include those described in U.S. Pat. No. 8,372,951.

CPPs can be used for in vitro and ex vivo work quite readily, and extensive optimization for each cargo and cell type is usually required. In some examples, CPPs may be covalently attached to the Cas protein directly, which is then complexed with the gRNA and delivered to cells. In some examples, separate delivery of CPP-Cas and CPP-gRNA to multiple cells may be performed. CPP may also be used to delivery RNPs.

CPPs may be used to deliver the compositions and systems to plants. In some examples, CPPs may be used to deliver the components to plant protoplasts, which are then regenerated to plant cells and further to plants.

### DNA Nanoclews

In some embodiments, the delivery vehicles contain or are composed entirely of DNA nanoclews. A DNA nanoclew refers to a sphere-like structure of DNA (e.g., with a shape of a ball of yarn). The nanoclew may be synthesized by rolling circle amplification with palindromic sequences that aide in the self-assembly of the structure. The sphere may then be loaded with a payload. An example of DNA nanoclew is described in Sun W et al, J Am Chem Soc. 2014 Oct. 22; 136(42):14722-5; and Sun W et al, Angew Chem Int Ed Engl. 2015 Oct. 5; 54(41):12029-33. DNA nanoclew may have a palindromic sequences to be partially complementary to the gRNA within the Cas:gRNA ribonucleoprotein complex. A DNA nanoclew may be coated, e.g., coated with PEI to induce endosomal escape.

### Metal Nanoparticles

in some embodiments, the delivery vehicles contain or are composed entirely of metal nanoparticles. In some embodiments, the delivery vehicles contain or are composed entirely of gold nanoparticles (also referred to AuNPs or colloidal gold). Gold nanoparticles may form complex with cargos, e.g., Cas:gRNA RNP. Gold nanoparticles may be coated, e.g., coated in a silicate and an endosomal disruptive polymer, PAsp(DET). Examples of gold nanoparticles include AuraSense Therapeutics' Spherical Nucleic Acid (SNA™) constructs, and those described in Mout R, et al. (2017). ACS Nano 11:2452-8; Lee K, et al. (2017). Nat Biomed Eng 1:889-901. Other metal nanoparticles can also be complexed with cargo(s). Such metal nanoparticles include, without limitation, tungsten, palladium, rhodium, platinum, and iridium particles. Other non-limiting, exemplary metal nanoparticles suitable for delivery vehicles are described in US 20100129793.

iTOP

In some embodiments, the delivery vehicles contain or are composed entirely of iTOP. iTOP refers to a combination of small molecules drives the highly efficient intracellular delivery of native proteins, independent of any transduction peptide. iTOP may be used for induced transduction by osmocytosis and propanebetaine, using NaCl-mediated hyperosmolality together with a transduction compound (propanebetaine) to trigger macropinocytotic uptake into cells of extracellular macromolecules. Examples of iTOP methods and reagents include those described in D'Astolfo D S, Pagliero R J, Pras A, et al. (2015). Cell 161:674-690.

### Polymer-Based Particles

In some embodiments, the delivery vehicles contain or are composed entirely of polymer-based particles (e.g., nanoparticles). In some embodiments, the polymer-based particles may mimic a viral mechanism of membrane fusion. The polymer-based particles may be a synthetic copy of Influenza virus machinery and form transfection complexes with various types of nucleic acids (siRNA, miRNA, plasmid DNA or shRNA, mRNA) that cells take up via the endocytosis pathway, a process that involves the formation of an acidic compartment. The low pH in late endosomes acts as a chemical switch that renders the particle surface hydrophobic and facilitates membrane crossing. Once in the cytosol, the particle releases its payload for cellular action. This Active Endosome Escape technology is safe and maximizes transfection efficiency as it is using a natural uptake pathway. In some embodiments, the polymer-based particles may comprise alkylated and carboxyalkylated branched polyethylenimine. In some examples, the polymer-based particles are VIROMER, e.g., VIROMER RNAi, VIROMER RED, VIROMER mRNA, VIROMER CRISPR. Example methods of delivering the systems and compositions herein include those described in Bawage S S et al., Synthetic mRNA expressed Cas13a mitigates RNA virus infections, www.biorxiv.org/content/10.1101/370460v1.full doi: doi.org/10.1101/370460, Viromer® RED, a powerful tool for transfection of keratinocytes. doi: 10.13140/RG.2.2.16993.61281, Viromer® Transfection—Factbook 2018: technology, product overview, users' data, doi:10.13140/RG.2.2.23912.16642. Other exemplary and non-limiting polymeric particles suitable for delivery vehicles are described in US 20170079916, US 20160367686, US 20110212179, US 20130302401, 6,007,845, 5,855,913, 5,985,309, 5,543,158, WO2012135025, US 20130252281, US 20130245107, US 20130244279; US 20050019923, 20080267903.

### Streptolvsin O (SLO)

The delivery vehicles can contain or be composed entirely of streptolysin 0 (SLO). SLO is a toxin produced by Group A streptococci that works by creating pores in mammalian cell membranes. SLO may act in a reversible manner, which allows for the delivery of proteins (e.g., up to 100 kDa) to the cytosol of cells without compromising overall viability. Examples of SLO include those described in Sierig G, et al. (2003). Infect Immun 71:446-55; Walev I, et al. (2001). Proc Natl Acad Sci USA 98:3185-90; Teng K W, et al. (2017). Elife 6:e25460.

### Multifunctional Envelope-Type Nanodevice (MEND)

The delivery vehicles can contain or be composed entirely of multifunctional envelope-type nanodevice (MENDs). MENDs may comprise condensed plasmid DNA, a PLL core, and a lipid film shell. A MEND may further comprise cell-penetrating peptide (e.g., stearyl octaarginine). The cell penetrating peptide may be in the lipid shell. The lipid envelope may be modified with one or more functional components, e.g., one or more of: polyethylene glycol (e.g., to increase vascular circulation time), ligands for targeting of specific tissues/cells, additional cell-penetrating peptides (e.g., for greater cellular delivery), lipids to enhance endosomal escape, and nuclear delivery tags. In some examples, the MEND may be a tetra-lamellar MEND (T-MEND), which may target the cellular nucleus and mitochondria. In certain examples, a MEND may be a PEG-peptide-DOPE-conjugated MEND (PPD-MEND), which may target bladder cancer cells. Examples of MENDs include those described in Kogure K, et al. (2004). J Control Release 98:317-23; Nakamura T, et al. (2012). Acc Chem Res 45:1113-21.

### Lipid-Coated Mesoporous Silica Particles

The delivery vehicles can contain or be composed entirely of lipid-coated mesoporous silica particles. Lipid-coated mesoporous silica particles may comprise a mesoporous silica nanoparticle core and a lipid membrane shell. The silica core may have a large internal surface area, leading to high cargo loading capacities. In some embodiments, pore sizes, pore chemistry, and overall particle sizes may be modified for loading different types of cargos. The lipid coating of the particle may also be modified to maximize cargo loading, increase circulation times, and provide precise targeting and cargo release. Examples of lipid-coated mesoporous silica particles include those described in Du X, et al. (2014). Biomaterials 35:5580-90; Durfee P N, et al. (2016). ACS Nano 10:8325-45.

### Inorganic Nanoparticles

The delivery vehicles can contain or be composed entirely of inorganic nanoparticles. Examples of inorganic nanoparticles include carbon nanotubes (CNTs) (e.g., as described in Bates K and Kostarelos K. (2013). Adv Drug Deliv Rev 65:2023-33.), bare mesoporous silica nanoparticles (MSNPs) (e.g., as described in Luo G F, et al. (2014). Sci Rep 4:6064), and dense silica nanoparticles (SiNPs) (as described in Luo D and Saltzman W M. (2000). Nat Biotechnol 18:893-5).

### Exosomes

The delivery vehicles can contain or be composed entirely of exosomes. Exosomes include membrane bound extracellular vesicles, which can be used to contain and delivery various types of biomolecules, such as proteins, carbohydrates, lipids, and nucleic acids, and complexes thereof (e.g., RNPs). Examples of exosomes include those described in Schroeder A, et al., J Intern Med. 2010 January; 267(1):9-21; E1-Andaloussi S, et al., Nat Protoc. 2012 December; 7(12):2112-26; Uno Y, et al., Hum Gene Ther. 2011 June; 22(6):711-9; Zou W, et al., Hum Gene Ther. 2011 April; 22(4):465-75.

In some examples, the exosome forms a complex (e.g., by binding directly or indirectly) to one or more components of the cargo. In certain examples, a molecule of an exosome may be fused with first adapter protein and a component of the cargo may be fused with a second adapter protein. The first and the second adapter protein may specifically bind each other, thus associating the cargo with the exosome. Examples of such exosomes include those described in Ye Y, et al., Biomater Sci. 2020 Apr. 28. doi: 10.1039/dObm00427h.

Other non-limiting, exemplary exosomes include any of those set forth in Alvarez-Erviti et al. 2011, Nat Biotechnol 29: 341; E1-Andaloussi et al. (Nature Protocols 7:2112-2126(2012); and Wahlgren et al. (Nucleic Acids Research, 2012, Vol. 40, No. 17 e130).

### Spherical Nucleic Acids (SNAs)

Spherical nucleic acids (SNA) are three-dimensional arrangements of nucleic acids, with densely packed and radially arranged oligonucleotides on a central nanoparticle core. In its simplest form the SNA is composed of oligonucleotides and a core. In some embodiments, the delivery vehicle can contain or be composed entirely of SNAs. SNAs are three dimensional nanostructures that can be composed of densely functionalized and highly oriented nucleic acids that can be covalently attached to the surface of spherical nanoparticle cores. The core may be a hollow core which is produced by a 3-dimensional arrangement of molecules which form the outer boundary of the core. For instance, the molecules may be in the form of a lipid layer or bilayer which has a hollow center. In other embodiments, the molecules may be in the form of lipids, such as amphipathic lipids, i.e., sterols which are linked to an end the oligonucleotide. Sterols such as cholesterol linked to an end of an oligonucleotide may associate with one another and form the outer edge of a hollow core with the oligonucleotides radiating outward from the core. The core may also be a solid or semi-solid core.

The oligonucleotides to be delivered can be associated with the core of an SNP. An oligonucleotide that is associated with the core may be covalently linked to the core or non-covalently linked to the core, i.e., potentially through hydrophobic interactions. For instance, when a sterol forms the outer edge of the core an oligonucleotide may be covalently linked to the sterol directly or indirectly. When a lipid layer forms the core, the oligonucleotide may be covalently linked to the lipid or may be non-covalently linked to the lipids e.g., by interactions with the oligonucleotide or a molecule such as a cholesterol attached to the oligonucleotide directly or indirectly through a linker.

A spherical nucleic acid (SNA) can be functionalized in order to attach a polynucleotide. Alternatively or additionally, the polynucleotide can be functionalized. One mechanism for functionalization is the alkanethiol method, whereby oligonucleotides are functionalized with alkanethiols at their 3′ or 5′ termini prior to attachment to gold nanoparticles or nanoparticles comprising other metals, semiconductors or magnetic materials. Such methods are described, for example Whitesides, Proceedings of the Robert A. Welch Foundation 39th Conference On Chemical Research Nanophase Chemistry, Houston, Tex., pages 109-121 (1995), and Mucic et al. Chem. Commun. 555-557 (1996). Oligonucleotides can also be attached to nanoparticles using other functional groups such as phosophorothioate groups, as described in and incorporated by reference from U.S. Pat. No. 5,472,881, or substituted alkylsiloxanes, as described in and incorporated by reference from Burwell, Chemical Technology, 4, 370-377 (1974) and Matteucci and Caruthers, J. Am. Chem. Soc., 103, 3185-3191 (1981). In some instances, oligonucleotides are attached to nanoparticles by terminating the polynucleotide with a 5′ or 3′ thionucleoside. In other instances, an aging process is used to attach oligonucleotides to nanoparticles as described in and incorporated by reference from U.S. Pat. Nos. 6,361,944, 6,506,569, 6,767,702 and 6,750,016 and PCT Publication Nos. WO 1998/004740, WO 2001/000876, WO 2001/051665 and WO 2001/073123. In some embodiments, the core is a metal core. In some embodiments, the core is an inorganic metal core. In some embodiments, the core is a gold core.

In some instances, the oligonucleotide is attached or inserted in the SNA. A spacer sequence can be included between the attachment site and the oligonucleotide. In some embodiments, a spacer sequence comprises or consists of an oligonucleotide, a peptide, a polymer or an oligoethylene glycol. In a preferred embodiment, the spacer is oligoethylene glycol and more preferably, hexaethyleneglycol.

Non-limiting, exemplary SNAs can be any of those set forth in Cutler et al., J. Am. Chem. Soc. 2011 133:9254-9257, Hao et al., Small. 2011 7:3158-3162, Zhang et al., ACS Nano. 2011 5:6962-6970, Cutler et al., J. Am. Chem. Soc. 2012 134:1376-1391, Young et al., Nano Lett. 2012 12:3867-71, Zheng et al., Proc. Natl. Acad. Sci. USA. 2012 109:11975-80, Mirkin, Nanomedicine 2012 7:635-638 Zhang et al., J. Am. Chem. Soc. 2012 134:16488-1691, Weintraub, Nature 2013 495:S14-S16, Choi et al., Proc. Natl. Acad. Sci. USA. 2013 110(19):7625-7630, Jensen et al., Sci. Transl. Med. 5, 209ra152 (2013) and Mirkin, et al., U.S. Pat. App. Pub. US20210002640 and US20200188521.

### Self-Assembling Nanoparticles

In some embodiments, the delivery vehicle contains or is composed entirely of a self-assembling nanoparticle. The self-assembling nanoparticles can contain one or more polymers. The self-assembling nanoparticles can be PEGylated. Self-assembling nanoparticles are known in the art. Non-limiting, exemplary self-assembling nanoparticles can any as set forth in Schiffelers et al., Nucleic Acids Research, 2004, Vol. 32, No. 19, Bartlett et al. (PNAS, Sep. 25, 2007, vol. 104, no. 39; Davis et al., Nature, Vol 464, 15 Apr. 2010.

### Supercharged Proteins

In some embodiments, the delivery vehicle contains or is composed entirely of supercharged protein. As used herein “Supercharged proteins” are a class of engineered or naturally occurring proteins with unusually high positive or negative net theoretical charge. Non-limiting, exemplary supercharged proteins can be any of those set forth in Lawrence et al., 2007, Journal of the American Chemical Society 129, 10110-10112.

### Agrobacterium

In some embodiments, delivery and/or transformation of, particularly, a plant cell, is via Agrobacterium mediated transformation. The DNA constructs (such as those containing an engineered polynucleotide of the present disclosure) can be combined with suitable T-DNA flanking regions and introduced into a conventional Agrobacterium tumefaciens host vector. The foreign DNA can be incorporated into the genome of plants by infecting the plants or by incubating plant protoplasts with Agrobacterium bacteria, containing one or more Ti (tumor-inducing) plasmids. (see e.g., Fraley et al., (1985), Rogers et al., (1987) and U.S. Pat. No. 5,563,055).

### Targeted Delivery

In some embodiments, the delivery vehicle is configured for targeted delivery to a specific cell, tissue, organ, or system. In such embodiments, the delivery vehicle can include one or more targeting moieties that can direct targeted delivery of the cargo(s). In an embodiment, the delivery vehicle comprises a targeting moiety, such as on its surface. Exemplary targeting moieties include, without limitation, small molecule, polypeptide, and/or polynucleotide ligands for cell surface molecules, antibodies, affibodies, aptamers, or any combination thereof. In some embodiments, a targeted delivery vehicle can be generated by coupling, conjugating, attaching, or otherwise associating a targeting moiety with a delivery vehicle described elsewhere herein. In some embodiments, multiple targeting moieties with different targets are coupled to a delivery vehicle. In some embodiments a multivalent approach can be employed. Multivalent presentation of targeting moieties (e.g., antibodies) can also increase the uptake and signaling properties of targeting moiety fragments. In some embodiments, targeted delivery can be to one cell type or to multiple cell types. Methods of coupling conjugating, attaching, or otherwise associating a targeting moiety with a delivery vehicle are generally known in the art.

In some embodiments, the targeting moiety is an aptamer. Aptamers are ssDNA or RNA oligonucleotides that impart high affinity and specific recognition of the target molecules by electrostatic interactions, hydrogen bonding and hydrophobic interactions as opposed to the Watson-Crick base pairing, which is typical for the bonding interactions of oligonucleotides. Aptamers as a targeting moiety can have advantages over antibodies: aptamers can demonstrate higher target antigen recognition as compared with antibodies; aptamers can be more stable and smaller in size as compared with antibodies; aptamers can be easily synthesized and chemically modified for molecular conjugation; and aptamers can be changed in sequence for improved selectivity and can be developed to recognize poorly immunogenic targets.

Targeted delivery includes intracellular delivery. Delivery vehicles that utilize the endocytic pathway are entrapped in the endosomes (pH 6.5-6) and subsequently fuse with lysosomes (pH<5), where they undergo degradation that results in a lower therapeutic potential. The low endosomal pH can be taken advantage of to escape degradation. Fusogenic lipids or peptides, which destabilize the endosomal membrane after the conformational transition/activation at a lowered pH can be included in the delivery vehicle. Such lipids or peptides can include amines, which are protonated at an acidic pH and cause endosomal swelling and rupture by a buffer effect, pore-forming protein listeriolysin 0, histidine-rich peptides have the ability to fuse with the endosomal membrane, resulting in pore formation, and can buffer the proton pump causing membrane lysis, and/or unsaturated dioleoylphosphatidylethanolamine (DOPE) that readily adopt an inverted hexagonal shape at a low pH and causes fusion of liposomes to the endosomal membrane. Inclusion of such molecules can result in an efficient endosomal release and/or may provide an endosomal escape mechanism to increase cargo delivery by the vehicle.

In some embodiments, the delivery vehicle is or includes modified CPP(s) that can facilitate intracellular delivery via macropinocytosis followed by endosomal escape. CPPs are described in greater detail elsewhere herein.

In some embodiments, targeted delivery is organelle-specific targeted delivery. A delivery vehicle can be surface-functionalized with a targeting moiety that can direct organelle specific delivery, such as a nuclear localization sequence, ribosomal entry sequence, mitochondria specific moiety, and/or the like. The invention further comprehends a lipid entity of the invention targeting the nucleus, e.g., via a DNA-intercalating moiety.

In some embodiments, the targeted delivery is multifunctional targeted delivery that can be accomplished by attaching more than one targeting moiety to the surface of the delivery vehicle. In some embodiments, such an enhances accumulation in a desired site and/or promotes organelle-specific delivery and/or target a particular type of cell and/or respond to the local environmental stimuli such as temperature (e.g., elevated), pH (e.g., acidic or basic), respond to targeted or localized externally applied stimuli such as a magnetic field, light, energy, heat or ultrasound (e.g., responsive delivery, which is described in greater detail elsewhere herein) and/or promote intracellular delivery of the cargo.

Exemplary targeting moieties are generally known in the art, and include without limitation, those described in e.g., in e.g., Deshpande et al, “Current trends in the use of liposomes for tumor targeting,” Nanomedicine (Lond). 8(9), doi:10.2217/nnm.13.118 (2013), International Patent Publication No. WO 2016/027264, Lorenzer et al, “Going beyond the liver: Progress and challenges of targeted delivery of siRNA therapeutics,” Journal of Controlled Release, 203: 1-15 (2015); Surace et al, “Lipoplexes targeting the CD44 hyaluronic acid receptor for efficient transfection of breast cancer cells,” J. Mol Pharm 6(4):1062-73; doi: 10.1021/mp800215d (2009); Sonoke et al, “Galactose-modified cationic liposomes as a liver-targeting delivery system for small interfering RNA,” Biol Pharm Bull. 34(8):1338-42 (2011); Torchilin, “Antibody-modified liposomes for cancer chemotherapy,” Expert Opin. Drug Deliv. 5 (9), 1003-1025 (2008); Manjappa et al, “Antibody derivatization and conjugation strategies: application in preparation of stealth immunoliposome to target chemotherapeutics to tumor,” J. Control. Release 150 (1), 2-22 (2011); Sofou S “Antibody-targeted liposomes in cancer therapy and imaging,” Expert Opin. Drug Deliv. 5 (2): 189-204 (2008); Gao J et al, “Antibody-targeted immunoliposomes for cancer treatment,” Mini. Rev. Med. Chem. 13(14): 2026-2035 (2013); Molavi et al, “Anti-CD30 antibody conjugated liposomal doxorubicin with significantly improved therapeutic efficacy against anaplastic large cell lymphoma,” Biomaterials 34(34):8718-25 (2013), Zhao et al., 2020. Cell 181:151-167, particularly at tables 1-5; Liu et al., Front. Bioeng. Biotechnol. 2021. 9:701504. doi: 10.3389/fbioe.2021.701504; US20210379192 (describes exemplary skeletal muscle cell targeting moieties), Snow-Lisy et al., Drug. Deliv. Transl. Res. 1:351(2011); US20060263336 (describes exemplary progenitor cell targeting moieties) each of which and the documents cited therein are hereby incorporated herein by reference.

Other exemplary targeting moieties are described elsewhere herein, such as epitope tags, reporter and selectable markers, and/or the like which can be configured for and/or operate in some embodiments as targeting moieties.

### Responsive Delivery

In some embodiments, the delivery vehicle can allow for responsive delivery of the cargo(s). Responsive delivery, as used in this context herein, refers to delivery of cargo(s) by the delivery vehicle in response to an external stimuli. Examples of suitable stimuli include, without limitation, an energy (light, heat, cold, and the like), a chemical stimuli (e.g., chemical composition, etc.), and a biologic or physiologic stimuli (e.g., environmental pH, osmolarity, salinity, biologic molecule, etc.). In some embodiments, a targeting moiety is responsive to an external stimuli and facilitate responsive delivery. In other embodiments, responsiveness is determined by a non-targeting moiety component of the delivery vehicle.

In some embodiments, the responsive delivery is stimuli-sensitive, e.g., sensitive to an externally applied stimuli, such as magnetic fields, ultrasound or light; and pH-triggering can also be used, e.g., a labile linkage can be used between a hydrophilic moiety such as PEG and a hydrophobic moiety such as a lipid entity of the invention, which is cleaved only upon exposure to the relatively acidic conditions characteristic of the a particular environment or microenvironment such as an endocytic vacuole or the acidotic tumor mass. pH-sensitive copolymers can also be incorporated in embodiments of the invention can provide shielding; diortho esters, vinyl esters, cysteine-cleavable lipopolymers, double esters and hydrazones are a few examples of pH-sensitive bonds that are quite stable at pH 7.5, but are hydrolyzed relatively rapidly at pH 6 and below, e.g., a terminally alkylated copolymer of N-isopropylacrylamide and methacrylic acid that copolymer facilitates destabilization of a lipid entity of the invention and release in compartments with decreased pH value; or, the invention comprehends ionic polymers for generation of a pH-responsive lipid entity of the invention (e.g., poly(methacrylic acid), poly(diethylaminoethyl methacrylate), poly(acrylamide) and poly(acrylic acid)).

In some embodiments, the responsive delivery is temperature-triggered delivery. Many pathological areas, such as inflamed tissues and tumors, show a distinctive hyperthermia compared with normal tissues. Utilizing this hyperthermia is an attractive strategy in cancer therapy since hyperthermia is associated with increased tumor permeability and enhanced uptake. This technique involves local heating of the site to increase microvascular pore size and blood flow, which, in turn, can result in an increased extravasation of embodiments of the invention. Temperature-sensitive lipid entity of the invention can be prepared from thermosensitive lipids or polymers with a low critical solution temperature. Above the low critical solution temperature (e.g., at site such as tumor site or inflamed tissue site), the polymer precipitates, disrupting the liposomes to release. Lipids with a specific gel-to-liquid phase transition temperature are used to prepare these lipid entities of the invention; and a lipid for a thermosensitive embodiment can be dipalmitoylphosphatidylcholine. Thermosensitive polymers can also facilitate destabilization followed by release, and a useful thermosensitive polymer is poly (N-isopropylacrylamide). Another temperature triggered system can employ lysolipid temperature-sensitive liposomes.

In some embodiments, the responsive delivery is redox-triggered delivery. The difference in redox potential between normal and inflamed or tumor tissues, and between the intra- and extra-cellular environments has been exploited for delivery, e.g., GSH is a reducing agent abundant in cells, especially in the cytosol, mitochondria and nucleus. The GSH concentrations in blood and extracellular matrix are just one out of 100 to one out of 1000 of the intracellular concentration, respectively. This high redox potential difference caused by GSH, cysteine and other reducing agents can break the reducible bonds, destabilize a lipid entity of the invention and result in release of payload. The disulfide bond can be used as the cleavable/reversible linker in a lipid entity of the invention, because it causes sensitivity to redox owing to the disulfideto-thiol reduction reaction; a lipid entity of the invention can be made reduction sensitive by using two (e.g., two forms of a disulfide-conjugated multifunctional lipid as cleavage of the disulfide bond (e.g., via tris(2-carboxyethyl)phosphine, dithiothreitol, L-cysteine or GSH), can cause removal of the hydrophilic head group of the conjugate and alter the membrane organization leading to release of payload. Calcein release from reduction-sensitive lipid entity of the invention containing a disulfide conjugate can be more useful than a reduction-insensitive embodiment.

Enzymes can also be used as a trigger to release payload. Enzymes, including MMPs (e.g., MMP2), phospholipase A2, alkaline phosphatase, transglutaminase or phosphatidylinositol-specific phospholipase C, have been found to be overexpressed in certain tissues, e.g., tumor tissues. In the presence of these enzymes, specially engineered enzyme-sensitive lipid entity of the invention can be disrupted and release the payload. an MMP2-cleavable octapeptide (Gly-Pro-Leu-Gly-Ile-Ala-Gly-Gln (SEQ ID NO: 22)) can be incorporated into a linker, and can have antibody targeting, e.g., antibody 2C5.

In some embodiments, the responsive delivery is light- or energy-triggered delivery, e.g., the lipid entity of the invention can be light-sensitive, such that light or energy can facilitate structural and conformational changes, which lead to direct interaction of the lipid entity of the invention with the target cells via membrane fusion, photo-isomerism, photofragmentation or photopolymerization; such a moiety therefor can be benzoporphyrin photosensitizer. Ultrasound can be a form of energy to trigger delivery; a lipid entity of the invention with a small quantity of particular gas, including air or perfluorated hydrocarbon can be triggered to release with ultrasound, e.g., low-frequency ultrasound (LFUS). Magnetic delivery: A lipid entity of the invention can be magnetized by incorporation of magnetites, such as Fe3O4 or γ-Fe2O3, e.g., those that are less than 10 nm in size. Targeted delivery can be then by exposure to a magnetic field.

### Cells and Organisms

Described in certain example embodiments herein are cell(s) comprising a nudiviral nucleic acid of the present description; an engineered polynucleotide of the present description; a vector or vector system of the present description; or any combination thereof. In certain example embodiments, the cell is a prokaryotic or eukaryotic cell. In certain example embodiments, the cell is a shrimp cell, fish cell, insect cell, or a plant cell. In certain example embodiments, the cell is a mammalian cell, optionally a human cell. Described in certain example embodiments herein are cell population comprising one or more cells of the present description.

Described in certain example embodiments herein are organisms comprising a nudiviral nucleic acid of the present description; an engineered polynucleotide of the present description; a vector or vector system of the present description; a cell or cell population as in the present description; or any combination thereof. Described in certain example embodiments, the organism is a non-human animal, insect, or a plant. In certain example embodiments herein, the organism is a crustacean or fish.

### Cells

Also described herein are modified cells and progeny thereof that contain an engineered nudiviral nucleic acid, engineered polynucleotide, and/or vector or vector system of the present disclosure. In some embodiments, the cell is a eukaryotic cell. In some embodiments, the eukaryotic cell is a mammalian cell. In some embodiments, the eukaryotic cell is a non-human mammalian cell. In some embodiments, the cell is a human cell. In some embodiments, the cell is a plant cell. In some embodiments, the cell is a fungal cell. In some embodiments, the cell is a prokaryotic cell. The cells can be modified in vitro, ex vivo, or in vivo. The cells can be modified by delivering a polynucleotide modifying agent or system described in greater detail elsewhere herein or a component thereof into a cell by a suitable delivery mechanism. Suitable delivery methods and techniques include but are not limited to, transfection via a vector, transduction with viral particles, electroporation, endocytic methods, and others, which are described elsewhere herein and will be appreciated by those of ordinary skill in the art in view of this disclosure.

The cells can be further optionally cultured and/or expanded in vitro or ex vivo using any suitable cell culture techniques or conditions, which unless specified otherwise herein, will be appreciated by one of ordinary skill in the art in view of this disclosure. In some embodiments, the cells can be modified, optionally cultured and/or expanded, and/or administered to a subject in need thereof. In some embodiments, cells can be isolated from a subject, subsequently modified and optionally cultured and/or expanded, and administered back to the subject. Such administration can be referred to as autologous administration. In some embodiments, cells can be isolated from a first subject, subsequently modified, optionally cultured and/or expanded, and administered to a second subject, where the first subject and the second subject are different. Such administration can be referred to as non-autologous administration.

### Organisms

Also described herein are modified organisms and progeny thereof that contain an engineered nudiviral nucleic acid, engineered polynucleotide, and/or vector or vector system of the present disclosure. In some embodiments, the modified organisms can include one or more modified cells as are described elsewhere herein. In some embodiments, the modified organism is a non-human mammal. In some embodiments, the modified organism is a modified plant. In some embodiments, the modified organism is an insect. In some embodiments, the modified organism is a fungus. In some embodiments, the modified organism is a fungus. The modified organisms can be generated using a that can be modified by an embodiment of the engineered or non-natural guided excision-transposition system described herein. Methods of making modified organisms are described in greater detail elsewhere herein.

The systems and methods described herein can be used in non-animal organisms, e.g., plants, fungi to generated modified non-animal organisms. The system and methods described can be used to generate non-human animal organisms. The system and methods described herein can be used to modify non-germline cells in a human. In some embodiments, the modification is expression of a polynucleotide of interest, gene of interest, and/or allele of interest, such as that whose expression is driven by a nudiviral nucleic acid of the present disclosure.

### Non-Animal Cells and Organisms

In some embodiments, the modified cell or organism is a non-animal cell or organism such as plants, yeast, etc. In general, the term “plant” relates to any various photosynthetic, eukaryotic, unicellular or multicellular organism of the kingdom Plantae characteristically growing by cell division, containing chloroplasts, and having cell walls comprised of cellulose. The term plant encompasses monocotyledonous and dicotyledonous plants. Exemplary plants within the scope of this disclosure include, without limitation, angiosperm and gymnosperm plants such as acacia, alfalfa, amaranth, apple, apricot, artichoke, ash tree, asparagus, avocado, banana, barley, beans, beet, birch, beech, blackberry, blueberry, broccoli, Brussel's sprouts, cabbage, canola, cantaloupe, carrot, cassava, cauliflower, cedar, a cereal, celery, chestnut, cherry, Chinese cabbage, citrus, clementine, clover, coffee, corn, cotton, cowpea, cucumber, cypress, eggplant, elm, endive, eucalyptus, fennel, figs, fir, geranium, grape, grapefruit, groundnuts, ground cherry, gum hemlock, hickory, kale, kiwifruit, kohlrabi, larch, lettuce, leek, lemon, lime, locust, pine, maidenhair, maize, mango, maple, melon, millet, mushroom, mustard, nuts, oak, oats, oil palm, okra, onion, orange, an ornamental plant or flower or tree, papaya, palm, parsley, parsnip, pea, peach, peanut, pear, peat, pepper, persimmon, pigeon pea, pine, pineapple, plantain, plum, pomegranate, potato, pumpkin, radicchio, radish, rapeseed, raspberry, rice, rye, sorghum, safflower, sallow, soybean, spinach, spruce, squash, strawberry, sugar beet, sugarcane, sunflower, sweet potato, sweet corn, tangerine, tea, tobacco, tomato, trees, triticale, turf grasses, turnips, vine, walnut, watercress, watermelon, wheat, yams, yew, and zucchini. The term plant also encompasses Algae, which are mainly photoautotrophs unified primarily by their lack of roots, leaves and other organs that characterize higher plants.

Additional exemplary plants and plant cells included within the scope of the present disclosure include, but are not limited to, those monocotyledonous and dicotyledonous plants, such as crops including grain crops (e.g., wheat, maize, rice, millet, barley), fruit crops (e.g., tomato, apple, pear, strawberry, orange), forage crops (e.g., alfalfa), root vegetable crops (e.g., carrot, potato, sugar beets, yam), leafy vegetable crops (e.g., lettuce, spinach); flowering plants (e.g., petunia, rose, chrysanthemum), conifers and pine trees (e.g., pine fir, spruce); plants used in phytoremediation (e.g., heavy metal accumulating plants); oil crops (e.g., sunflower, rape seed) and plants used for experimental purposes (e.g., Arabidopsis). Plant cells and tissues for engineering include, without limitation, roots, stems, leaves, flowers, and reproductive structures, undifferentiated meristematic cells, parenchyma, collenchyma, sclerenchyma, xylem, phloem, epidermis, and germplasm. Thus, the methods and modifying agents and systems described herein can be used over a broad range of plants, such as for example with dicotyledonous plants belonging to the orders Magniolales, Illiciales, Laurales, Piperales, Aristochiales, Nymphaeales, Ranunculales, Papeverales, Sarraceniaceae, Trochodendrales, Hamamelidales, Eucomiales, Leitneriales, Myricales, Fagales, Casuarinales, Caryophyllales, Batales, Polygonales, Plumbaginales, Dilleniales, Theales, Malvales, Urticales, Lecythidales, Violales, Salicales, Capparales, Ericales, Diapensales, Ebenales, Primulales, Rosales, Fabales, Podostemales, Haloragales, Myrtales, Cornales, Proteales, San tales, Rafflesiales, Celastrales, Euphorbiales, Rhamnales, Sapindales, Juglandales, Geraniales, Polygalales, Umbellales, Gentianales, Polemoniales, Lamiales, Plantaginales, Scrophulariales, Campanulales, Rubiales, Dipsacales, and Asterales; the methods and CRISPR-Cas systems can be used with monocotyledonous plants such as those belonging to the orders Alismatales, Hydrocharitales, Najadales, Triuridales, Commelinales, Eriocaulales, Restionales, Poales, Juncales, Cyperales, Typhales, Bromeliales, Zingiberales, Arecales, Cyclanthales, Pandanales, Arales, Lilliales, and Orchid ales, or with plants belonging to Gymnospermae, e.g those belonging to the orders Pinales, Ginkgoales, Cycadales, Araucariales, Cupressales and Gnetales.

Further exemplary plants and plant cells included within the scope of the present disclosure include, but are not limited to, dicot, monocot or gymnosperm genera hereunder: Atropa, Alseodaphne, Anacardium, Arachis, Beilschmiedia, Brassica, Carthamus, Cocculus, Croton, Cucumis, Citrus, Citrullus, Capsicum, Catharanthus, Cocos, Coffea, Cucurbita, Daucus, Duguetia, Eschscholzia, Ficus, Fragaria, Glaucium, Glycine, Gossypium, Helianthus, Hevea, Hyoscyamus, Lactuca, Landolphia, Linum, Litsea, Lycopersicon, Lupinus, Manihot, Majorana, Malus, Medicago, Nicotiana, Olea, Parthenium, Papaver, Persea, Phaseolus, Pistacia, Pisum, Pyrus, Prunus, Raphanus, Ricinus, Senecio, Sinomenium, Stephania, Sinapis, Solanum, Theobroma, Trifolium, Trigonella, Vicia, Vinca, Vilis, and Vigna; and the genera Allium, Andropogon, Aragrostis, Asparagus, Avena, Cynodon, Elaeis, Festuca, Festulolium, Heterocallis, Hordeum, Lemna, Lolium, Musa, Oryza, Panicum, Pannesetum, Phleum, Poa, Secale, Sorghum, Triticum, Zea, Abies, Cunninghamia, Ephedra, Picea, Pinus, and Pseudotsuga.

Further exemplary non-animal cells included within the scope of the present disclosure include, but are not limited to, “algae” or “algae cells”; including for example algea selected from several eukaryotic phyla, including the Rhodophyta (red algae), Chlorophyta (green algae), Phaeophyta (brown algae), Bacillariophyta (diatoms), Eustigmatophyta and dinoflagellates as well as the prokaryotic phylum Cyanobacteria (blue-green algae). The term “algae” includes for example algae selected from: Amphora, Anabaena, Anikstrodesmis, Botryococcus, Chaetoceros, Chlamydomonas, Chlorella, Chlorococcum, Cyclotella, Cylindrotheca, Dunaliella, Emiliana, Euglena, Hematococcus, Isochrysis, Monochrysis, Monoraphidium, Nannochloris, Nannnochloropsis, Navicula, Nephrochloris, Nephroselmis, Nitzschia, Nodularia, Nostoc, Oochromonas, Oocystis, Oscillartoria, Pavlova, Phaeodactylum, Playtmonas, Pleurochrysis, Porhyra, Pseudoanabaena, Pyramimonas, Stichococcus, Synechococcus, Synechocystis, Tetraselmis, Thalassiosira, and Trichodesmium.

A part of a plant, e.g., a “plant tissue” may be treated according to the methods of the present invention to produce an improved plant. Plant tissue also encompasses plant cells. The term “plant cell” as used herein refers to individual units of a living plant, either in an intact whole plant or in an isolated form grown in in vitro tissue cultures, on media or agar, in suspension in a growth media or buffer or as a part of higher organized unites, such as, for example, plant tissue, a plant organ, or a whole plant.

A “protoplast” refers to a plant cell that has had its protective cell wall completely or partially removed using, for example, mechanical or enzymatic means resulting in an intact biochemical competent unit of living plant that can reform their cell wall, proliferate and regenerate grow into a whole plant under proper growing conditions.

The term “transformation” broadly refers to the process by which a plant host is genetically modified by the introduction of DNA by means of Agrobacteria or one of a variety of chemical or physical methods. As used herein, the term “plant host” refers to plants, including any cells, tissues, organs, or progeny of the plants. Many suitable plant tissues or plant cells can be transformed and include, but are not limited to, protoplasts, somatic embryos, pollen, leaves, seedlings, stems, calli, stolons, microtubers, and shoots. A plant tissue also refers to any clone of such a plant, seed, progeny, propagule whether generated sexually or asexually, and descendants of any of these, such as cuttings or seed.

The term “progeny”, such as the progeny of a transgenic plant, is one that is born of, begotten by, or derived from a plant or the transgenic plant. The introduced DNA molecule may also be transiently introduced into the recipient cell such that the introduced DNA molecule is not inherited by subsequent progeny and thus not considered “transgenic”. Accordingly, as used herein, a “non-transgenic” plant or plant cell is a plant which does not contain a foreign DNA stably integrated into its genome. The progeny may be a clone of the produced plant or animal, or may result from sexual reproduction by crossing with other individuals of the same species to introgress further desirable traits into their offspring. The cell may be in vivo or ex vivo in the cases of multicellular organisms, particularly plant. The term plant and plant progeny includes, gametes, seeds, germplasm, embryos, either zygotic or somatic, progeny or hybrids of plants comprising the genetic modification, which are produced by traditional breeding methods, are also included within the scope of the present invention. Such plants may contain a heterologous or foreign DNA sequence inserted at or instead of a target sequence. Alternatively, such plants may contain only an alteration (mutation, deletion, insertion, substitution) in one or more nucleotides. As such, such plants will only be different from their progenitor plants by the presence of the particular modification.

Yeast cells may include budding yeast cells, fission yeast cells, and mold cells. Without being limited to these organisms, many types of yeast used in laboratory and industrial settings are part of the phylum Ascomycota. In some embodiments, the yeast cell is an S. cerervisiae, Kluyveromyces marxianus, or Issatchenkia orientalis cell. Other yeast cells may include without limitation Candida spp. (e.g., Candida albicans), Yarrowia spp. (e.g., Yarrowia lipolytica), Pichia spp. (e.g., Pichia pastoris), Kluyveromyces spp. (e.g., Kluyveromyces lactis and Kluyveromyces marxianus), Neurospora spp. (e.g., Neurospora crassa), Fusarium spp. (e.g., Fusarium oxysporum), and Issatchenkia spp. (e.g., Issatchenkia orientalis, a.k.a. Pichia kudriavzevii and Candida acidothermophilum). In some embodiments, the fungal cell is a filamentous fungal cell. As used herein, the term “filamentous fungal cell” refers to any type of fungal cell that grows in filaments, i.e., hyphae or mycelia. Examples of filamentous fungal cells may include without limitation Aspergillus spp. (e.g., Aspergillus niger), Trichoderma spp. (e.g., Trichoderma reesei), Rhizopus spp. (e.g., Rhizopus oryzae), and Mortierella spp. (e.g., Mortierella isabellina).

In some embodiments, the fungal cell is an industrial strain. As used herein, “industrial strain” refers to any strain of fungal cell used in or isolated from an industrial process, e.g., production of a product on a commercial or industrial scale. Industrial strain may refer to a fungal species that is typically used in an industrial process, or it may refer to an isolate of a fungal species that may be also used for non-industrial purposes (e.g., laboratory research). Examples of industrial processes may include fermentation (e.g., in production of food or beverage products), distillation, biofuel production, production of a compound, and production of a polypeptide. Examples of industrial strains may include, without limitation, JAY270 and ATCC4124.

In some embodiments, the fungal cell is a polyploid cell. As used herein, a “polyploid” cell may refer to any cell whose genome is present in more than one copy. A polyploid cell may refer to a type of cell that is naturally found in a polyploid state, or it may refer to a cell that has been induced to exist in a polyploid state (e.g., through specific regulation, alteration, inactivation, activation, or modification of meiosis, cytokinesis, or DNA replication). A polyploid cell may refer to a cell whose entire genome is polyploid, or it may refer to a cell that is polyploid in a particular genomic locus of interest. Without wishing to be bound to theory, it is thought that the abundance of guideRNA may more often be a rate-limiting component in genome engineering of polyploidy cells than in haploid cells, and thus the methods using the systems described herein may take advantage of using a certain fungal cell type.

In some embodiments, the fungal cell is a diploid cell. As used herein, a “diploid” cell may refer to any cell whose genome is present in two copies. A diploid cell may refer to a type of cell that is naturally found in a diploid state, or it may refer to a cell that has been induced to exist in a diploid state (e.g., through specific regulation, alteration, inactivation, activation, or modification of meiosis, cytokinesis, or DNA replication). For example, the S. cerevisiae strain S228C may be maintained in a haploid or diploid state. A diploid cell may refer to a cell whose entire genome is diploid, or it may refer to a cell that is diploid in a particular genomic locus of interest. In some embodiments, the fungal cell is a haploid cell. As used herein, a “haploid” cell may refer to any cell whose genome is present in one copy. A haploid cell may refer to a type of cell that is naturally found in a haploid state, or it may refer to a cell that has been induced to exist in a haploid state (e.g., through specific regulation, alteration, inactivation, activation, or modification of meiosis, cytokinesis, or DNA replication). For example, the S. cerevisiae strain S228C may be maintained in a haploid or diploid state. A haploid cell may refer to a cell whose entire genome is haploid, or it may refer to a cell that is haploid in a particular genomic locus of interest. Additional exemplary fungal and yeast cells are described elsewhere herein.

### Non-Human Animals and Insects

In some embodiments, the organism is a modified non-human animal. Exemplary modified animals and insects include, without limitation, non-human mammals, avians, crustaceans (shrimp, lobster, crab, crawfish, etc.), fish, reptiles, amphibians, and/or the like. In some embodiments, the non-human animal is a vertebrate. In some embodiments, the non-human animal is an invertebrate. Methods of introducing exogenous constructs into non-human animals is generally known in the art. See e.g., (reviewed in Sid and Schusser et al 2018. Front. Genet. Doi.org/10.3389/fgene.2018.00456) and other avians (e.g., Scott et al. 2010. ILAR J. 51(4):353-361), cattle (Yum et al., 2016. Scientific Reports. 6:27185 and Tait-Burkard et al. 2018. Genome Biology. 19:2014.), sheep and goats (see e.g., Kalds et al., 2019. Front. Genet. Doi.org//10.3389/fgene.2019.00750), horses (see e.g., West and Gill. 2016. J. Equine Vet. Sci. 41:1-6), dogs (see e.g., D. Duan. Nature Biomedical Engineering. 2018. 2: 795-796), reptiles (see e.g., Rasys et al. 2019. Cell Reports. 28:2288-2292), fish (including but not limited to zebrafish, see e.g., Datsomor et al. 2019. Scientific Reports. 9:7533, Liu et al. 2019. Front. Cell. Dev. Biol. https://doi.org/10.3389/fcell.2019.00013), insects (see e.g., Kotwica-Rolinska et al. 2019. Front. Physiol. https://doi.org/10.3389/fphys.2019.00891; Gantz and Akbari. 2018. Curr. Opin. Insect. Sci. 28:66-72), rabbits (see e.g., Kawano and Honda. 2017. Methods Mol. Biol. 4630:109-120; Liu et al., 2018. Nature Commun. 9:2717; and Liu et al. 2018. Gene. https://doi.org/10.1016/j.gene.2018.01.044), mice (see e.g., Hall et al. 2018. Curr Protoc Cell Biol. 81(1): e57), rats (see e.g. Back et al. 2019. Neuron. 102(1):105-119), amphibians (see e.g., Nakayama et al. 2013. Genesis. 51(12):835-843), nematodes (see e.g., J. B. Lok. 2019. Front. Genet. https://doi.org/10.3389/fgene.2019.00656), molluscs (see e.g., Abe and Kuroda. 2019. Development. 146: dev175976 doi: 10.1242/dev.175976, geckos, shrimp and other crustaceans (see e.g., Gui et al. Genes Genomes Genetics: 6(11): 3757-3764), oysters (Yu et al. 2019; March Biotechnol (NY) 21(3):301-309. doi: 10.1007/s10126-019-09885-y), and sponges (see e.g., Revilla-i-Domingo et al. 2018. Genetics. 210(2)435-443), the teachings of which can be adapted for use with one or more of the modifying agent(s) and/or systems described herein to generate the modified non-human animal or cell thereof.

### Formulations

Described in certain example embodiments herein are formulations comprising a nudiviral nucleic acid of the present description; an engineered polynucleotide of the present description; a vector or vector system of the present description; a cell or cell population as in the present description; or any combination thereof, and a pharmaceutically acceptable carrier.

Also described herein are pharmaceutical formulations that can contain an amount, effective amount, and/or least effective amount, and/or therapeutically effective amount of one or more compounds, molecules, compositions, vectors, vector systems, cells, or a combination thereof (which are also referred to as the primary active agent or ingredient elsewhere herein) described in greater detail elsewhere herein and a pharmaceutically acceptable carrier or excipient. As used herein, “pharmaceutical formulation” refers to the combination of an active agent, compound, or ingredient with a pharmaceutically acceptable carrier or excipient, making the composition suitable for diagnostic, therapeutic, or preventive use in vitro, in vivo, or ex vivo. As used herein, “pharmaceutically acceptable carrier or excipient” refers to a carrier or excipient that is useful in preparing a pharmaceutical formulation that is generally safe, non-toxic, and is neither biologically or otherwise undesirable, and includes a carrier or excipient that is acceptable for veterinary use as well as human pharmaceutical use. A “pharmaceutically acceptable carrier or excipient” as used in the specification and claims includes both one and more than one such carrier or excipient. When present, the compound can optionally be present in the pharmaceutical formulation as a pharmaceutically acceptable salt.

In some embodiments, the active ingredient is present as a pharmaceutically acceptable salt of the active ingredient. As used herein, “pharmaceutically acceptable salt” refers to any acid or base addition salt whose counter-ions are non-toxic to the subject to which they are administered in pharmaceutical doses of the salts. Suitable salts include, hydrobromide, iodide, nitrate, bisulfate, phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, malonate, mandelate, malate, phthalate, and pamoate.

The pharmaceutical formulations described herein can be administered to a subject in need thereof via any suitable method or route to a subject in need thereof. Suitable administration routes can include, but are not limited to auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratympanic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease to be treated and/or the active ingredient(s).

Where appropriate, compounds, molecules, compositions, vectors, vector systems, cells, or a combination thereof described in greater detail elsewhere herein can be provided to a subject in need thereof as an ingredient, such as an active ingredient or agent, in a pharmaceutical formulation. As such, also described are pharmaceutical formulations containing one or more of the compounds and salts thereof, or pharmaceutically acceptable salts thereof described herein. Suitable salts include, hydrobromide, iodide, nitrate, bisulfate, phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, malonate, mandelate, malate, phthalate, and pamoate.

As used herein, “agent” refers to any substance, compound, molecule, and the like, which can be biologically active or otherwise can induce a biological and/or physiological effect on a subject to which it is administered to. As used herein, “active agent” or “active ingredient” refers to a substance, compound, or molecule, which is biologically active or otherwise, induces a biological or physiological effect on a subject to which it is administered to. In other words, “active agent” or “active ingredient” refers to a component or components of a composition to which the whole or part of the effect of the composition is attributed. An agent can be a primary active agent, or in other words, the component(s) of a composition to which the whole or part of the effect of the composition is attributed. An agent can be a secondary agent, or in other words, the component(s) of a composition to which an additional part and/or other effect of the composition is attributed.

### Pharmaceutically Acceptable Carriers and Secondary Ingredients and Agents

The pharmaceutical formulation can include a pharmaceutically acceptable carrier. Suitable pharmaceutically acceptable carriers include, but are not limited to water, salt solutions, alcohols, gum arabic, vegetable oils, benzyl alcohols, polyethylene glycols, gelatin, carbohydrates such as lactose, amylose or starch, magnesium stearate, talc, silicic acid, viscous paraffin, perfume oil, fatty acid esters, hydroxy methylcellulose, and polyvinyl pyrrolidone, which do not deleteriously react with the active composition.

The pharmaceutical formulations can be sterilized, and if desired, mixed with agents, such as lubricants, preservatives, stabilizers, wetting agents, emulsifiers, salts for influencing osmotic pressure, buffers, coloring, flavoring and/or aromatic substances, and the like which do not deleteriously react with the active compound.

In some embodiments, the pharmaceutical formulation can also include an effective amount of secondary active agents, including but not limited to, biologic agents or molecules including, but not limited to, e.g., polynucleotides, amino acids, peptides, polypeptides, antibodies, aptamers, ribozymes, hormones, immunomodulators, antipyretics, anxiolytics, antipsychotics, analgesics, antispasmodics, anti-inflammatories, anti-histamines, anti-infectives, chemotherapeutics, and combinations thereof.

### Effective Amounts

In some embodiments, the amount of the primary active agent and/or optional secondary agent can be an effective amount, least effective amount, and/or therapeutically effective amount. As used herein, “effective amount” refers to the amount of the primary and/or optional secondary agent included in the pharmaceutical formulation that achieve one or more therapeutic effects or desired effect. As used herein, “least effective” amount refers to the lowest amount of the primary and/or optional secondary agent that achieves the one or more therapeutic or other desired effects. As used herein, “therapeutically effective amount” refers to the amount of the primary and/or optional secondary agent included in the pharmaceutical formulation that achieves one or more therapeutic effects.

The effective amount, least effective amount, and/or therapeutically effective amount of the primary and optional secondary active agent described elsewhere herein contained in the pharmaceutical formulation can be any non-zero amount ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pg, ng, g, mg, or g or be any numerical value or subrange within any of these ranges.

In some embodiments, the effective amount, least effective amount, and/or therapeutically effective amount can be an effective concentration, least effective concentration, and/or therapeutically effective concentration, which can each be any non-zero amount ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pM, nM, M, mM, or M or be any numerical value or subrange within any of these ranges.

In other embodiments, the effective amount, least effective amount, and/or therapeutically effective amount of the primary and optional secondary active agent be any non-zero amount ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 IU or be any numerical value or subrange within any of these ranges.

In some embodiments, the primary and/or the optional secondary active agent present in the pharmaceutical formulation can be any non-zero amount ranging from about 0 to 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.9, to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the pharmaceutical formulation or be any numerical value or subrange within any of these ranges.

In some embodiments where a cell or cell population is present in the pharmaceutical formulation (e.g., as a primary and/or or secondary active agent), the effective amount of cells can be any amount ranging from about 1 or 2 cells to 1×10¹ cells/mL, 1×10²⁰ cells/mL or more, such as about 1×10¹ cells/mL, 1×10² cells/mL, 1×10³ cells/mL, 1×10⁴ cells/mL, 1×10⁵ cells/mL, 1×10⁶ cells/mL, 1×10⁷ cells/mL, 1×10⁸ cells/mL, 1×10⁹ cells/mL, 1×10¹⁰ cells/mL, 1×10¹¹ cells/mL, 1×10¹² cells/mL, 1×10¹³ cells/mL, 1×10¹⁴ cells/mL, 1×10¹⁵ cells/mL, 1×10¹⁶ cells/mL, 1×10¹⁷ cells/mL, 1×10¹⁸ cells/mL, 1×10¹⁹ cells/mL, to/or about 1×10²⁰ cells/mL or any numerical value or subrange within any of these ranges.

In some embodiments, the amount or effective amount, particularly where an infective particle is being delivered (e.g., a virus particle having the primary or secondary agent as a cargo), the effective amount of virus particles can be expressed as a titer (plaque forming units per unit of volume) or as a MOI (multiplicity of infection). In some embodiments, the effective amount can be about 1×10¹ particles per pL, nL, μL, mL, or L to 1×10²⁰/particles per pL, nL, μL, mL, or L or more, such as about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, to/or about 1×10²⁰ particles per pL, nL, μL, mL, or L. In some embodiments, the effective titer can be about 1×10¹ transforming units per pL, nL, μL, mL, or L to 1×10²⁰/transforming units per pL, nL, μL, mL, or L or more, such as about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, to/or about 1×10²⁰ transforming units per pL, nL, μL, mL, or L or any numerical value or subrange within these ranges. In some embodiments, the MOI of the pharmaceutical formulation can range from about 0.1 to 10 or more, such as 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10 or more or any numerical value or subrange within these ranges.

In some embodiments, the amount or effective amount of the one or more of the active agent(s) described herein contained in the pharmaceutical formulation can range from about 1 μg/kg to about 10 mg/kg based upon the bodyweight of the subject in need thereof or average bodyweight of the specific patient population to which the pharmaceutical formulation can be administered.

In embodiments where there is a secondary agent contained in the pharmaceutical formulation, the effective amount of the secondary active agent will vary depending on the secondary agent, the primary agent, the administration route, subject age, disease, stage of disease, among other things, which will be one of ordinary skill in the art.

When optionally present in the pharmaceutical formulation, the secondary active agent can be included in the pharmaceutical formulation or can exist as a stand-alone compound or pharmaceutical formulation that can be administered contemporaneously or sequentially with the compound, derivative thereof, or pharmaceutical formulation thereof.

In some embodiments, the effective amount of the secondary active agent, when optionally present, is any non-zero amount ranging from about 0 to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the total active agents present in the pharmaceutical formulation or any numerical value or subrange within these ranges. In additional embodiments, the effective amount of the secondary active agent is any non-zero amount ranging from about 0 to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the total pharmaceutical formulation or any numerical value or subrange within these ranges.

### Dosage Forms

In some embodiments, the pharmaceutical formulations described herein can be provided in a dosage form. The dosage form can be administered to a subject in need thereof. The dosage form can be effective generate specific concentration, such as an effective concentration, at a given site in the subject in need thereof. As used herein, “dose,” “unit dose,” or “dosage” can refer to physically discrete units suitable for use in a subject, each unit containing a predetermined quantity of the primary active agent, and optionally present secondary active ingredient, and/or a pharmaceutical formulation thereof calculated to produce the desired response or responses in association with its administration. In some embodiments, the given site is proximal to the administration site. In some embodiments, the given site is distal to the administration site. In some cases, the dosage form contains a greater amount of one or more of the active ingredients present in the pharmaceutical formulation than the final intended amount needed to reach a specific region or location within the subject to account for loss of the active components such as via first and second pass metabolism.

The dosage forms can be adapted for administration by any appropriate route. Appropriate routes include, but are not limited to, oral (including buccal or sublingual), rectal, intraocular, inhaled, intranasal, topical (including buccal, sublingual, or transdermal), vaginal, parenteral, subcutaneous, intramuscular, intravenous, internasal, and intradermal. Other appropriate routes are described elsewhere herein. Such formulations can be prepared by any method known in the art.

Dosage forms adapted for oral administration can discrete dosage units such as capsules, pellets or tablets, powders or granules, solutions, or suspensions in aqueous or non-aqueous liquids; edible foams or whips, or in oil-in-water liquid emulsions or water-in-oil liquid emulsions. In some embodiments, the pharmaceutical formulations adapted for oral administration also include one or more agents which flavor, preserve, color, or help disperse the pharmaceutical formulation. Dosage forms prepared for oral administration can also be in the form of a liquid solution that can be delivered as a foam, spray, or liquid solution. The oral dosage form can be administered to a subject in need thereof. Where appropriate, the dosage forms described herein can be microencapsulated.

The dosage form can also be prepared to prolong or sustain the release of any ingredient. In some embodiments, compounds, molecules, compositions, vectors, vector systems, cells, or a combination thereof described herein can be the ingredient whose release is delayed. In some embodiments the primary active agent is the ingredient whose release is delayed. In some embodiments, an optional secondary agent can be the ingredient whose release is delayed. Suitable methods for delaying the release of an ingredient include, but are not limited to, coating or embedding the ingredients in material in polymers, wax, gels, and the like. Delayed release dosage formulations can be prepared as described in standard references such as “Pharmaceutical dosage form tablets,” eds. Liberman et. al. (New York, Marcel Dekker, Inc., 1989), “Remington—The science and practice of pharmacy”, 20th ed., Lippincott Williams & Wilkins, Baltimore, MD, 2000, and “Pharmaceutical dosage forms and drug delivery systems”, 6th Edition, Ansel et al., (Media, PA: Williams and Wilkins, 1995). These references provide information on excipients, materials, equipment, and processes for preparing tablets and capsules and delayed release dosage forms of tablets and pellets, capsules, and granules. The delayed release can be anywhere from about an hour to about 3 months or more.

Examples of suitable coating materials include, but are not limited to, cellulose polymers such as cellulose acetate phthalate, hydroxypropyl cellulose, hydroxypropyl methylcellulose, hydroxypropyl methylcellulose phthalate, and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Coatings may be formed with a different ratio of water-soluble polymer, water insoluble polymers, and/or pH dependent polymers, with or without water insoluble/water soluble non-polymeric excipient, to produce the desired release profile. The coating is either performed on the dosage form (matrix or simple) which includes, but is not limited to, tablets (compressed with or without coated beads), capsules (with or without coated beads), beads, particle compositions, “ingredient as is” formulated as, but not limited to, suspension form or as a sprinkle dosage form.

Where appropriate, the dosage forms described herein can be a liposome. In these embodiments, primary active ingredient(s), and/or optional secondary active ingredient(s), and/or pharmaceutically acceptable salt thereof where appropriate are incorporated into a liposome. In embodiments where the dosage form is a liposome, the pharmaceutical formulation is thus a liposomal formulation. The liposomal formulation can be administered to a subject in need thereof.

Dosage forms adapted for topical administration can be formulated as ointments, creams, suspensions, lotions, powders, solutions, pastes, gels, sprays, aerosols, or oils. In some embodiments for treatments of the eye or other external tissues, for example the mouth or the skin, the pharmaceutical formulations are applied as a topical ointment or cream. When formulated in an ointment, a primary active ingredient, optional secondary active ingredient, and/or pharmaceutically acceptable salt thereof where appropriate can be formulated with a paraffinic or water-miscible ointment base. In other embodiments, the primary and/or secondary active ingredient can be formulated in a cream with an oil-in-water cream base or a water-in-oil base. Dosage forms adapted for topical administration in the mouth include lozenges, pastilles, and mouth washes.

Dosage forms adapted for nasal or inhalation administration include aerosols, solutions, suspension drops, gels, or dry powders. In some embodiments, a primary active ingredient, optional secondary active ingredient, and/or pharmaceutically acceptable salt thereof where appropriate can be in a dosage form adapted for inhalation is in a particle-size-reduced form that is obtained or obtainable by micronization. In some embodiments, the particle size of the size reduced (e.g., micronized) compound or salt or solvate thereof, is defined by a D₅₀ value of about 0.5 to about 10 microns as measured by an appropriate method known in the art. Dosage forms adapted for administration by inhalation also include particle dusts or mists. Suitable dosage forms wherein the carrier or excipient is a liquid for administration as a nasal spray or drops include aqueous or oil solutions/suspensions of an active (primary and/or secondary) ingredient, which may be generated by various types of metered dose pressurized aerosols, nebulizers, or insufflators. The nasal/inhalation formulations can be administered to a subject in need thereof.

In some embodiments, the dosage forms are aerosol formulations suitable for administration by inhalation. In some of these embodiments, the aerosol formulation contains a solution or fine suspension of a primary active ingredient, secondary active ingredient, and/or pharmaceutically acceptable salt thereof where appropriate and a pharmaceutically acceptable aqueous or non-aqueous solvent. Aerosol formulations can be presented in single or multi-dose quantities in sterile form in a sealed container. For some of these embodiments, the sealed container is a single dose or multi-dose nasal or an aerosol dispenser fitted with a metering valve (e.g., metered dose inhaler), which is intended for disposal once the contents of the container have been exhausted.

Where the aerosol dosage form is contained in an aerosol dispenser, the dispenser contains a suitable propellant under pressure, such as compressed air, carbon dioxide, or an organic propellant, including but not limited to a hydrofluorocarbon. The aerosol formulation dosage forms in other embodiments are contained in a pump-atomizer. The pressurized aerosol formulation can also contain a solution or a suspension of a primary active ingredient, optional secondary active ingredient, and/or pharmaceutically acceptable salt thereof. In further embodiments, the aerosol formulation also contains co-solvents and/or modifiers incorporated to improve, for example, the stability and/or taste and/or fine particle mass characteristics (amount and/or profile) of the formulation. Administration of the aerosol formulation can be once daily or several times daily, for example 2, 3, 4, or 8 times daily, in which 1, 2, 3 or more doses are delivered each time. The aerosol formulations can be administered to a subject in need thereof.

For some dosage forms suitable and/or adapted for inhaled administration, the pharmaceutical formulation is a dry powder inhalable-formulations. In addition to a primary active agent, optional secondary active ingredient, and/or pharmaceutically acceptable salt thereof where appropriate, such a dosage form can contain a powder base such as lactose, glucose, trehalose, mannitol, and/or starch. In some of these embodiments, a primary active agent, secondary active ingredient, and/or pharmaceutically acceptable salt thereof where appropriate is in a particle-size reduced form. In further embodiments, a performance modifier, such as L-leucine or another amino acid, cellobiose octaacetate, and/or metals salts of stearic acid, such as magnesium or calcium stearate. In some embodiments, the aerosol formulations are arranged so that each metered dose of aerosol contains a predetermined amount of an active ingredient, such as the one or more of the compositions, compounds, vector(s), molecules, cells, and combinations thereof described herein.

Dosage forms adapted for vaginal administration can be presented as pessaries, tampons, creams, gels, pastes, foams, or spray formulations. Dosage forms adapted for rectal administration include suppositories or enemas. The vaginal formulations can be administered to a subject in need thereof.

Dosage forms adapted for parenteral administration and/or adapted for injection can include aqueous and/or non-aqueous sterile injection solutions, which can contain antioxidants, buffers, bacteriostats, solutes that render the composition isotonic with the blood of the subject, and aqueous and non-aqueous sterile suspensions, which can include suspending agents and thickening agents. The dosage forms adapted for parenteral administration can be presented in a single-unit dose or multi-unit dose containers, including but not limited to sealed ampoules or vials. The doses can be lyophilized and re-suspended in a sterile carrier to reconstitute the dose prior to administration. Extemporaneous injection solutions and suspensions can be prepared in some embodiments, from sterile powders, granules, and tablets. The parenteral formulations can be administered to a subject in need thereof.

For some embodiments, the dosage form contains a predetermined amount of a primary active agent, secondary active ingredient, and/or pharmaceutically acceptable salt thereof where appropriate per unit dose. In an embodiment, the predetermined amount of primary active agent, secondary active ingredient, and/or pharmaceutically acceptable salt thereof where appropriate can be an effective amount, a least effect amount, and/or a therapeutically effective amount. In other embodiments, the predetermined amount of a primary active agent, secondary active agent, and/or pharmaceutically acceptable salt thereof where appropriate, can be an appropriate fraction of the effective amount of the active ingredient.

### Co-Therapies and Combination Therapies

In some embodiments, the pharmaceutical formulation(s) described herein are part of a combination treatment or combination therapy. The combination treatment can include the pharmaceutical formulation described herein and an additional treatment modality. The additional treatment modality can be a chemotherapeutic, a biological therapeutic, surgery, radiation, diet modulation, environmental modulation, a physical activity modulation, and combinations thereof.

In some embodiments, the co-therapy or combination therapy can additionally include but not limited to, polynucleotides, amino acids, peptides, polypeptides, antibodies, aptamers, ribozymes, hormones, immunomodulators, antipyretics, anxiolytics, antipsychotics, analgesics, antispasmodics, anti-inflammatories, anti-histamines, anti-infectives, chemotherapeutics, and combinations thereof.

### Administration of the Pharmaceutical Formulations

The pharmaceutical formulations or dosage forms thereof described herein can be administered one or more times hourly, daily, monthly, or yearly (e.g., 1, 2, 3, 4, 5, 6, 7, 8, 9, 10, 11, 12, 13, 14, 15, 16, 17, 18, 19, 20, or more times hourly, daily, monthly, or yearly). In some embodiments, the pharmaceutical formulations or dosage forms thereof described herein can be administered continuously over a period of time ranging from minutes to hours to days. Devices and dosages forms are known in the art and described herein that are effective to provide continuous administration of the pharmaceutical formulations described herein. In some embodiments, the first one or a few initial amount(s) administered can be a higher dose than subsequent doses. This is typically referred to in the art as a loading dose or doses and a maintenance dose, respectively. In some embodiments, the pharmaceutical formulations can be administered such that the doses over time are tapered (increased or decreased) overtime so as to wean a subject gradually off of a pharmaceutical formulation or gradually introduce a subject to the pharmaceutical formulation.

As previously discussed, the pharmaceutical formulation can contain a predetermined amount of a primary active agent, secondary active agent, and/or pharmaceutically acceptable salt thereof where appropriate. In some of these embodiments, the predetermined amount can be an appropriate fraction of the effective amount of the active ingredient. Such unit doses may therefore be administered once or more than once a day, month, or year (e.g., 1, 2, 3, 4, 5, 6, or more times per day, month, or year). Such pharmaceutical formulations may be prepared by any of the methods well known in the art.

Where co-therapies or multiple pharmaceutical formulations are to be delivered to a subject, the different therapies or formulations can be administered sequentially or simultaneously. Sequential administration is administration where an appreciable amount of time occurs between administrations, such as more than about 15, 20, 30, 45, 60 minutes or more. The time between administrations in sequential administration can be on the order of hours, days, months, or even years, depending on the active agent present in each administration. Simultaneous administration refers to administration of two or more formulations at the same time or substantially at the same time (e.g., within seconds or just a few minutes apart), where the intent is that the formulations be administered together at the same time.

### Kits

Described herein are certain example embodiments herein are kits comprising a nudiviral nucleic acid of the present description; an engineered polynucleotide of the present description; a vector or vector system of the present description; a cell or cell population as in the present description; or a formulation of the present description, or any combination thereof.

Any of the compounds, compositions, formulations, particles, cells, described herein or a combination thereof can be presented as a combination kit. As used herein, the terms “combination kit” or “kit of parts” refers to the compounds, compositions, formulations, particles, cells and any additional components that are used to package, sell, market, deliver, and/or administer or otherwise use the combination of elements or a single element, such as the active ingredient, contained therein. Such additional components include, but are not limited to, packaging, syringes, blister packages, bottles, and the like. When one or more of the compounds, compositions, formulations, particles, cells, described herein or a combination thereof (e.g., agents) contained in the kit are administered simultaneously, the combination kit can contain the active agents in a single formulation, such as a pharmaceutical formulation, (e.g., a tablet) or in separate formulations. When the compounds, compositions, formulations, particles, and cells described herein or a combination thereof and/or kit components are not administered simultaneously, the combination kit can contain each agent or other component in separate pharmaceutical formulations. The kits can contain one or more additional reagents, buffers, preservatives, and/or the like that can be used with any one or more of the compounds, compositions, formulations, particles, and cells of the present disclosure. The separate kit components can be contained in a single package or in separate packages within the kit.

In some embodiments, the combination kit also includes instructions printed on or otherwise contained in a tangible medium of expression. The instructions can provide information regarding the content of the compounds, compositions, formulations, particles, cells, described herein or a combination thereof contained therein, safety information regarding the content of the compounds, compositions, formulations (e.g., pharmaceutical formulations, vaccines, and/or the like), particles, and cells described herein or a combination thereof contained therein, information regarding the dosages, indications for use, and/or recommended treatment regimen(s) for the compound(s) and/or pharmaceutical formulations contained therein. In some embodiments, the instructions can provide directions for administering the compounds, compositions, formulations, particles, and cells described herein or a combination thereof to a subject in need thereof.

### Methods

Described in certain example embodiments herein are methods comprising expressing an engineered polynucleotide of the present description or a vector or vector system as in any one of the present description in vitro, in vivo, or ex vivo.

Described in certain example embodiments herein are methods of expressing an engineered nucleic acid, the method comprising placing an engineered polynucleotide of the present description or a vector or vector system of the present description under condition(s) and/or environment(s) such that the non-polyhedrin polynucleotide is transcribed and optionally translated. In certain example embodiments, wherein expression occurs in vitro, in vivo, or ex vivo. In certain example embodiments, the method further comprises operatively coupling the non-polyhedrin encoding nucleic acid to a nudiviral nucleic acid of the present description.

Delivery methods, techniques, and devices for delivering and/or expressing a polynucleotide via a nudiviral nucleic acid are described in greater detail elsewhere herein.

Further embodiments are illustrated in the following Examples which are given for illustrative purposes only and are not intended to limit the scope of the invention.

### EXAMPLES

Now having described the embodiments of the present disclosure, in general, the following Examples describe some additional embodiments of the present disclosure. While embodiments of the present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure. The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in °C, and pressure is at or near atmospheric. Standard temperature and pressure are defined as 20 °C and 1 atmosphere.

### Example 1

### Introduction

Commercial shrimp farming started in Asia in 1970 and has grown steadily, reaching an estimated production of 4,000 metric tons in 2019 to meet a growing market demand [1]. With the expansion of shrimp aquaculture, infectious diseases became more problematic and the list of identified shrimp diseases continues to grow in parallel with the growth global shrimp industry [2, 3].

Tetrahedral baculovirus, commonly known as Baculovirus penaei (BP) or Penaeus vannamei singly enveloped nuclear polyhedrosis virus (PvSNPV) was first reported in the hepatopancreas of pink shrimp, Penaeus duorarum, in 1974 in Gulf of Mexico [4, 5] Later, PvSNPV was reported to cause infections in most penaeid shrimp and during all life stages [6]. The World Organization for Animal Health (OIE, Paris, France) listed PvSNPV as a notifiable crustacean viral pathogen until 2009. In 2009, PvSNPV was delisted because the economic impacts caused by PvSNPV were not as significant as in the past, due to the adoption of simple disease management and biosecurity tools such as washing eggs with iodine containing water, separating stronger post-larvae from those that are relatively weaker, and quarantining of incoming PLs. Morphologically, the virions are rod-shaped, enveloped particles measuring 312 to 320 nm in length and 75 to 87 nm in diameter. The nucleocapsids, 306 to 312 nm in length and 62 to 68 nm in diameter, with a crosshatched surface arranged in a helical pattern and a trilaminar structure capping both extremities in the virion [7]. The viral particles are present both free and within pyramidal-shaped polyhedral inclusion bodies in the nuclei of hepatopancreatic and midgut cells [5]. The virus was classified as baculovirus because of the morphological similarity of the mature virions to viruses belonging to the family Baculoviridae. Viruses in the Baculoviridae are the most common insect pathogenic viruses with 20 known groups subdivided into 12 viral families [8, 9]. In addition, baculoviruses are also well known as vectors for transduction of mammalian cells, gene expression vectors, and pest control treatments [10-12]. Transmission electron micrograph (TEM) analyses revealed that baculoviruses had rod shaped nucleocapsids surrounded by an envelope [8, 13]. Interestingly, in the 1974 paper authored by Couch and published in the journal Nature, the author reported that there are differences in lattice line to line spacing between PvSNPV and other baculoviruses infecting to insects, suggesting PvSNPV may not belong to Baculoviridae family.

The pathobiology of PvSNPV has been investigated in only a few studies. For instance, Couch hypothesized that viral particle assembly and occlusion into polyhedral bodies occurs within 24 hour post infection [14]. In 1988, the first successful PvSNPV experimental challenge trail was done by Overstreet and colleagues who infected P. vannamei post-larvae 3 or mysis using Brachionus plicatilis and Artemia salina as the viral delivery vehicle [15]. Additionally, it was reported the non-occluded viral particles could provoke infection in P. vannamei post-larvae 10 days post-infection [16]. While the pathobiology of PvSNPV was studied [5, 14, 16], during the ensuing 45 years PvSNPV genome has not been sequenced. Before this study, the largest sequence of PvSNPV deposited in NCBI was less than 2 kbp region providing the polyhedrin gene sequence. Due to this lack of the full genome sequence, taxonomy of the virus remained unclear, and the molecular basis of viral pathogenicity remained obscure.

In this study, next generation sequencing (NGS) was employed to determine the genome sequence of PvSNPV from P. vannamei broodstock that tested positive for the virus. The viral genome was then annotated and the phylogenetic relationship of PvSNPV to other viruses in the families, Baculoviridae and Nudiviridae was evaluated. Based on the genomic features and phylogenetic relations identified in this study, Applicant proposes the renaming of BP/PvSNPV as Penaeus vannamei nudivirus (PvNV) and suggests its reassignment to the family Nudiviridae instead of Baculoviridae

### Results

Detection of PvSNPV in Broodstock and Experimental Challenge of Penaeus vannamei Post-Larvae with PvSNPV

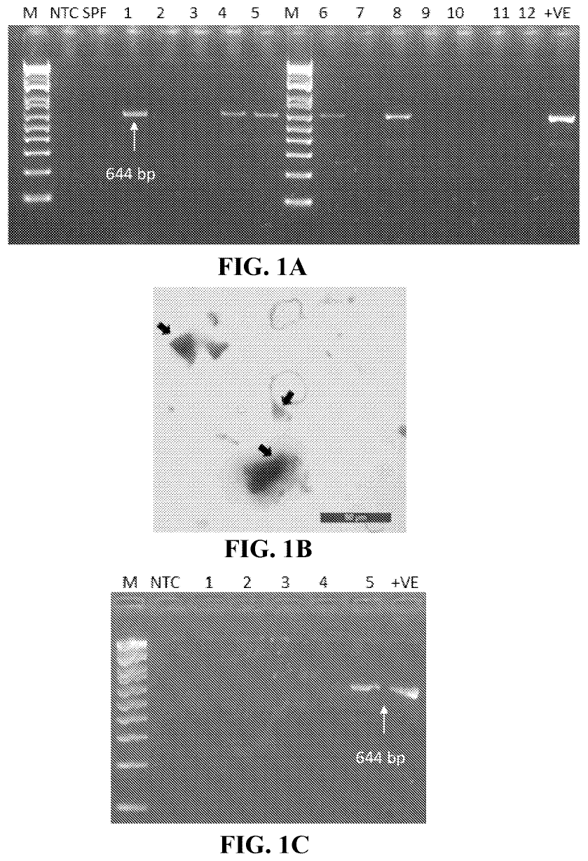

Penaeus vannamei broodstock were maintained in a quarantine facility for a nucleus breeding program in the University of Arizona, Aquaculture Pathology Laboratory. As a part of routine health check, broodstock were screened for a panel of enteric and systemic viral, bacterial, and fungal pathogens. PvSNV was one of the pathogens that was tested for by a conventional PCR method using fecal DNA samples. Five out of twelve broodstock tested positive for PvSNPV (FIG. 1A-1B). However, these broodstock tested negative for the remaining pathogens.

The PvSNPV-positive broodstock were sacrificed and hepatopancreas tissue (HP) taken to conduct an experimental challenge using SPF P. vannamei post-larvae, as described in the materials and methods section. A wet mount from HP isolated from experimentally challenged shrimp displayed “pyramid” like occlusion bodies in the hepatopancreatic tubule epithelial cells and in the lumen of the tubule. (FIG. 1A-1B). Subsequently, conventional PCR was performed to detect PvSNPV in the post-larvae. One out of five shrimp from the challenged tanks tested positive for PvSNPV (FIG. 1C).

### De Novo Assembly of PvSNPV

Illumina sequencing generated 13,123,924 unpaired reads. The number of paired reads after adapter and low-quality trimming were 10,833,122 paired reads. A total of 9,408,536 paired reads was used to conduct de novo assembly after P. vannamei genome and duplicated read removal. De novo assembly results showed that 7,224,141 reads were assembled to produce 1,659,960 contigs in which 8,521 contigs were longer than 1000 bp in length with a N50=1900 bp. The largest contig was 119,883 bp in length and it showed similarity to the PvSNPV partial sequence available in the GenBank database by BLASTN (highly similar sequences) search. PvSNPV contigs had a 234.4× of mean coverage and 33.4% of GC content (FIG. 2). The PvSNPV genome was deposited in GenBlank with accession number as OM066077.

### Characterization of PvSNPV Genome

### Open Reading Frame (ORF) Prediction

Based on the results from three programs (Prokka, GeneMarks, fgeneV0), 100 ORFs ranging from 240 to 3800 bp in length were annotated (Table 1) and mapped on the genome (FIG. 2). In addition, the number of annotated ORFs were equal between forward and reverse strands (50 OR6s in forward strand; 50 ORFs in reverse strand). The gene density was approximately 0.8 gene per kbp

TABLE 1
Identified Open reading frame (ORF) in the PvSNPV genome. The start and stop positions were shown as well
as the length (in nucleotide (nt) and amino acid (aa)), the strand (“+” forward strand; “−”
reverse strand). The best hits obtained using BlastP (protein-protein blast) using non-redundant protein
sequences (nr) database on NCBI (E-value < 0.001). Additional information was obtained from orthologous analysis.

| | | | Length | | | Best hit (name/description/ | | E- | Additional |
|---|---|---|---|---|---|---|---|---|---|
| ORF | Start | Stop | nt | aa | Strand | accession number) | % id | value | annotation |
| 1 | 1 | 3306 | 3306 | 1101 | + | DNA polymerase [Penaeus | 59.89 | 0.00 | |
| | | | | | | monodon nudivirus] | | | |
| | | | | | | (YP_009051843.1) | | | |
| 2 | 3347 | 4561 | 1215 | 404 | + | methyltransferase [Penaeus | 43.92 | 6.00 × 10⁻¹⁰⁶ | |
| | | | | | | monodon nudivirus] | | | |
| | | | | | | (YP_009051844.1) | | | |
| 3 | 4776 | 5447 | 672 | 223 | − | Ac92-like protein [Penaeus | 53.43 | 1.00 × 10⁻⁷² | |
| | | | | | | monodon nudivirus] | | | |
| | | | | | | (YP_009051846.1) | | | |
| 4 | 5425 | 7467 | 2043 | 680 | + | Vp91 [Penaeus monodon nudivirus] | 48.15 | 0.00 | |
| | | | | | | (YP_009051847.1) | | | |
| 5 | 7603 | 8925 | 1323 | 440 | − | ODV-E56 [Penaeus monodon | 57.01 | 5.00 × 10⁻¹⁷⁷ | |
| | | | | | | nudivirus] (YP_009051848.1) | | | |
| 6 | 9005 | 9484 | 480 | 159 | − | hypothetical protein PmNV-012 | 40.25 | 1.00 × 10⁻³³ | |
| | | | | | | [Penaeus monodon nudivirus] | | | |
| | | | | | | (YP_009051850.1) | | | |
| 7 | 9494 | 10777 | 1284 | 427 | + | p47 [Penaeus monodon nudivirus] | 50.83 | 4.00 × 10⁻¹⁵¹ | |
| | | | | | | (YP_009051852.1) | | | |
| 8 | 10782 | 11912 | 1131 | 376 | + | PIF-2 [Penaeus monodon nudivirus] | 61.6 | 3.00 × 10⁻¹⁷¹ | |
| | | | | | | (YP_009051853.1) | | | |
| 9 | 11955 | 12746 | 792 | 263 | − | HZV-115 like protein [Penaeus | 32.77 | 2.00 × 10⁻³⁴ | |
| | | | | | | monodon nudivirus] | | | |
| | | | | | | (YP_009051855.1) | | | |
| 10 | 12834 | 14183 | 1350 | 449 | − | hypothetical protein PmNV-019 | 34.19 | 8.00 × 10⁻⁶⁴ | |
| | | | | | | [Penaeus monodon nudivirus] | | | |
| | | | | | | (YP_009051856.1) | | | |
| 11 | 14207 | 14587 | 381 | 126 | + | hypothetical protein PmNV-019 | 45.67 | 5.00 × 10⁻²⁵ | |
| | | | | | | [Penaeus monodon nudivirus] | | | |
| | | | | | | (YP_009051857.1) | | | |
| 12 | 14545 | 15813 | 1269 | 422 | + | hypothetical protein PmNV-020 | 45.52 | 2.00 × 10⁻¹¹⁴ | FEN-1 |
| | | | | | | [Penaeus monodon nudivirus] | | | |
| | | | | | | (YP_009051858.1) | | | |
| 13 | 15800 | 16273 | 474 | 157 | − | hypothetical protein PmNV-021 | 37.34 | 2.00 × 10⁻²⁷ | |
| | | | | | | [Penaeus monodon nudivirus] | | | |
| | | | | | | (YP_009051859.1) | | | |
| 14 | 16282 | 17202 | 921 | 306 | − | hypothetical protein PmNV-022 | 58.58 | 2.00 × 10⁻¹³⁰ | |
| | | | | | | [Penaeus monodon nudivirus] | | | |
| | | | | | | (YP_009051860.1) | | | |
| 15 | 17320 | 20415 | 3096 | 1031 | − | PmNV-LEF8 [Penaeus monodon | 57.23 | 0.00 | |
| | | | | | | nudivirus] (YP_009051861.1) | | | |
| 16 | 20595 | 21818 | 1224 | 407 | − | hypothetical protein PmNV-P51 | 53.71 | 7.00 × 10⁻¹⁴⁰ | |
| | | | | | | [Penaeus monodon nudivirus] | | | |
| | | | | | | (YP_009051862.1) | | | |
| 17 | 22084 | 25659 | 3576 | 1191 | + | hypothetical protein PmNV-025 | 42.74 | 1.00 × 10⁻¹⁴⁶ | |
| | | | | | | [Penaeus monodon nudivirus] | | | |
| | | | | | | (YP_009051863.1) | | | |
| 18 | 25813 | 27027 | 1215 | 404 | − | — | | | |
| 19 | 29758 | 30054 | 297 | 98 | + | — | | | |
| 20 | 30117 | 32012 | 1896 | 631 | − | ODV-E66 [Penaeus monodon | 54.76 | 0.00 | |
| | | | | | | nudivirus] (YP_009051874.1) | | | |
| 21 | 32538 | 33569 | 1032 | 343 | − | — | | | |
| 22 | 33745 | 33984 | 240 | 79 | − | — | | | |
| 23 | 34049 | 34978 | 930 | 309 | + | guanosine monophosphate kinase | 47.78 | 4.00 × 10⁻⁸⁸ | Tk2 |
| | | | | | | [Penaeus monodon nudivirus] | | | |
| | | | | | | (YP_009051876.1) | | | |
| 24 | 35027 | 36598 | 1572 | 523 | + | PIF-1 [Penaeus monodon nudivirus] | 52.85 | 0.00 | |
| | | | | | | (YP_009051877.1) | | | |
| 25 | 36623 | 37264 | 642 | 213 | − | hypothetical protein PmNV_040 | 38.52 | 4.00 × 10⁻¹⁵ | |
| | | | | | | [Penaeus monodon nudivirus] | | | |
| | | | | | | (YP_009051878.1) | | | |
| 26 | 37267 | 39576 | 2310 | 769 | + | hypothetical protein PmNV_042 | 44.53 | 6.00 × 10⁻¹⁵³ | |
| | | | | | | [Penaeus monodon nudivirus] | | | |
| | | | | | | (YP_009051880.1) | | | |
| 27 | 39567 | 40367 | 801 | 266 | − | hypothetical protein PmNV_043 | 50.61 | 2.00 × 10⁻⁸⁶ | |
| | | | | | | [Penaeus monodon nudivirus] | | | |
| | | | | | | (YP_009051881.1) | | | |
| 28 | 40386 | 41126 | 741 | 246 | − | hypothetical protein PmNV_044 | 27.78 | 2.00 × 10⁻¹⁷ | |
| | | | | | | [Penaeus monodon nudivirus] | | | |
| | | | | | | (YP_009051882.1) | | | |
| 29 | 41126 | 41890 | 765 | 254 | + | PmV-like protein [Penaeus monodon | 56.68 | 3.00 × 10⁻⁹⁶ | |
| | | | | | | nudivirus] (YP_009051883.1) | | | |
| 30 | 41938 | 43074 | 1137 | 378 | + | p-loop NTPase [Penaeus monodon | 48.22 | 7.00 × 10⁻¹⁰² | Tk3 |
| | | | | | | nudivirus] (YP_009051884.1) | | | |
| 31 | 43142 | 44062 | 921 | 306 | + | hypothetical protein PmNV_047 | 45.54 | 1.00 × 10⁻⁸² | |
| | | | | | | [Penaeus monodon nudivirus] | | | |
| | | | | | | (YP_009051885.1) | | | |
| 32 | 44067 | 45863 | 1797 | 598 | − | hypothetical protein PmNV_048 | 49.75 | 0.00 | |
| | | | | | | [Penaeus monodon nudivirus] | | | |
| | | | | | | (YP_009051886.1) | | | |
| 33 | 45842 | 46747 | 906 | 301 | + | hypothetical protein | 25.1 | 1.92 × 10⁻¹⁰⁹ | |
| | | | | | | CFAM422_012008 [Trichoderma | | | |
| | | | | | | lentiforme] (KAF3057811.1) | | | |
| 34 | 46907 | 47923 | 1017 | 338 | + | hypothetical protein LY90DRAFT_{—} | 29 | 7.60 × 10⁻⁰⁷ | |
| | | | | | | 74522 [Neocallimastix californiae] | | | |
| | | | | | | (ORY32362) | | | |
| 35 | 47925 | 48248 | 324 | 107 | + | hypothetical protein [Penaeus | 42.72 | 2.00 × 10⁻¹⁵ | |
| | | | | | | mondon nudivirus] (ABX44699) | | | |
| 36 | 48455 | 48943 | 489 | 162 | + | hypothetical protein [Penaeus | 35.43 | 4.00 × 10⁻¹⁶ | LEF-5 |
| | | | | | | monodon nucleopolyhedrovirus] | | | |
| | | | | | | (ABX44700.1) | | | |
| 37 | 49005 | 49268 | 264 | 87 | + | — | | | |
| 38 | 49478 | 50137 | 660 | 219 | + | hypothetical protein PmNV_054 | 45.91 | 7.00 × 10⁻⁵⁵ | |
| | | | | | | [Penaeus monodon nudivirus] | | | |
| | | | | | | (YP_009051892.1) | | | |
| 39 | 50178 | 51089 | 912 | 303 | − | integrase [Penaeus monodon | 61.06 | 1.00 × 10⁻¹³⁸ | |
| | | | | | | nudivirus] (YP_009051893.1) | | | |
| 40 | 51082 | 51951 | 870 | 289 | − | hypothetical protein [Penaeus | 52.1 | 1.68 × 10⁻⁸⁷ | Vlf1 |
| | | | | | | mondon nudivirus] (ABX44703) | | | |
| 41 | 51941 | 52273 | 333 | 110 | − | — | | | |
| 42 | 52263 | 54011 | 1749 | 582 | − | LEF-9 [Penaeus monodon nudivirus] | 61.76 | 0.00E+00 | |
| | | | | | | (YP_009051896.1) | | | |
| 43 | 54029 | 54874 | 846 | 281 | + | 38K protein [Penaeus monodon | 50 | 3.00 × 10⁻⁸⁵ | |
| | | | | | | nudivirus] (YP_009051897.1) | | | |
| 44 | 54883 | 55167 | 285 | 94 | − | hypothetical protein PmNV_060 | 52.13 | 2.00 × 10⁻²³ | |
| | | | | | | [Penaeus monodon nudivirus] | | | |
| | | | | | | (YP_009051898.1) | | | |
| 45 | 55169 | 55861 | 693 | 230 | − | hypothetical protein PmNV_061 | 54.74 | 7.00 × 10⁻⁸¹ | |
| | | | | | | [Penaeus monodon nudivirus] | | | |
| | | | | | | (YP_009051899.1) | | | |
| 46 | 55863 | 56276 | 414 | 137 | − | hypothetical protein PmNV_062 | 27.94 | 2.00 × 10⁻⁰⁷ | |
| | | | | | | [Penaeus monodon nudivirus] | | | |
| | | | | | | (YP_009051900.1) | | | |
| 47 | 56289 | 56681 | 393 | 130 | − | hypothetical protein PmNV_063 | 31.58 | 2.00 × 10⁻¹⁰ | |
| | | | | | | [Penaeus monodon nudivirus] | | | |
| | | | | | | (YP_009051901.1) | | | |
| 48 | 56821 | 57174 | 354 | 117 | + | — | | | |
| 49 | 57238 | 58557 | 1320 | 439 | + | p-loop NTPase [Penaeus monodon | 49.55 | 9.00 × 10⁻¹⁵⁹ | Tk1 |
| | | | | | | nudivirus] (YP_009051903.1) | | | |
| 50 | 58623 | 59804 | 1182 | 393 | − | hypothetical protein PmNV_066 | 45.34 | 2.00 × 10⁻¹⁰⁹ | |
| | | | | | | [Penaeus monodon nudivirus] | | | |
| | | | | | | (YP_009051904.1) | | | |
| 51 | 59876 | 60403 | 528 | 175 | − | hypothetical protein PmNV_067 | 32.77 | 4.00 × 10⁻¹⁹ | |
| | | | | | | [Penaeus monodon nudivirus] | | | |
| | | | | | | (YP_009051905.1) | | | |
| 52 | 60555 | 61166 | 612 | 203 | − | hypothetical protein PmNV_068 | 40.78 | 7.00 × 10⁻³² | |
| | | | | | | [Penaeus monodon nudivirus] | | | |
| | | | | | | (YP_009051906.1) | | | |
| 53 | 61196 | 62176 | 981 | 326 | + | hypothetical protein PmNV_069 | 54.68 | 3.00 × 10⁻¹⁴ | |
| | | | | | | [Penaeus monodon nudivirus] | | | |
| | | | | | | (YP_009051907.1) | | | |
| 54 | 62154 | 62750 | 597 | 198 | − | hypothetical protein PmNV_070 | 48.73 | 3.00 × 10⁻⁴⁹ | |
| | | | | | | [Penaeus monodon nudivirus] | | | |
| | | | | | | (YP_009051908.1) | | | |
| 55 | 62936 | 64315 | 1380 | 459 | + | hypothetical protein PmNV_071 | 31.36 | 4.00 × 10⁻⁵³ | |
| | | | | | | [Penaeus monodon nudivirus] | | | |
| | | | | | | (YP_009051909.1) | | | |
| 56 | 64350 | 66431 | 2082 | 693 | + | P74 [Penaeus monodon nudivirus] | 57.56 | 0.00 | |
| | | | | | | (YP_009051910.1) | | | |
| 57 | 66525 | 67217 | 693 | 230 | + | ephrin type-B receptor 4-like | 52.46 | 9.00 × 10⁻⁰⁷ | |
| | | | | | | [Poecilia reticulata] | | | |
| | | | | | | (XP_017158981.1) | | | |
| 58 | 67159 | 67769 | 611 | 169 | + | hypothetical protein PmNV_075 | 39.39 | 5.00 × 10⁻²⁷ | |
| | | | | | | [Penaeus monodon nudivirus] | | | |
| | | | | | | (YP_009051913.1) | | | |
| 59 | 67852 | 69828 | 1977 | 658 | − | helicase 2 [Penaeus monodon | 53.62 | 0.00 | |
| | | | | | | nudivirus] (YP_009051914.1) | | | |
| 60 | 69873 | 70754 | 882 | 293 | + | hypothetical protein PmNV_077 | 27.97 | 2.00 × 10⁻¹⁹ | |
| | | | | | | [Penaeus monodon nudivirus] | | | |
| | | | | | | (YP_009051915.1) | | | |
| 61 | 70735 | 71361 | 627 | 208 | − | hypothetical protein PmNV_078 | 51.2 | 2.00 × 10⁻⁵³ | |
| | | | | | | [Penaeus monodon nudivirus] | | | |
| | | | | | | (YP_009051916.1) | | | |
| 62 | 71258 | 73339 | 2082 | 693 | + | helicase 2 [Penaeus monodon | 49.86 | 0.00 | |
| | | | | | | nudivirus] (YP_009051917.1) | | | |
| 63 | 73332 | 75878 | 2547 | 848 | + | hypothetical protein PmNV_080 | 28.86 | 1.00 × 10⁻⁵⁹ | |
| | | | | | | [Penaeus monodon nudivirus] | | | |
| | | | | | | (YP_009051918.1) | | | |
| 64 | 75880 | 76827 | 948 | 315 | + | hypothetical protein PmNV_082 | 45.34 | 3.00 × 10⁻⁸⁰ | |
| | | | | | | [Penaeus monodon nudivirus] | | | |
| | | | | | | (YP_009051920.1) | | | |
| 65 | 76818 | 77576 | 759 | 252 | − | hypothetical protein PmNV_083 | 26.67 | 1.10 × 10⁻¹² | |
| | | | | | | [Penaeus monodon nudivirus] | | | |
| | | | | | | (YP_009051921.1) | | | |
| 66 | 77573 | 79060 | 1488 | 495 | + | hypothetical protein PmNV_084 | 34.72 | 2.00 × 10⁻⁷⁶ | |
| | | | | | | [Penaeus monodon nudivirus] | | | |
| | | | | | | (YP_009051922.1) | | | |
| 67 | 79069 | 79479 | 411 | 136 | + | hypothetical protein PmNV_085 | 42.54 | 8.00 × 10⁻²⁸ | |
| | | | | | | [Penaeus monodon nudivirus] | | | |
| | | | | | | (YP_009051923.1) | | | |
| 68 | 79460 | 79963 | 504 | 167 | − | Ac81-like protein [Penaeus | 63.4 | 8.00 × 10⁻⁶⁷ | |
| | | | | | | monodon nudivirus] | | | |
| | | | | | | (YP_009051924.1) | | | |
| 69 | 79960 | 81879 | 1920 | 639 | + | hypothetical protein PmNV_087 | 41.6 | 2.00 × 10⁻¹⁵⁶ | |
| | | | | | | [Penaeus monodon nudivirus] | | | |
| | | | | | | (YP_009051925.1) | | | |
| 70 | 81876 | 82307 | 432 | 143 | + | PIF-6 [Carcinus maenas nudivirus] | 47.55 | 9.00 × 10⁻⁴³ | |
| | | | | | | (UBZ25668.1) | | | |
| 71 | 82415 | 83689 | 1275 | 424 | − | hypothetical protein PmNV_089 | 45.54 | 3.00 × 10⁻¹⁰⁹ | |
| | | | | | | [Penaeus monodon nudivirus] | | | |
| | | | | | | (YP_009051927.1) | | | |
| 72 | 83759 | 84469 | 711 | 236 | + | VLF-1 [Penaeus monodon | 56.17 | 1.00 × 10⁻⁷⁹ | |
| | | | | | | nudivirus] (YP_009051928.1) | | | |
| 73 | 84471 | 85949 | 1479 | 492 | + | LEF-4 [Penaeus monodon nudivirus] | 48.69 | 4.00 × 10⁻¹⁴⁷ | |
| | | | | | | (YP_009051929.1) | | | |
| 74 | 85964 | 86275 | 312 | 103 | − | hypothetical protein KM727_gp82 | 36.71 | 8.00 × 10⁻⁰⁴ | |
| | | | | | | [Homarus gammarus nudivirus] | | | |
| 75 | 86326 | 86922 | 597 | 198 | − | PIF-3 [Carcinus maenas nudivirus] | 51.03 | 2.00 × 10⁻⁶⁵ | |
| | | | | | | (UBZ25673.1) | | | |
| 76 | 86901 | 90710 | 3810 | 126 | − | helicase [Penaeus monodon | 52.54 | 0.00 | |
| | | | | 9 | | nudivirus] (YP_009051932.1) | | | |
| 77 | 90394 | 91458 | 1065 | 354 | − | ODV-E28 [Penaeus monodon | 51.5 | 8.00 × 10⁻⁷⁰ | |
| | | | | | | nudivirus] (YP_009051934.1) | | | |
| 78 | 91382 | 92185 | 804 | 267 | + | hypothetical protein PmNV_097 | 51 | 2.00 × 10⁻⁷⁹ | |
| | | | | | | [Penaeus monodon nudivirus] | | | |
| | | | | | | (YP_009051935.1) | | | |
| 79 | 92175 | 92999 | 825 | 274 | + | Esterase [Penaeus monodon | 43.12 | 1.00 × 10⁻⁶³ | |
| | | | | | | nudivirus] (YP_009051936.1) | | | |
| 80 | 93003 | 94232 | 1230 | 409 | + | hypothetical protein PmNV_099 | 51.55 | 2.00 × 10⁻⁵⁴ | GbNV-gb67- |
| | | | | | | [Penaeus monodon nudivirus] | | | like protein |
| | | | | | | (YP_009051937.1) | | | |
| 81 | 94310 | 94618 | 309 | 102 | + | 11K virion structural protein | 59.79 | 2.00 × 10⁻³⁵ | |
| | | | | | | [Penaeus monodon nudivirus] | | | |
| | | | | | | (YP_009051938.1) | | | |
| 82 | 94739 | 95095 | 357 | 118 | + | hypothetical protein PmNV_101 | | | |
| | | | | | | [Penaeus monodon nudivirus] | | | |
| | | | | | | (YP_009051939.1) | | | |
| 83 | 95082 | 95879 | 798 | 265 | − | hypothetical protein PmNV_102 | 56.23 | 4.00 × 10⁻¹⁰⁰ | |
| | | | | | | [Penaeus monodon nudivirus] | | | |
| | | | | | | (YP_009051940.1) | | | |
| 84 | 95867 | 97246 | 1380 | 459 | − | none | | | |
| 85 | 98733 | 99446 | 714 | 237 | + | hypothetical protein PmNV_107 | 71.55 | 4.00 × 10⁻¹²³ | |
| | | | | | | [Penaeus monodon nudivirus] | | | |
| | | | | | | YP_009051945.1 | | | |
| 86 | 99573 | 100256 | 684 | 227 | + | hypothetical protein PmNV_108 | 52.89 | 2.00 × 10⁻⁸² | |
| | | | | | | [Penaeus monodon nudivirus] | | | |
| | | | | | | (YP_009051946.1) | | | |
| 87 | 100348 | 100887 | 540 | 179 | − | Baculoviral IAP repeat-containing | 30.67 | 3.00 × 10⁻¹⁴ | |
| | | | | | | protein 2 [Armadillidium vulgare] | | | |
| | | | | | | (RXG68825.1) | | | |
| 88 | 101084 | 101866 | 783 | 260 | − | Inhibitor of apoptosis protein | 39.23 | 2.00 × 10⁻⁴⁶ | |
| | | | | | | [Penaeus monodon] (AIG94824) | | | |
| 89 | 102157 | 105006 | 2850 | 949 | − | hypothetical protein PmNV-003 | 27.6 | 2.00 × 10⁻⁹¹ | |
| | | | | | | [Penaeus monodon nudivirus] | | | |
| | | | | | | (YP_009051841.1) | | | |
| 90 | 105353 | 105763 | 411 | 136 | + | E3 ubiquitin-protein ligase XIAP | 52.17 | 2.00 × 10⁻⁰⁹ | |
| | | | | | | [Pan panicus] (XP_014198343.2) | | | |
| 91 | 106036 | 106866 | 831 | 276 | + | baculoviral IAP repeat containing | 32.92 | 5.00 × 10⁻²⁹ | |
| | | | | | | protein 2-like [Penaeus monodon] | | | |
| | | | | | | (XP_037774584.1) | | | |
| 92 | 107219 | 108046 | 828 | 275 | + | — | | | |
| 93 | 108350 | 109000 | 651 | 216 | − | — | | | |
| 94 | 109079 | 111745 | 2667 | 888 | − | hypothetical protein PmNV-003 | 27.01 | 1.00 × 10⁻⁷⁸ | |
| | | | | | | [Penaeus monodon nudivirus] | | | |
| | | | | | | (YP_009051841.1) | | | |
| 95 | 111803 | 112291 | 489 | 162 | − | — | | | |
| 96 | 112402 | 113694 | 1293 | 430 | + | Polyhedrin [Penaeus monodon | 49.66 | 1.00 × 10⁻¹³¹ | |
| | | | | | | nudivirus] (ABY75164.1) | | | |
| 97 | 113956 | 114639 | 684 | 277 | − | — | | | |
| 98 | 115484 | 116620 | 1137 | 378 | − | MAM and LDL receptor class A | 61.54 | 2.00 × 10⁻⁰⁶ | |
| | | | | | | domain containing protein 1 | | | |
| | | | | | | [Salmotrutta] (XP_029611939.1) | | | |
| 99 | 116701 | 118818 | 2118 | 705 | + | low density lipoprotein receptor | 43.69 | 9.00 × 10⁻¹³ | |
| | | | | | | related protein [Apis cerana cerana] | | | |
| | | | | | | (PBC27447.1) | | | |
| 100 | 118969 | 119511 | 543 | 180 | + | uncharacterized protein | 44.86 | 2.00 × 10⁻²¹ | |
| | | | | | | LOC119569943 [Penaeus monodon] | | | |
| | | | | | | (XP_037773819.1) | | | |

### Tandem Repeats

A total of 13 tandem repeat regions were identified along the genome of PvSNPV. Three out of thirteen tandem repeats partially overlapped 2 predicted ORFs (FIG. 2) (Table Si). The repeat region length ranged from 60 to 230 bp.

TABLE S1
The tandem repeat distribution along PvSNPV genome. The
tandem repeats were found using Tandem Repeat Finder
(https://tandem.bu.edu/trf/output/127ZAv7Pv8JVZ.2.7.7.80.10.100.500.1.html,
default, score > 100).

| | Period | Copy | Consensus | Percent | Percent | | | | | | Entropy |
|---|---|---|---|---|---|---|---|---|---|---|---|
| Indices | Size | Number | Size | Matches | Indels | Score | A | C | G | T | (0-2) |
| 20395--20527 | 28 | 5.1 | 28 | 72 | 20 | 135 | 27 | 8 | 16 | 48 | 1.74 |
| 20399--20527 | 52 | 2.5 | 51 | 94 | 2 | 224 | 27 | 8 | 16 | 48 | 1.75 |
| 26798--26894 | 45 | 2.2 | 43 | 92 | 5 | 160 | 28 | 25 | 7 | 38 | 1.83 |
| 27104--27186 | 28 | 3 | 28 | 83 | 0 | 121 | 39 | 14 | 21 | 24 | 1.91 |
| 28366--28433 | 31 | 2.2 | 31 | 89 | 0 | 109 | 47 | 8 | 19 | 25 | 1.78 |
| 28788--28853 | 33 | 2 | 33 | 96 | 0 | 123 | 31 | 13 | 27 | 27 | 1.94 |
| 62814--62967 | 31 | 5 | 31 | 90 | 0 | 236 | 28 | 20 | 2 | 48 | 1.62 |
| 62814--62967 | 62 | 2.5 | 62 | 94 | 0 | 263 | 28 | 20 | 2 | 48 | 1.62 |
| 97464--97577 | 28 | 4.1 | 28 | 100 | 0 | 228 | 24 | 14 | 17 | 42 | 1.87 |
| 100630--100689 | 30 | 2 | 30 | 96 | 0 | 111 | 16 | 35 | 10 | 38 | 1.82 |
| 105028--105108 | 29 | 2.8 | 29 | 88 | 0 | 126 | 24 | 2 | 18 | 54 | 1.56 |
| 105173--105257 | 30 | 2.8 | 30 | 76 | 11 | 102 | 20 | 8 | 16 | 55 | 1.66 |
| 107494--107723 | 114 | 2 | 114 | 81 | 5 | 268 | 42 | 9 | 17 | 30 | 1.82 |

### Orthologous and Core Gene Analyses

The putative protein sequences from 15 nudiviruses, 5 baculoviruses and PvSNPV were analyzed using OrthoFinder v2.5.4. The results showed that 1982 (69.6%) out of 2846 protein sequences were assigned into 418 orthogroups. Fifty percent of all genes were categorized in orthogroups, with at least 4 genes per group (G50=4), and these genes were contained in the largest 178 orthogroups (050=178). Interestingly, the results showed that 27 to 80 genes from PvSNPV in the orthogroups clustered with the genes from nudiviruses, meanwhile only 3 to 9 genes from PvSNPV were in the orthogroups with genes originating in baculoviruses. In particular, eighty genes from PvSNPV were in orthogroups with 80 genes from P. monodon nudivirus (PmNV). Table 2 shows species overlap in orthogroups of baculoviruses and nudiviruses. The number represented the number of genes from each species in orthogroups.

The reciprocal BlastP and orthologous analyses showed that 25 out of 28 nudivirus core genes were identified in PvSNPV (Table 3). Five ORFs involved in DNA repair and replication (i.e., dna polymerase, integrase, two helicase-2, and a helicase) were identified in the PvSNPV genome (ORF1 (id %=59.89; E=0.00), ORF39 (id %=61.06; E=1.00×10⁻¹³⁸) ORF59 (id %=53.62; E=0.00), ORF62 (id %=49.86; E=0.00), and ORF76 (id %=52.54; E=0.00)) (Table 1). In addition, orthologous analyses also showed the deduced amino acid sequence from ORF12 was in the orthogroup as FEN-1, which is responsible for DNA repair. Interestingly, helicase-2 is a duplication event gene in PvSNPV genome. Four ORFs, which are predicted to be involved in transcription in nudiviruses, were also identified in the PvSNPV genome. The deduced amino acid sequences from ORF7, ORF15, ORF42, ORF73 matched with P47 (id %=50.83; E=4.00×10⁻¹⁵¹), LEF-8 (id %=57.23; E=0.00), LEF-9 (id %=61.76; E=0.00), LEF-4 (id %=48.69; E=4.00×10⁻¹⁵⁷, respectively (Table 1). Orthologous analyses revealed that deduced amino acid sequence from ORF36 was in LEF-5 annotated orthogroup. The deduced amino acid sequences from ORF5, ORF8, ORF24, ORF56, ORF70, ORF75, and ORF77 were homologous to ODV-E56 (id %=57.01; E=5.00×10⁻¹⁷⁷), PIF-2 (id %=61.6; E=3.00×10⁻¹⁷¹), PIF-1(id %=52.85; E=0.00), P74 (id %=57.56; E=0.00), PIF-6 (id %=47.55; E=9.00×10⁻⁴³), PIF-3 (id %=51.03; E=2.00×10⁻⁶⁵), ODV-E28 (id %=43.12; E=1.00×10⁻⁶³), respectively (Table 1). Those proteins were well documented to be involved in per os infection [27]. The genes involved in viral packaging, assembly and release were also identified in PvSNPV genome. For instance, the deduced amino acids from ORFs 81, 43, 68, 4, 3, and 72 were homologous to the 11K-like (id %=59.79; E=2.00×10⁻³⁵), 38K (id %=50; E=3.00×10⁻⁸⁵), AC-81(id %=63.4; 8.00×10⁻⁶⁷), VP91(id %=48.15; E=0.00), AC92 (id %=53.43; E=1.00×10⁻⁷²) and VLF-1 (id %=56.17; E=1.00×10⁻⁷⁹) proteins, respectively. However, vp39 and p6.9 genes were not identified in PvSNPV genome even though these are present in other nudiviruses [22](Table 3).

TABLE 2
The species overlap in orthogroups of baculoviruses and nudiviruses. The
number represented the number of genes from each species in orthogroups.

| | AcMNPV | BmNPV | CnNPV | CpGV | LxMNPV | CcNV | CmNV | DhNV | DiNV | ENV | GbNV |
|---|---|---|---|---|---|---|---|---|---|---|---|
| AcMNPV | | 133 | 19 | 52 | 85 | 4 | 6 | 3 | 2 | 2 | 3 |
| BmNPV | 133 | | 18 | 52 | 81 | 4 | 6 | 3 | 2 | 2 | 3 |
| CnNPV | 19 | 18 | | 19 | 20 | 3 | 3 | 3 | 3 | 3 | 3 |
| CpGV | 52 | 52 | 19 | | 54 | 3 | 7 | 4 | 5 | 5 | 6 |
| LxMNPV | 85 | 81 | 20 | 54 | | 4 | 6 | 4 | 3 | 3 | 4 |
| CcNV | 4 | 4 | 3 | 3 | 4 | | 57 | 57 | 24 | 23 | 23 |
| CmNV | 6 | 6 | 3 | 7 | 6 | 57 | | 59 | 25 | 25 | 25 |
| DhNV | 3 | 3 | 3 | 4 | 4 | 57 | 59 | | 26 | 25 | 25 |
| DiNV | 2 | 2 | 3 | 5 | 3 | 24 | 25 | 26 | | 77 | 54 |
| ENV | 2 | 2 | 3 | 5 | 3 | 23 | 25 | 25 | 77 | | 50 |
| GbNV | 3 | 3 | 3 | 6 | 4 | 23 | 25 | 25 | 54 | 50 | |
| HgNV | 7 | 7 | 3 | 6 | 7 | 58 | 80 | 60 | 25 | 25 | 25 |
| HzNV-1 | 6 | 6 | 3 | 8 | 8 | 33 | 36 | 33 | 27 | 26 | 26 |
| HzNV-2 | 6 | 6 | 3 | 8 | 8 | 33 | 36 | 33 | 27 | 26 | 26 |
| KNV | 2 | 2 | 3 | 5 | 3 | 23 | 24 | 25 | 85 | 76 | 53 |
| MNV | 2 | 2 | 3 | 5 | 3 | 23 | 25 | 25 | 88 | 78 | 53 |
| OrNV | 5 | 5 | 3 | 9 | 7 | 25 | 28 | 28 | 80 | 76 | 55 |
| PmNV | 5 | 5 | 3 | 5 | 5 | 58 | 75 | 60 | 27 | 25 | 27 |
| PvSNPV | 6 | 6 | 3 | 6 | 6 | 55 | 73 | 56 | 25 | 25 | 25 |
| TNV | 4 | 4 | 3 | 8 | 6 | 24 | 27 | 27 | 78 | 75 | 54 |
| ToNV | 4 | 4 | 3 | 4 | 4 | 28 | 33 | 31 | 25 | 24 | 27 |

| | HgNV | HzNV-1 | HzNV-2 | KNV | MNV | OrNV | PmNV | PvSNPV | TNV | ToNV |
|---|---|---|---|---|---|---|---|---|---|---|
| AcMNPV | 7 | 6 | 2 | 2 | 2 | 5 | 5 | 6 | 4 | 4 |
| BmNPV | 7 | 6 | 2 | 2 | 2 | 5 | 5 | 6 | 4 | 4 |
| CnNPV | 3 | 3 | 3 | 3 | 3 | 3 | 3 | 3 | 3 | 3 |
| CpGV | 6 | 8 | 8 | 5 | 5 | 9 | 5 | 6 | 8 | 4 |
| LxMNPV | 7 | 8 | 8 | 3 | 3 | 7 | 5 | 6 | 6 | 4 |
| CcNV | 58 | 33 | 33 | 23 | 23 | 25 | 58 | 55 | 24 | 28 |
| CmNV | 80 | 36 | 36 | 24 | 25 | 28 | 75 | 73 | 27 | 33 |
| DhNV | 60 | 33 | 33 | 25 | 25 | 28 | 60 | 56 | 27 | 31 |
| DiNV | 25 | 27 | 27 | 85 | 88 | 80 | 27 | 25 | 78 | 25 |
| ENV | 25 | 26 | 26 | 76 | 78 | 76 | 25 | 25 | 75 | 24 |
| GbNV | 25 | 26 | 26 | 53 | 53 | 55 | 27 | 25 | 54 | 27 |
| HgNV | | 36 | 36 | 24 | 25 | 28 | 74 | 72 | 27 | 33 |
| HzNV-1 | 36 | | 102 | 25 | 27 | 28 | 35 | 36 | 28 | 30 |
| HzNV-2 | 36 | 102 | | 25 | 27 | 28 | 35 | 36 | 28 | 30 |
| KNV | 24 | 25 | 25 | | 86 | 80 | 26 | 24 | 78 | 24 |
| MNV | 25 | 27 | 27 | 86 | | 81 | 26 | 25 | 79 | 25 |
| OrNV | 28 | 28 | 28 | 80 | 81 | | 30 | 28 | 89 | 26 |
| PmNV | 74 | 35 | 35 | 26 | 26 | 30 | | 80 | 28 | 33 |
| PvSNPV | 72 | 36 | 36 | 24 | 25 | 28 | 80 | | 27 | 33 |
| TNV | 27 | 28 | 28 | 78 | 79 | 89 | 28 | 27 | | 25 |
| ToNV | 33 | 30 | 30 | 24 | 25 | 26 | 33 | 33 | 25 | |

TABLE 3
A list of identified core genes across PvSNPV and fifteen nudiviruses.
The number indicated the ORF number in each nudivirus genome.

| Gene | Description | PvSNPV | PmNV | CcNV | CmNV | DhNV | DiNV | ENV |
|---|---|---|---|---|---|---|---|---|
| DNA | | | | | | | | |
| replication, | | | | | | | | |
| repair | | | | | | | | |
| dnapol | DNA polymerase | 1 | 5 | 1 | 1 | 1 | 65 | 1 |
| helicase | DNA helicase | 76 | 94 | 93 | 83 | 95 | 12 | 42 |
| helicase-2 | DNA helicase | 59, 62 | 76, 79 | 77, 89 | 66, 69 | 87, 90 | 90 | 65 |
| fen-1 | FEN-1/FLAP | 12 | 20 | 12 | 13 | 12 | 100 | 56 |
| | endonuclease | | | | | | | |
| integrase | DNA processing | 39 | 55 | 56 | 40 | 54 | 39 | 18 |
| Transcription | | | | | | | | |
| lef-4 | RNA polymerase | 73 | 91 | 90 | 80 | 92 | 18 | 36 |
| | subunit | | | | | | | |
| lef-5 | Transcription | 36 | 52 | 53 | 38 | 51 | 25 | — |
| | initiation | | | | | | | |
| | factor | | | | | | | |
| lef-8 | RNA polymerase | 15 | 23 | 15 | 16 | 15 | 32 | 24 |
| | subunit | | | | | | | |
| lef-9 | RNA polymerase | 42 | 58 | 60 | 46 | 56 | 57 | 9 |
| | subunit | | | | | | | |
| p47 | RNA polymerase | 7 | 14 | 8 | 8 | 7 | 104 | 53 |
| | subunit | | | | | | | |
| Oral | | | | | | | | |
| infection | | | | | | | | |
| pif- | Per os infectivity | 56 | 72 | 74 | 63 | 71 | 74 | 80 |
| 0/p74 | factor | | | | | | | |
| pif-1 | Per os infectivity | 24 | 39 | 35 | 27 | 39 | 35 | 21 |
| | factor | | | | | | | |
| pif-2 | Per os infectivity | 8 | 15 | 9 | 9 | 8 | 101 | 55 |
| | factor | | | | | | | |
| pif-3 | Per os infectivity | 75 | 93 | 92 | 82 | 94 | 91 | 64 |
| | factor | | | | | | | |
| pif- | Per os infectivity | 77 | 96 | 94 | 84 | 96 | 11 | 43 |
| 4/odv- | factor | | | | | | | |
| e28 | | | | | | | | |
| pif- | Per os infectivity | 5 | 10 | 6 | 6 | 5 | 6, 85 | 69 |
| 5/odv- | factor | | | | | | | |
| e56 | | | | | | | | |
| pif-6 | Per os infectivity | 70 | 88 | 85 | 77 | 79 | 36 | 20 |
| | factor | | | | | | | |
| Viral | | | | | | | | |
| packaging, | | | | | | | | |
| assembling | | | | | | | | |
| and releasing | | | | | | | | |
| 11K- | Occlusion body | 81 | 100 | 101 | 88 | 101 | 17 | 37 |
| like | component | | | | | | | |
| 38K | Nucleocapsid | 43 | 59 | 61 | 47 | 60 | 46 | 14 |
| | protein | | | | | | | |
| ac81 | Nucleocapsid | 68 | 86 | 83 | 75 | 81 | 62 | 4 |
| | envelopment | | | | | | | |
| vp39 | Major capsid | — | 22 | 14 | 15 | 14 | 99 | 57 |
| | protein | | | | | | | |
| p6.9 | Nucleocapsid | — | — | 106 | 99 | — | 1 | 51 |
| | packaging/ | | | | | | | |
| | assembly | | | | | | | |
| vp91/p95 | Nucleocapsid | 4 | 9 | 5 | 5 | 4 | 92 | 63 |
| | protein | | | | | | | |
| p33_ac92 | Sulfhydryl | 3 | 8 | 4 | 4 | 3 | 87 | 67 |
| | oxidase | | | | | | | |
| vlf-1 | Very late gene | 72 | 56 | 59 | 41 | 55 | 9 | 45 |
| | expression factor | | | | | | | |
| Unknown | | | | | | | | |
| function | | | | | | | | |
| GbNV_{—} | Unknown | 79 | 98 | 98 | 86 | 99 | 22 | 32 |
| gp19-like/ | | | | | | | | |
| esterase | | | | | | | | |
| GbNV_{—} | Unknown | — | 62 | 64 | 50 | 63 | 34 | 22 |
| gp51-like | | | | | | | | |
| GbNV_{—} | Unknown | 80 | 99 | 99 | 87 | 100 | 102 | 54 |
| gp67-like | | | | | | | | |

| | | | HzNV- | HzNV- | | | | | |
|---|---|---|---|---|---|---|---|---|---|
| Gene | GbNV | HgNV | 1 | 2 | KNV | MNV | OrNV | TNV | ToNV |
| DNA | | | | | | | | | |
| replication, | | | | | | | | | |
| repair | | | | | | | | | |
| dnapol | 12 | 1 | 131 | 18 | 10 | 1 | 1 | 1 | 12 |
| helicase | 88 | 84 | 104 | 38 | 49 | 45 | 34 | 26 | 118, 55 |
| helicase-2 | 46 | 66, 69 | 60 | 76 | 78 | 71 | 108 | 74 | 105 |
| fen-1 | 65 | 13 | 68 | 70 | 68 | 62 | 16 | 17 | 1 |
| integrase | 57 | 42 | 144 | 8 | 25 | 21 | 75 | 54 | 43 |
| Transcription | | | | | | | | | |
| lef-4 | 96 | 81 | 98 | 43 | 43 | 39 | 42 | 33 | 25 |
| lef-5 | 85 | 39 | 101 | 40 | — | — | 52 | 40 | 50, 66 |
| lef-8 | 49 | 16 | 90 | 51 | 33 | 29 | 64 | 46 | 88 |
| lef-9 | 24 | 46 | 75 | 63 | 4 | 8 | 96 | 65 | 131 |
| p47 | 69 | 8 | 75 | 63 | 64 | 58 | 20 | 13 | 115 |
| Oral | | | | | | | | | |
| infection | | | | | | | | | |
| pif- | 45 | 63 | 11 | 106 | 95 | 88 | 126 | 88 | 45 |
| 0/p74 | | | | | | | | | |
| pif-1 | 52 | 28 | 55 | 82 | 29 | 25 | 60 | 49 | 69 |
| pif-2 | 66 | 9 | 123 | 26 | 67 | 61 | 17 | 16 | 7 |
| pif-3 | 3 | 83 | 88 | 53 | 77 | 70 | 107 | 73 | 13 |
| pif- | 87 | 85 | 103 | 39 | 51 | 46 | 33 | 25 | 119 |
| 4/odv- | | | | | | | | | |
| e28 | | | | | | | | | |
| pif- | 5 | 6 | 76 | 62 | 56, 84 | 51, 76 | 115 | 77 | 102, |
| 5/odv- | | | | | | | | | 74, 96 |
| e56 | | | | | | | | | |
| pif-6 | 55 | 78 | 74 | 64 | 28 | 24 | 72 | 51 | 56 |
| Viral | | | | | | | | | |
| packaging, | | | | | | | | | |
| assembling | | | | | | | | | |
| and releasing | | | | | | | | | |
| 11K- | 95 | 89 | 124 | 25 | 44 | 40 | 41 | 32 | 28 |
| like | | | | | | | | | |
| 38K | 1 | 47 | 10 | 108 | 20 | 14 | 87 | 59 | 63 |
| ac81 | 14 | 76 | 33 | 96 | 7 | 4 | 4 | 4 | 12 |
| vp39 | 64 | 15 | 89 | 52 | 69 | 63 | 15 | 18 | 87 |
| p6.9 | 72 | 40 | 142 | — | 61 | 56 | 22 | 11 | 51 |
| vp91/p95 | 2 | 5 | 46 | 89 | 76 | 69 | 106 | 72 | 16, 83 |
| p33_ac92 | 7 | 4 | 13 | 104 | 82 | 74 | 113 | 75 | 99 |
| vlf-1 | 80 | 43 | 121 | 28 | 53 | 48 | 30 | 24 | 65 |
| Unknown | | | | | | | | | |
| function | | | | | | | | | |
| GbNV_{—} | 19 | 87 | 30 | 99 | 39 | 35 | 47 | 38 | 27 |
| gp19-like/ | | | | | | | | | |
| esterase | | | | | | | | | |
| GbNV_{—} | 51 | 50 | — | 79 | 30 | 26 | 61 | 48 | 19 |
| gp51-like | | | | | | | | | |
| GbNV_{—} | 67 | 88 | 122 | 27 | 66 | 60 | 18 | 15 | 6 |
| gp67-like | | | | | | | | | |

The ORF information was obtained from Biezier et al., 2015 and Bateman et al., 2021

Two out of three unknown functional core genes present in nudiviruses were also identified in the PVSNPV genome. The deduced amino acid sequence of ORF79 was homologous to the ESTERASE protein, with identity percentage of 43.12 and E-value of 10⁻⁶³. Additionally, orthologous analyses revealed that deduced amino acid sequence from ORF80 was in the orthogroup with GBNV-GB67-like protein, which is one of the nudivirus unknown functional core proteins. However, Applicant was not able to identify a GbNV-gb51-like gene in PvSNPV genome.

### Phylogenetic Analyses

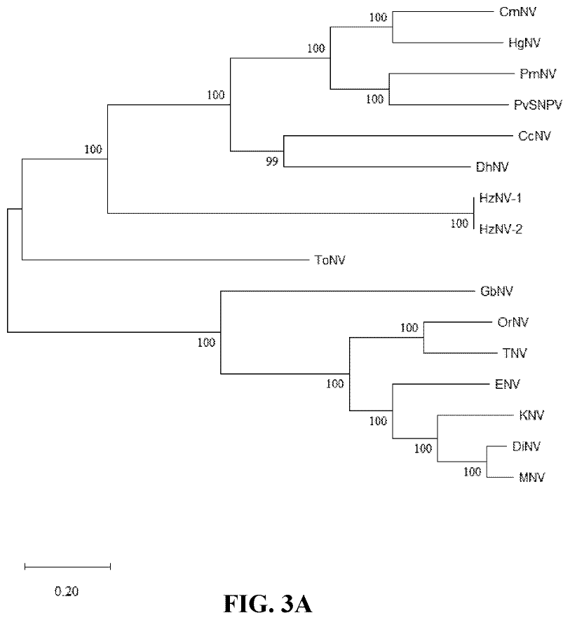

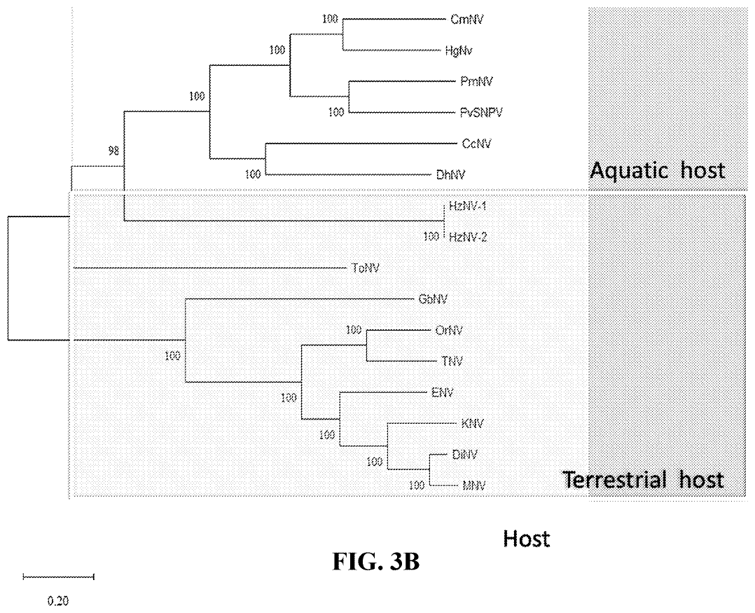

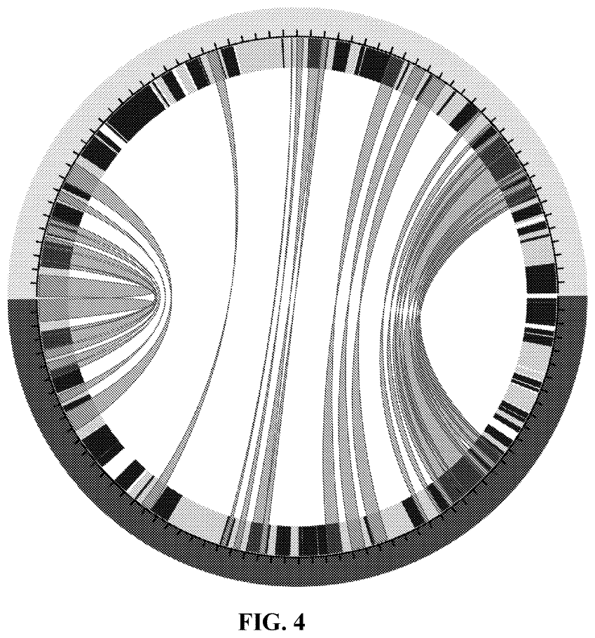

The phylogenetic tree based on concatenated orthologous protein sequences (i.e., PIF-2, PIF-0/P74, DNAPOL, AC-92, FEN-1, LEF-8, PIF-1, INTEGRASE, LEF-9, 38K, AC-81 LIKE, PIF-6, LEF-4, PIF-3, ODV-E28, and GB-67-LIKE) clustered PvSNPV with the same clade as PmNV with a bootstrap value of 100% (FIG. 3A). Likewise, the core proteins based phylogenetic tree also showed that PvSNPV belongs to the same clade with PmNV with bootstrap values of 100% (FIG. 3B). Interestingly, PvSNPV and other nudiviruses infecting crustaceans were in a separate clade from nudiviruses infecting terrestrial animals (FIG. 3B). In addition, PmNV and PvSNPV core genes showed similar orientation (FIG. 4).

### Polyhedrin Promoter Analysis

The nucleotide sequence upstream of the polyhedrin gene from eight nudiviruses, including CmNV, CcNV, KNV, MNV, OrNV, PmNV, ToNV and PvSNPV, were analyzed by the NNPP server to search for promoters.

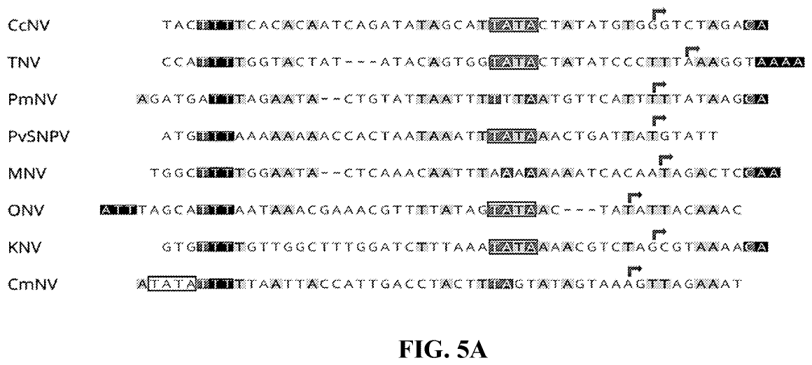

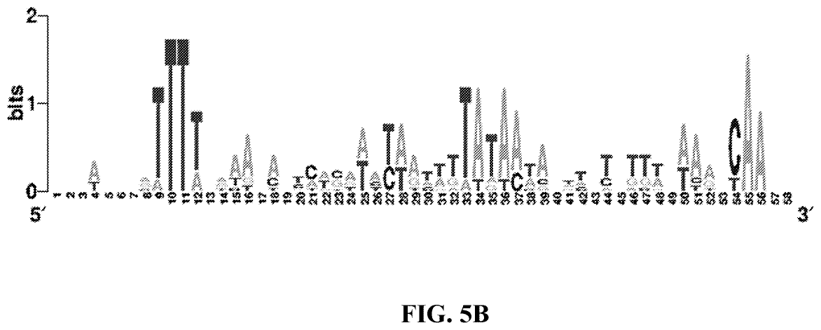

Putative promoter regions were identified in all eight viruses and the TATA boxes were identified in 6 out of 8 putative promoter regions. (FIG. 5A). The TATA boxes were located at −10 to −36 positions. Multiple alignment of polyhedrin promoters from 8 nudiviruses revealed that the polyhedrin promoters contained the consensus sequences in ~20 bp spacing context (i.e., TTTT . . . TATAA) (FIG. 5B)

Below is an analysis of different ORFs and related promoter regions. For the following, the different regions are identified as follows:

Italicized: Sequence upstream of promoter element

Bolded and underlined: Putative promoter element

Underlined: Predicted open reading frame

Double underlined, bolded, and italicized: Transcription start site

Double underlined and bolded: TATA box

>11k Protein
AAATTAACATTGAAAGTTAAAATGTCTTTTCAATTTCCGTAAATCTTGAA
TAATAATTTCTA [symbol] CGGATTGAAATCAATTAAGTTAATAGCGACTTACAAGATGC
AAACGGCAAGATCCTATATGAAACTTGAATGTACTGATATTTCCAAGTCATTACA
GATCTCTGCAATCTTGCAGATGATGATTACCGCAATTATATTTATTATGTTGATCT
TTGCAGCTAGTTATAACAATATGGTTAACCCTACAGGGGATGAAGGTATGGAAA
AGAAGGAAAAGTATATTAACGGTCTGGTGATATCTTCGGCCGTTATTAGTACACT
TTTATTCATTATTGGTGTTTGGCATGTATATGTTGCGATCAAAGCCAAAAAATGC
ATTACGACAACTCCAGTAACTCGTACTTAA (SEQ ID NO: 1)
>38k protein
AGTGGAATCTGCAAGCTTTTGTTTCTTTGCAATAGACTCTATTGCTGAAGTTT
TGAGTTTACGCTTTCCAGTGGTGGTGGTGATTTTCGGAGAAGGCTTTTTACTAT
AGGAAAAGCTGGGCTTATCGACTTCACTCACGTCATCAGTATCAGAAGAGTCTT
CACAGTATAAATCTGGGTCCATATCTATAAATAAAAAAAATGAAACAAATTGTA
TTTATAAATTTTGTAAATGTACAAGATTTGAATATGGATGATATTTACAACTTACT
AATGACGTGTTTCCAACATTTTACGCACATGTACATTCTACATGGCTCATTTGCTA
GATGCTTTTTCCAAGATGCTAAACGTAAAAGGGTCAATCATGAAGATGATTTACC
TGGTATTTACTTTATAAAGAATATTCACAATAACATAAAATGCTACAGTATGCTC
ATAAATGTAAACGATTTCAACTTTGTATATAGTAATAAATATACACATGTTGATT
ACAAAAATCTGAGCGTTGGTAAGAATTTTGATTTCAATGCACTCTCGAAAGAACT
ATCTTTGAACCTGAGATCCTTTTTTAGAACTAACGCTAATAAAAAGAAATCTATA
GCAGTGATCGATTTGGATGATACAATCATAAACAAAAATGGTGATTTTATATTGG
AACCCTTTGACAAATATTTAGCCACACTTAAAAATACATTTGATTTAGTTGTGTTG
TGGTCTCATGGATGCCAACAACATGTCAACCATGTATTCTCAAACACTTTGAATG
GTTATGCAAAAGAATTTGATATGATTATGGTTCGTAGCAGTTCAGTCCAAATAAG
TAACAAGGGTAAGGGATTCTTACTTCAACTGATGAACCAAGCTTTTGGAGTTATT
GAATTTACATATTCTGTATTGATAGATGACCAGGCGTACAATTATAAGAATGATT
ATGACTGTTTTATACATGTACCAAAATGTTTATCACATAAAGCATACAATAAGCA
AATGTGGAACATGTTGTATAAACTGAAAAATACTATAGATAAAAGACTAAACTT
GTAA (SEQ ID NO: 2)
>ac81 Protein
CTGCCATTTCAATAAAGTTTAAAGTATCATAACATATAATAATTGCAAAATTTG
AACCCCATAAAGAGTCACCAATATTAAAATAATTCAAATCTTCAGTATATGGTGTT
ATCATTTTACTATTAAAATCATAATAGGTTTTGATATCTAGATTTCTTAGCTTTG
TAAAAGTGCAATTTCCATCTCTATCACCAAAGTAGATGGAAAACGAATCCGACA
TTGTAAGTCTTAATACTAAAAATTATGATGTACTTATTTATTCAAAATCAATAAA
GAATACACTAAATTTATTTATTCATAATTATCTTTCAATTCCTGCACTTGAACTAG
AAATCCACCCCGGTAGATTTTATTATGGTACCCACCATACAATGGGGGCTTTTAT
AAAGAGAAGTGTCATAATTAAGAAGCTGCAATTTTGTGAAGACTGTATAAACAA
GTTATTAAAACAGTCTTACGACCTTTTTAAGGTTTGGTATTACCCGATAGTGAATT
GCGAGACTCTAACTCGTGGCTTAACACAAAACATACCAATAAGTATACAAACAA
TTTTAATAACTGGAGCATTTACAAGTCTTGTTATTGGTATATCATACCCTTCTGTA
TTTCTATTAACATTTTTATTTATAATTTTGTTATTGTTATACAATAACTTTCGTTTC
CAACTTTTTAGAAGCACTTGTATTCATAACCCACAAGTATGA (SEQ ID NO: 3)
>polH Polyhedrin Protein
GATAATTAAATCTTTATGTTTAAAAAAAACCACTAATAAATTTATAAAC
TGATTATGTATTTATGTATTTCATTATGTTGAAAATAAAATCTTTTAAAATGTTA
TTTTTTTTAATCTTTCTAACATTTTTATAAAATTTGTAATTTGTGCGAGTAAACCTA
TGTAGCAGCAGAGAAGAGAGAAATAAGTGAAGTAAATAAAATGTCGGGAGCTG
AACATATGGAAGGTGTTGAGCGGGTGCTCAGGGATTTGAATAATGAGGAAAAAT
TAACACTCGGACTGAGTGCTGTGGGTGGGGTAGTTGGGGCAACTAAGATGATGA
ATGAAGCTGCAGAGCTTAGAAGGAATTATGGAGATCGCCCATATGAAAGGCTTT
TGGAGTTGAGCAAGAGAAAGTATGAGTCCACACCAACATCAGGATTTGGTGAGC
GACTTAGACCATCTATGGCCGAACAGCTAGATAGGAGCACCAAGAGGCCAAGGA
AAGCTAACAGATTCCCAGTAAACACTCATTATAACATGCACAAGATCTGCAAGA
GGACAAACCTGACATCCTCCAAGTTATTAGGCTTTTCTGGCCAGGCTGGACAAGA
CATCCCAAAATATAACAGTGCAGTAACCCTACCATTGGAGGCTCTAGAATTTTGG
GTGGGAGATAACATAAATCCAGAAGTAGCACACTCCATGGGAAGCAAAGTATTG
AGCGATAAGGAATGCAGGGTAAAATCAATGAAACTGAAACTTAGCAACCTGCAA
GTATATGAAGACAGACAATATGGATCTGGAGATAGGCTTAGTGAAACTGCAAAG
GATGTTTCTTGCTTTGTTATTCCAGTAGAATCCTATATGGGTAGCAAATCAAATG
GAACTCTTTGCAAGATGTTCTCGGCCAATACATCAGCAAACAGCAATGTCGGAG
ATTTTATTACAACAAAGGATATCATGATGGGAAAATTAGTTACAAAATCAGATGT
TGATAGAATGAATGTTAACCCAGAAATGTACATGTGGTCATCAGAGGATGGATC
CCTAGAGCTAGAATTCAACCAGGATAACGGGTTGTGGTTTAGGATGCCGGAGCT
TAGAGATGGGGTATACCACTTACTGCCAATGGAATCCGGCATTGGGGCTAGTGC
ATTAGAAACTTACACTATACCAGAAAGTGATGACGAGATCCTAAGAAGTACCAC
CACAATTACACCACTATCATCGGCCATTGGCAGTCTGCTAATTGGAGTACCATAT
ATCATTGATGCCTCTGGAAAGCAAAAGGACTACAGGGTCTCATTTATGGTTGAAC
AGGAAATTACTTTGGAATGCCGCGCTGAATGGATGCAGAGCTCTTCAGCTTCAGA
CTGGAATGCAGGGATGGGTGCTACTCTCACACCAAGATCTACAAATATTACAGA
ATTTAGGCATATGGTTGGACCATACAATGTACAAGCAGAGCATGGAAATAGGAT
GAAAATGCATCAAACTCATCAAGCAGTATAA (SEQ ID NO: 4)
>vp91 protein
GCTGCGAACTCATCAACTGACTTTAGGGGTTTTGAACCAAATTTAATATCGG
CTAACCACTTCATTTCTTTTTAAATAATTGAGGTTTCACCACTAAGAAATATATAATTA
AAAGAAATATGAATAGTATTATAATCAGAGATAACATCTCATTAAAAAAATCT
TTGTATCTCTACAAGACTTAATATACTAAAGAATTCATGAACGTCTTTATCTACTT
GTTGGTTGTTTTTATTTTCATTACAATTTTAATAATTTATTATTTTATACAAAAACC
TAAAAAGTCTAAAAATGTTGAAGATGTTTACTCAAAGTATTTGCTGTCAGAAGAT
ACGCCAGATACAGCATTTACATACAAACGAAGAAGCGATGGTAAATATGAAAAT
TATTATTTGAATTTTGATACGGAAGGGGATCTACTAAAAATGGTAGTTTCTGATT
ACCCAGTTACAAAAACTGGTGAAGATTATAAGCCAAAAGAACATGGAACTTTAA
CTGAAAATGAAAATGGTTTTAGAATAAGTGGAGTTGCTGCAGATTTCAAATGTCC
CGATGGTTGGATTTGGAATAGTAATAAAAAGACATGTAGTCTGATACCCATTTGT
GGAAGTGATGACGAGGGTAAGATAAAAGGGTTGGATTTTTACTACTTTAATCTAG
CAAAATCCATGGGTCTCATAAAGTCTGCTACAAAATACCATGAACGGTTATATAT
TACTTGCCTGGAAGACCAGTCTTATAATATAAAAACTTGTCCAGACAATATGCTC
TTTAACGAGTTACCAATCCAAGATGATACTGGCGAACTACCTTGCAAGTTTTATG
ATATCTGTGAAGGTATGAGAAACTACGAATTTCATAGACAACAGATCTCGGAAG
ATGTTGTTTTGGAGCCTAGTCAATATTATATGTGCTTTAACGGTATTAGTAATCTG
AAGCAATGTGAAGAGGGTGCATTTTTCAATGAAGAATTGGGTGGTTGCTTAGAA
GAAAATCAATGTATGGGCAAACCAGACGGTTTCACACTACCATCAGATTTAAATT
CGTACATCCTATGTCAAAATGAACAAGAGTATAGCATATATTGCAAAGATGGTAT
ATATGAAGCGCTCGGCCCCAATGCACTCAGTTGTAGTATTGATAAATCCCAGACA
TATTTCAAGTATTTTACAAATGATTTTATTAGCATACCCATTGGGCTATATGTATA
CAAAAATAATCAAAAAAGTGAGAGGTATGCACAAACCGAAATGGTCAAAAAAT
CTATGGCTCTAGAACCATCAACTTCAAGATTTTTCGGTGCGGTTAGAAATGATAT
GCTGTATGACCCAGTAGAATACCAAAAGTATTTTATAACATACTTAGATAATACA
ACCCTAGAAGATTCGGTGGAAGTTGAACTGGACAAGACAAACTATCAAATATTT
AACCCAAAAGCTCTAGTATCTGCATGTTATTATGTTCACAGGTTACCAGCTTTCG
ATTGGAATATATTTGAAGATGTTCCTGTGTCTGATTATGTGAATAAAGAGTCATC
GTATTACTACAAGTATGACAAAGTTATAAAACATACAAAAGATGATACATTTTCT
GAAAATAGCATAGATTACTTCTTCTTTTGTACAGCACAGCATTTGTATAAACCAG
TTGATCCAACAGTTATAGTTACCGATGAAGCGTCTGGTATATTATGTTATGCGGA
TTTTAAAATGTCTAGATTTGGCATATCTTCAAATTATAGTAAAGCATTCACCGTAT
TAAAATTTGCAAATATGGGAGATTCTACAATTTTATACTATGTTGATCCATACGA
AAATGCTATGATTGCTGCAGCTTTTGACAATTCCGTTATATTATCGGATTTTGTAA
ATGTTACAGATGATGGGTATACATGTACGCCCAAGATGTTTACATATGATGAGTT
GAAAAATGATAAGCCAAATGAGTATTTTTACATAAGAATGTCATCGATAAAATA
TGATGGAGTGGTTGATAACACTATGAGTAGATATGTGTTGCCTGAAATATTATTA
CTTGTCGACTTAAAAGTTGCTACGCAAATGGGTAAATTCTTTACAATTTTAAATA
CTGAAAACCTAAATATTGCATCTGATTTAACTGTTTTTAGTACTATAGTAGAAGA
TTCATTTATACCAATAACTGAACATACTGAAAATATATCATATTTTGAAAACGTT
TTAGATTTGCTTTACCAGGATCATGTTTCAAACGGTAGTTCGTGA (SEQ ID NO: 23)
Promoter Sequences (from above):
11k Protein Promoter (SEQ ID NO: 5):
TGTCTTTTCAATTTCCGTAAATCTTGAATAATAATTTCTA [symbol] CGGAT
38k protein promoter (SEQ ID NO: 6):
GGTGGTGGTGATTTTCGGAGAAGGCTTTTTACTATAGGAAAAGCTG
polH Polyhedrin Protein promoter (SEQ ID NO: 7):
ATGTTTAAAAAAAACCACTAATAAATTTATAAACTGATTATGTATT
vp91 protein promoter (SEQ ID NO: 8):
TATATAATTAAAAGAAATATGAATAGTATTATAATCAGAGATAACA

### Discussion

Penaeus vannamei singly enveloped nuclear polyhedrosis virus (PvSNPV, also known as BP) and Penaeus monodon nudivirus (PmNV, also known as MBV) are the first two shrimp viruses identified in 1974 (PvSNPV, Couch, 1974) and in 1977 [28], respectively. Based on transmission electron microscope (TEM) images, both MBV and BP were initially identified as viruses belonging to the family Baculoviridae due to morphological similarities to other baculoviruses. In addition, the presence of occlusion derived viruses in BP and MBV were similar to other members of Baculoviridae family that generate occlusion derived viruses to infect insect species [29]. However, the molecular phylogenetic analysis of six viral genes revealed that the virus called MBV belongs to Nudiviridae family and not to Baculoviridae family, and it is now renamed as a Penaeus monodon nudivirus (PmNV) [27, 30]. Applicant was interested to determine the nucleotide sequence and examine the taxonomic affiliation of PvSNPV at the genome level.

In the present study, PvSNPV was detected in the P. vannamei broodstock held in a quarantine facility during a routine screening. Subsequently, infected broodstock were sacrificed and per os challenge performed using SPF P. vannamei post-larvae to determine the infectivity of the virus detected in the broodstock. When fecal samples of the post-larvae were examined by routine light microscopy, pyramid-like polyhedral occlusion bodies, that are considered characteristic of PvSNPV infection, were observed. In addition, the PvSNPV was also detected in the hepatopancreas of the per os challenged post-larvae by PCR, confirming infectivity of the virus originating in broodstock in the quarantine facility. However, the infectivity of PvSNPV in this study was not as high (20% as determine the PCR positive test), as previously reported (e.g., PvSNPV prevalence is 80% after 6 day infection, [16]. The lower infectivity of PvSNPV in this study was likely due to the developmental stage of the animals used in in the present study (i.e., ~1.0 gm size juvenile) compared to post-larvae used in Hammer's study (i.e., post-larvae 9). In addition, the genetic background of the post-larvae used in the present study is very likely different from the one used by Hammer and colleagues, since many genetic lines of commercially available SPF P. vannamei shrimp tend to show tolerance/resistance to BP. Despite the relatively lower infectivity, the per os experiment confirmed that Applicant was dealing with infectious PvSNPV. The total genomic DNA obtained from the experimentally challenged P. vannamei post-larvae were taken for NGS analysis.

In the 80's through 90's, H&E histology, TEM, biophysical properties of viruses were used to classify shrimp viruses. From 2000 onward, as shrimp viral genomes were sequenced from purified viruses, viral classification were made combining histopathology, biophysical properties of virions and genome sequence data. In recent years, NGS has been utilized to accelerate viral genome sequencing and the availability of large set of sequence data in a relatively short time has further accelerated the use of such data in shrimp virus classification. As a result, shrimp viruses that were previously classified based solely on morphological and histopathological data have been reclassified based on their genomic characteristics. It is for this reason that MBV that was originally classified as a baculovirus is now tentatively assigned as a member of the family Nudiviridae [27]. Similarly, the genome sequence data revealed PvSNPV should be reclassified as a member of the family Nudiviridae, as described in this study.

Applicant used NGS data to characterize PvSNPV that was isolated from P. vannamei broodstock originating in a Latin American country. The circular genome sequence of PvSNPV was approximately 120 kbp, which falls within the range of the genome size of baculoviruses (80-180 kbp) and nudiviruses (97 kbp-230 kbp) [30, 31]. However, orthologous analysis revealed that 27 to 80 genes from PvSNPV were in an orthogroup containing nudiviruses, and only 3 to 9 genes from PvSNPV were in an orthogroup containing baculoviruses, suggesting PvSNPV should be classified as a tentative member of the family Nudiviridae. The members in Nudiviridae family infect several insect and crustacean species and the virus replicates in the nuclei of host cells [32]. The virion of nudivirus is made of cylindrical nucleocapsids which are enveloped to produce a rod-shaped virion displaying a variety of lengths and widths [32]. Initially, the nudiviruses were classified as “non-occluded baculoviruses” (NOBs), due to the similarity in structure sharing between nudiviruses and baculoviruses [33]. Later, nudiviruses were classified as “intranuclear bacilliform viruses (IBVs) after they were removed from Baculoviridae family [33, 34]. Finally, nudiviruses were classified as members of a separate family, Nudiviridae. The name Nudiviridae was coined to describe the lack of occlusion bodies in nudivirus (“nudi” means bare) [22]. However, occlusion bodies have been identified in several nudiviruses such as PmNV, CmNV, CcNV, TNV, KNV, OrNV, and MNV [22, 27, 35, 36], suggesting that occlusion bodies are not a unique characteristic of baculoviruses alone.

Genomic studies revealed thirty one core genes, including homologs from baculoviral core genes that are shared among nudiviruses [37, 38]. A more recent study found that 28 instead of 31 core genes, are known among nudiviruses [22]. In the PvSNPV genome, Applicant could detect twenty five out of these twenty-eight core genes, as proposed by Bateman et al. (2021). We were not able to identify vp39, p6.9 and gb51-like genes in PvSNPV using the BlastP search engine. VP39 is a major capsid protein, which has been annotated in the genomes of MNV [35], HgNV [39], OrNV [36], ToNV [23], ENV, DiNV, TNV, CcNV, and CmNV [22]. Bateman et al., (2021) also found the vp39 gene in PmNV, which was originally identified as ORF22 [27]. Interestingly, in our study, the deduced amino acid sequence of ORF14 in PvSNPV was homologous with the protein sequence from ORF22. Thus, Applicant speculated ORF14 represents the vp39 gene in the PvSNPV genome.

The p6.9 gene, which is responsible for nucleocapsid packaging/assembly, was also not identified in PmNV (Yang et al., 2014). However, upon further analysis of the PmNV genome sequence, Bezier and colleagues detected a p6.9 genes (64,881-65,078) [23]. Likewise, p6.9 has never been annotated in several nudivirus genomes until 2015. The p6.9 gene was recently identified in HzNV-2 (position 24,375-24,127) [23], CcNV (position 72,007-72,231, and CmNV (position 45,460-45,651) [22] using a manual BLAST search. The reason for this could be that the prediction tools are not able to recognize the repetitive serine (S) and arginine (R) as part of potential protein sequence [22]. However, Applicant also employed manual blast search, but Applicant was unable to identify p6.9 gene in PvSNPV genome, suggesting p6.9 gene in PvSNPV is not present unlike many other nudiviruses. The unknown function gene GbNV-gb51-like gene was not identified in both PmNV and PvSNPV genome. However, Bateman et al., (2021) found that ORF62 in PmNV genome was gb51-like gene. Interestingly, ORF46 from PvSNPV was a homolog of ORF62, suggesting the ORF 46 probably represents gb51-like gene.

The Nudiviridae family consists of four genera, which are Alphanudivirus, Betanudivirus, Deltanudivirus and Grammanudivirus. The genera Alphanudivirus, Betanudivirus and Deltanudivirus contain nudiviruses that infect insects, whereas the Grammanudivirus genus has members that infect aquatic hosts. So far, Grammanudivirus genus contains only PmNV and HgNV. Another nudivirus (DhNV) infecting paricarid host has been assigned to Nudiviridae, but the low similarity between DhNV, PmNV and HgNV led the authors to propose a fifth genus, Epsilonnudivirus, for DhNV [40].

Although ICTV just approved PmNV and HgNV as members of the Grammanudivirus genus [32], Bateman's study (2021) showed that the nudiviruses isolated from crustacean species are in a separate clade. The phylogenetic trees constructed from orthologous genes and core genes revealed that PvSNPV falls in the same clade with PmNV (FIG. 3A-3B) with a bootstrap value of 100, and the orientation of core genes was similar in PvSNPV and PmNV (FIG. 4), Therefore and without being bound by theory, Applicant hypothesized PvSNPV, like other nudiviruses isolated from crustaceans, should be classified as a member of Grammanudivirus genus.

Polyhedrin protein is the major component to form occlusion bodies in all baculoviruses and some nudiviruses. The promoter of polyhedrin from AcMNPV has been widely used in recombinant protein expression using baculovirus vectors [41]. Although the polyhedrin gene was identified in several nudiviruses, such as PvSNPV, PmNV, TNV, CmNV, CcNV, KNV, OrNV, and MNV, the promoters of polyhedrin in those nudiviruses have not been identified. In this study, two hundred nucleotides upstream of their start codon were used to identify the polyhedrin promoter using the NNPP server. The results showed that the promoters from tested nudiviruses contained consensus sequences in ~20 bp spacing context. Interestingly, among the promoter elements of eight nudiviruses examined, the polyhedrin promoter from PvSNPV contained a single TATA box, which is similar to the promoter of early expression gene in baculovirus and ToNV [23, 42], suggesting the polyhedrin gene might be an early expressed gene in PvSNPV.

In summary, Applicant sequenced and annotated the full-length genome sequence of PvSNPV, the first viral pathogen reported in shrimp almost 45 years ago. The PvSNPV genome contains a circular double-stranded DNA containing 119,883 bp which encodes 100 putative ORFs. Homologous analysis revealed that PvSNPV belongs to homolog groups with nudiviruses. The results from homologous analysis were supported by core gene sequence data which showed that the PvSNPV genome contained 25 out of the 28 nudivirus core genes. Phylogenetic analysis revealed that PvSNPV falls in the clade of Grammanudivirus. Therefore, Applicant proposes to reassign PvSNPV as a new member of the family Nudiviridae.

### Methods

### PvSNPV Source

Twelve broodstock families were sent to the Aquaculture Pathology Laboratory (APL) at the University of Arizona for quarantine and disease screening from a country in Latin America. Upon arrival, samples were collected for screening OIE-listed crustacean pathogens as well as PvSNPV, Enterocytozoon hepatopenaei (EHP), and Penaeus monodon nudivirus (PmNV) following methods recommended by the OIE and other published papers [6, 17-19]. Some samples turned out to be positive with PvSNPV.

### PvSNPV Challenge Test

A total of 30 shrimp (average weight 1.0 g) were stocked into three 90 L-tanks (10 shrimp/tank) with aeration and biofilter. The hepatopancreas from broodstock that were tested PvSNPV positive were dissected, minced into small pieces and fed to animals in two tanks (1.0 g per tank). The animals in the remaining tank served as a negative control. On day 7 post-infection, five shrimp and feces were independently collected from each tank for PvSNPV screening by wet mount examination and PCR analysis. PCR protocol for PvSNPV detection was obtained from the OIE manual [6].

### Wet Mount Examinations

Sixty microliters of Modified Mayer's Hematoxylin (Thermo Scientific, USA) were put into freshly collected fecal samples placed on a glass slide. The fecal samples were gently spread prior to be covering using a cover slip and observed under a light microscope (Leica DM500, Germany).

### Genome Sequencing

The DNA from PvSNPV positive samples were extracted using DNeasy Blood and Tissue kit (Qiagen, Germany) following manufacture's instruction. A pool (N=5) of extracted DNA was sent for Next generation sequencing (NGS) using an Illumina HiSeq 2500 System (PE 2×150 bp) (Illumina) at OmegaBioservices, Norcross, GA. A library of DNA samples was generated at OmegaBioservices using the Library Kit, KAPA Hyper prep for WGS (Roche).

### Genome Assembly

The DNA reads were trimmed using Trimmomatic v.0.36 (illuminaclip: TruSEQ3-PE, leading:3, trailing:3, minlen:36) [20] and the duplicated reads were removed by Geneious Prime (version 2019, Biomatters, New Zealand). Then, the paired reads were mapped to the P. vannamei genome to remove shrimp genome regions using Geneious Prime (version 2019, Biomatters Ltd, New Zealand), (Low Sensitivities/Fastest). The unused reads were assembled de novo using the Geneious assembler (Medium-Low Sensitivity/Fast) (Geneious Prime v 2019, Biomatters Ltd, New Zealand).

### ORF Prediction and Annotation

The ORFs were predicted using Prokka [21](default, kingdom=virus) via the galaxy server (https://usegalaxy.org/), GeneMarks (Virus, genetic code:11), and fgeneV0 (http://www.softberry.com/, standard code, circular sequence). The results were accepted if they were in agreement with at least 2 of these programs. The deduced amino acid sequences from accepted ORFs were further analyzed by BLASTP, and only ORFs that showed an E-value less than 0.001 were selected. The tandem repeats were found using Tandem Repeat Finder (https://tandem.bu.edu/trf/output/127ZAv7Pv8JVZ.2.7.7.80.10.100.500.1.html, default, score >100). The genome map of PvSNPV was built using Geneious Prime (version 2019, Biomatters, New Zealand).

### Orthologous and Core Gene Analyses

The putative protein sequences of fifteen nudiviruses, five baculoviruses and PvSNPV were subjected to OrthoFinder v2.5.4 (parameter: -A mafft, -M dendroblast, -T fasttree) for identification of orthologous group. Duplication event genes were removed out of orthologous group. The orthologous proteins from each species were concatenated in the same order prior to further analyses. Information of core genes from nudiviruses, which is identified in previous publications [22, 23], and orthologous results were used to identify the core genes in PvSNPV.

### Phylogenetic Analyses

The concatenated orthologous protein sequences were aligned using MAFFT (select an appropriate strategy according to data size) [24, 25] through Geneious Prime (version 2019, Biomatters, New Zealand). A maximum Likelihood phylogenetic tree (JTT matrix-based model, bootstrap=1000) of aligned orthologous protein sequences was built via MEGA-X [26]. Likewise, a maximum Likelihood phylogenetic tree (JTT matrix-based model, bootstrap=1000) of core proteins was also conducted using MEGA-X [26].

### Polyhedrin Promoter Analyses

Two hundred nucleotides upstream of the transcription initiation site of polyhedrin genes of seven nudiviruses (i.e., CmNV, CcNV, KNV, MNV, OrNV, PmNV, and TNV) and PvSNPV were subjected to Neural Network Promoter Prediction (NNPP) server (https://www.fruitfly.org/seq_tools/promoter.html) (type of organism: prokaryote; include reverse strand: No; Minimum promoter score: 0.8). The predicted promoter sequences were subjected to ClustalW2 multiple alignment via Geneious Prime (v2019, Biomatter Ltd, New Zealand). The pattern of Polyhedrin promoter was generated by WebLoGo (http://weblogo.berkeley.edu/).

### References for Example 1

1. Fletcher R. Global shrimp production sees significant growth in 20211 The Fish Site. The Fish site. https://thefishsite.com/articles/global-shrimp-production-sees-significant-growth-in-2021-gorjan-nikolik-rabobank (2021, accessed 17 Dec. 2021).
2. Lightner D V., Redman R M, Pantoja C R, Tang K F J, Noble B L, et al. Historic emergence, impact and current status of shrimp pathogens in the Americas. J Invertebr Pathol 2012; 110:174-183.
3. Asche F, Anderson J L, Botta R, Kumar G, Abrahamsen E B, et al. The economics of shrimp disease. J Invertebr Pathol; 186. Epub ahead of print 1 Nov. 2021. DOI: 10.1016/J.JIP.2020.107397.
4. Couch J. Free and Occluded Virus, similar to Baculovirus, in Hepatopancreas of Pink Shrimp. Nat 1974 2475438 1974; 247:229-231.
5. Couch J. An enzootic nuclear polyhedrosis virus of pink shrimp: ultrastructure, prevalence, and enhancement. J Invertebr Pathol 1974; 24:311-331.
6. OIE. Chapter 2.2.11. Tetrahedral Baculovirusis (Baculovirus Penaei). In: Manual of Diagnostic Tests for Aquatic Animals. Paris, France: World Organization for animal health. https://www.oie.int/fileadmin/Home/eng/Health_standards/aahm/current/chapitre_tetrahedral_baculovirosis.pdf (2019, accessed 23 Feb. 2022).
7. Bonami J R, Bruce L D, Poulos B T, Mari J, Lightner D V. Partial characterization and cloning of the genome of PvSNPV (=BP-type virus) pathogenic for Penaeus vannamei. Dis Aquat Organ 1995; 23:59-66.
8. Kamita S G, Kang K-D, Inceoglu A B, Hammock B D. Genetically Modified Baculoviruses for Pest Insect Control. Ref Modul Life Sci. Epub ahead of print 1 Jan. 2017. DOI: 10.1016/B978-O-12-809633-8.04074-7.
9. Tanada Y, Kaya H. Insect Pathology. San Diego: Academic Press. https://www.sciencedirect.com/book/9780123849847/insect-pathology (1993, accessed 17 Jan. 2022).
10. Szewczyk B, Hoyos-Carvajal L, Paluszek M, Skrzecz I, Lobo De Souza M. Baculoviruses-re-emerging biopesticides. Biotechnol Adv 2006; 24:143-160.
11. Assenberg R, Wan P T, Geisse S, Mayr L M. Advances in recombinant protein expression for use in pharmaceutical research. Curr Opin Struct Biol 2013; 23:393-402.
12. Chen C Y, Lin C Y, Chen G Y, Hu Y C. Baculovirus as a gene delivery vector: recent understandings of molecular alterations in transduced cells and latest applications. Biotechnol Adv 2011; 29:618-631.
13. Clem R J, Passarelli A L. Baculoviruses: Sophisticated Pathogens of Insects. PLoS Pathog; 9. Epub ahead of print November 2013. DOI: 10.1371/JOURNAL.PPAT.1003729.
14. Couch J A. The membranous labyrinth in baculovirus-infected crustacean cells: possible roles in viral reproduction. Dis Aquat Org 1989; 7:39-53.
15. Overstreet R M, Stuck K C, Krol R A, Hawkins W E. Experimental Infections with Baculovirus penaei in the White Shrimp Penaeus vannamei (Crustacea: Decapoda) as a Bioassay. J World Aquac Soc 1988; 19:175-187.
16. Hammer H S, Stuck K C, Overstreet R M. Infectivity and Pathogenicity of Baculovirus penaei(BP) in Cultured Larval and Postlarval Pacific White Shrimp, Penaeus vannamei, Related to the Stage of Viral Development. J Invertebr Pathol 1998; 72:38-43.
17. OIE. Manual of diagnostic test for aquatic animals. Paris, France: World Organization for animal health; 2021.
18. OIE. Chapter 2.2.10. Spherical Baculovirus (Penaeus monodon-type Baculovirus). In: Manual of Diagnostic Tests for Aquatic Animals. Paris, France: World Organization for animal health. https://www.oie.int/fileadmin/Home/eng/Health_standards/aahm/current/chapitre_spherical_baculovirus.pdf (2019, accessed 23 Feb. 2022).
19. Liu Y-M, Qiu L, Sheng A-Z, Wan X-Y, Cheng D-Y, et al. Quantitative detection method of Enterocytozoon hepatopenaei using TaqMan probe real-time PCR. J Invertebr Pathol 2018; 151:191-196.
20. Bolger A M, Lohse M, Usadel B. Trimmomatic: a flexible trimmer for Illumina sequence data. Bioinformatics 2014; 30:2114.
21. Seemann T. Genome analysis Prokka: rapid prokaryotic genome annotation. 2014; 30:2068-2069.
22. Bateman K S, Kerr R, Stentiford G D, Bean T P, Hooper C, et al. Identification and Full Characterisation of Two Novel Crustacean Infecting Members of the Family Nudiviridae Provides Support for Two Subfamilies. Viruses 2021, Vol 13, Page 1694 2021; 13:1694.
23. Bezier A, Theze J, Gavory F, Gaillard J, Poulain J, et al. The Genome of the Nucleopolyhedrosis-Causing Virus from Tipula oleracea Sheds New Light on the Nudiviridae Family. J Virol 2015; 89:3008.
24. Katoh K, Misawa K, Kuma K I, Miyata T. MAFFT: a novel method for rapid multiple sequence alignment based on fast Fourier transform. Nucleic Acids Res 2002; 30:3059-3066.
25. Katoh K, Standley D M. MAFFT Multiple Sequence Alignment Software Version 7: Improvements in Performance and Usability. Mol Biol Evol 2013; 30:772-780.
26. Kumar S, Stecher G, Li M, Knyaz C, Tamura K. MEGA X: Molecular Evolutionary Genetics Analysis across Computing Platforms. Mol Biol Evol 2018; 35:1547-1549.
27. Yang, Lee D, Wang Y, Hu J, Li W, et al. The genome and occlusion bodies of marine Penaeus monodon nudivirus (PmNV, also known as MBV and PemoNPV) suggest that it should be assigned to a new nudivirus genus that is distinct from the terrestrial nudiviruses. BMC Genomics; 15. Epub ahead of print 14 Jul. 2014. DOI: 10.1186/1471-2164-15-628.
28. Chiang P, Liao I C. THE PRACTICE OF GRASS PRAWN (Penaeus monodon) CULTURE IN TAIWAN FROM 1968 TO 19841. J World Maric Soc 1985; 16:297-315.
29. Sajjan D B, Hinchigeri S B. Structural Organization of Baculovirus Occlusion Bodies and Protective Role of Multilayered Polyhedron Envelope Protein. Food Environ Virol 2016; 8:86-100.
30. Wang Y, Jehle J. Nudiviruses and other large, double-stranded circular DNA viruses of invertebrates: new insights on an old topic. J Invertebr Pathol 2009; 101:187-193.
31. van Oers M, Vlak J. Baculovirus Genomics. Curr Drug Targets 2007; 8:1051-1068.
32. Harrison R L, Herniou E A, Bezier A, Jehle J A, Burand J P, et al. ICTV virus taxonomy profile: Nudiviridae. J Gen Virol 2020; 101:3-4.
33. Wang Y, Burand J, Jehle J. Nudivirus genomics: Diversity and classification. Virol Sin 2007 222 2008; 22:128-136.
34. Murphy F, Fauquet C, Bishop D, Garbrial S, Jarvis A, et al. Virus Taxonomy. Sixth Report of the International Committee on Taxonomy of Viruses. Springer. https://link.springer.com/content/pdf/10.1007%2F978-3-7091-6607-9.pdf (1995, accessed 6 Jan. 2022).
35. Wallace M A, Coffman K A, Gilbert C, Ravindran S, Albery G F, et al. The discovery, distribution, and diversity of DNA viruses associated with Drosophila melanogaster in Europe. Virus Evol; 7. Epub ahead of print 1 Jan. 2021. DOI: 10.1093/VE/VEAB031.
36. Wang Y, Kleespies R, Ramle M, Jehle J. Sequencing of the large dsDNA genome of Oryctes rhinoceros nudivirus using multiple displacement amplification of nanogram amounts of virus DNA. J Virol Methods 2008; 152:106-108.
37. Cruz-Flores R, Mai H N, Noble B L, Schofield P J, Dhar A K. Detection of Enterocytozoon hepatopenaei using an invasive but non-lethal sampling method in shrimp (Penaeus vannamei). J Microbiol Methods 2019; 162:38-41.
38. Liu S, Sappington T W, Coates B S, Bonning B C. Nudivirus Sequences Identified from the Southern and Western Corn Rootworms (Coleoptera: Chrysomelidae). Viruses; 13. Epub ahead of print 1 Feb. 2021. DOI: 10.3390/V13020269.
39. Holt C C, Stone M, Bass D, Bateman K S, van Aerle R, et al. The first clawed lobster virus Homarus gammarus nudivirus (HgNV n. sp.) expands the diversity of the Nudiviridae. Sci Reports 2019 91 2019; 9:1-15.
40. Allain T W, Stentiford G D, Bass D, Behringer D C, Bojko J. A novel nudivirus infecting the invasive demon shrimp Dikerogammarus haemobaphes (Amphipoda). Sci Rep; 10. Epub ahead of print 1 Dec. 2020. DOI: 10.1038/S41598-020-71776-3.
41. Farrell P, Swevers L, Iatrou K. Insect Cell Culture and Recombinant Protein Expression systems. Comprehens. Elsevier. https://reader.elsevier.com/reader/sd/pii/B0444519246000636?token=186F29C9615B2D2C4 42F3AD6F7CA93A51B21807E64611E7A5C451F751DD5557D6E89CF53B3365EFBA662 69CBFC0430ED&originRegion=us-east-1&originCreation=20220115201247 (2005, accessed 15 Jan. 2022).
42. Xing K, Deng R, Wang J, Feng J, Huang M, et al. Analysis and prediction of baculovirus promoter sequences. Virus Res 2005; 113:64-71.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

Further attributes, features, and embodiments of the present invention can be understood by reference to the following numbered aspects of the disclosed invention. Reference to disclosure in any of the preceding aspects is applicable to any preceding numbered aspect and to any combination of any number of preceding aspects, as recognized by appropriate antecedent disclosure in any combination of preceding aspects that can be made. The following numbered aspects are provided:
1. A nudiviral nucleic acid corresponding to a polyhedrin gene promoter of a nudivirus, a variant thereof, or a derivative thereof.
2. The nudiviral nucleic acid of aspect 1, wherein the nudivirus is an alpha nudivirus, betanudivirus, deltanudavirus, gammanudavirus, or an epsilonnudavirus.
3. The nudiviral nucleic acid of any one of aspects 1-2, wherein the nudivirus is a shrimp, fish, or insect nudivirus.
4. The nudiviral nucleic acid of any one of aspects 1-3, wherein the nucleic acid comprises or consists of a sequence that is 80-100% identical to SEQ ID NO: 5, 6, 7, or 8.
5. The nudiviral nucleic acid of any one of aspects 1-4, wherein the nucleic acid comprises or consists of a sequence that is 80-100% identical to SEQ ID NO: 7.
6. An engineered polynucleotide comprising:
   the nudiviral nucleic acid of any one of aspects 1-5; and
   a polynucleotide encoding a non-polyhedrin polynucleotide and/or polypeptide, wherein the nudiviral nucleic acid is operatively coupled to the polynucleotide encoding a non-polyhedrin polynucleotide.
7. A vector or vector system comprising:
(a) a nudiviral nucleic acid of any one of aspects 1-5, (b) an engineered polynucleotide of aspect 6, or both (a) and (b).
8. The vector of aspect 7, wherein the vector is an expression vector or vector system.
9. A cell comprising: (a) a nudiviral nucleic acid of any one of aspects 1-5; (b) an engineered polynucleotide of aspect 6; (c) a vector or vector system of aspect 7 or aspect 8; or (d) any combination of (a)-(c).
10. The cell of aspect 9, wherein the cell is a prokaryotic or eukaryotic cell.
11. The cell of any one of aspects 9-10, wherein the cell is a shrimp cell, fish cell, insect cell, or a plant cell.
12 The cell of any one of aspects 9-10, wherein the cell is a mammalian cell, optionally a human cell.
13. A cell population comprising: one or more cells as in any one of aspects 9-12.
14. An organism comprising: (a) a nudiviral nucleic acid of any one of aspects 1-5; (b) an engineered polynucleotide of aspect 6; (c) a vector or vector system of aspect 7 or aspect 8; (d) a cell or cell population as in any one of aspects 9-13; or (e) any combination of (a)-(d).
15. The organism of aspect 14, wherein the organism is a non-human animal, insect, or a plant.
16. The organism of aspect 14, wherein the organism is a crustacean or fish.
17. A formulation comprising: (a) a nudiviral nucleic acid of any one of aspects 1-5; (b) an engineered polynucleotide of aspect 6; (c) a vector or vector system of aspect 7 or aspect 8; (d) a cell or cell population as in any one of aspects 9-13; or (e) any combination of (a)-(d); and a pharmaceutically acceptable carrier.
18. A kit comprising: (a) a nudiviral nucleic acid of any one of aspects 1-5; (b) an engineered polynucleotide of aspect 6; (c) a vector or vector system of aspect 7 or aspect 8; (d) a cell or cell population as in any one of aspects 9-13; (e) a formulation of aspect 17; or (f) any combination of (a)-(e).
19. A method comprising: expressing an engineered polynucleotide as in aspect 6 or a vector or vector system as in any one of aspects 7-8 in vitro, in vivo, or ex vivo.
20. A method of expressing an engineered nucleic acid, the method comprising: placing an engineered polynucleotide of aspect 6 or a vector or vector system of aspect 7 or aspect 8 under condition(s) and/or environment(s) such that the non-polyhedrin polynucleotide is transcribed and optionally translated.
21. The method of aspect 20, wherein expression occurs in vitro, in vivo, or ex vivo.
22. The method of any one of aspects 20-21, further comprising operatively coupling the non-polyhedrin encoding nucleic acid to a nudiviral nucleic acid of any one of aspects 1-5.

What is claimed is:

1. A nudiviral nucleic acid corresponding to a polyhedrin gene promoter of a nudivirus, a variant thereof, or a derivative thereof.

2. The nudiviral nucleic acid of claim 1, wherein the nudivirus is an alpha nudivirus, betanudivirus, deltanudavirus, gammanudavirus, or an epsilonnudavirus.

3. The nudiviral nucleic acid of claim 1, wherein the nudivirus is a shrimp, fish, or insect nudivirus.

4. The nudiviral nucleic acid of claim 1, wherein the nucleic acid comprises or consists of a sequence that is 80-100% identical to SEQ ID NOs: SEQ ID NO: 5, 6, 7, or 8.

5. The nudiviral nucleic acid of claim 1, wherein the nucleic acid comprises a sequence that is 100% identical to SEQ ID NO: 7.

6. An engineered polynucleotide comprising:
the nudiviral nucleic acid of claim 1; and
a polynucleotide encoding a non-polyhedrin polynucleotide and/or polypeptide, wherein the nudiviral nucleic acid is operatively coupled to the polynucleotide encoding a non-polyhedrin polynucleotide.

7. A vector or vector system comprising:
(a) a nudiviral nucleic acid of claim 1, (b) an engineered polynucleotide comprising the nudiviral nucleic acid and a polynucleotide encoding a non-polyhedrin polynucleotide and/or polypeptide, wherein the nudiviral nucleic acid is operatively coupled to the polynucleotide encoding a non-polyhedrin polynucleotide, or both (a) and (b).

8. The vector of claim 7, wherein the vector is an expression vector or vector system.

9. A cell comprising:
a. a nudiviral nucleic acid of claim 1;
b. an engineered polynucleotide comprising the nudiviral nucleic acid of (a) and a polynucleotide encoding a non-polyhedrin polynucleotide and/or polypeptide, wherein the nudiviral nucleic acid is operatively coupled to the polynucleotide encoding a non-polyhedrin polynucleotide;
c. a vector or vector system comprising (a) and/or (b); or
d. any of (a)-(c).

10. The cell of claim 9, wherein the cell is a prokaryotic or eukaryotic cell.

11. The cell of claim 9, wherein the cell is a shrimp cell, fish cell, insect cell, or a plant cell.

12. The cell of claim 9, wherein the cell is a mammalian cell, optionally a human cell.

13. A cell population comprising:
one or more cells as in claim 9.

14. An organism comprising:
a. a nudiviral nucleic acid of claim 1;
b. an engineered polynucleotide comprising the nudiviral nucleic acid of (a) and a polynucleotide encoding a non-polyhedrin polynucleotide and/or polypeptide, wherein the nudiviral nucleic acid is operatively coupled to the polynucleotide encoding a non-polyhedrin polynucleotide;
c. a vector or vector system comprising (a) and/or (b;
d. a cell or cell population comprising (a), (b), and/or (c); or
e. any combination thereof.

15. The organism of claim 14, wherein the organism is a non-human animal, insect, or a plant.

16. The organism of claim 14, wherein the organism is a crustacean or fish.

17. A formulation comprising:
a. a nudiviral nucleic acid of claim 1;
b. an engineered polynucleotide comprising the nudiviral nucleic acid of (a) and a polynucleotide encoding a non-polyhedrin polynucleotide and/or polypeptide, wherein the nudiviral nucleic acid is operatively coupled to the polynucleotide encoding a non-polyhedrin polynucleotide;
c. a vector or vector system comprising (a) and/or (b;
d. a cell or cell population comprising (a), (b), and/or (c); or
e. any combination (a)-(d); and
a pharmaceutically acceptable carrier.

18. A kit comprising:
a. a nudiviral nucleic acid of claim 1;
b. an engineered polynucleotide comprising the nudiviral nucleic acid of (a) and a polynucleotide encoding a non-polyhedrin polynucleotide and/or polypeptide, wherein the nudiviral nucleic acid is operatively coupled to the polynucleotide encoding a non-polyhedrin polynucleotide;
c. a vector or vector system comprising (a) and/or (b;
d. a cell or cell population comprising (a), (b), and/or (c); or;
e. a formulation comprising (a), (b), (c), (d), or any combination thereof, and a pharmaceutically acceptable carrier; or
f. any combination of (a)-(e).

19. A method comprising:
expressing an engineered polynucleotide as in claim 6 or a vector or vector system comprising the engineered polynucleotide in vitro, in vivo, or ex vivo.

20. A method of expressing an engineered nucleic acid, the method comprising:
placing an engineered polynucleotide of claim 6 or a vector or vector system comprising the engineered polynucleotide under condition(s) and/or environment(s) such that the non-polyhedrin polynucleotide is transcribed and optionally translated.

21. The method of claim 20, wherein expression occurs in vitro, in vivo, or ex vivo.

22. The method of claim 20, further comprising operatively coupling the non-polyhedrin encoding nucleic acid to a nudiviral nucleic acid corresponding to a polyhedrin gene promoter of a nudivirus, a variant thereof, or a derivative thereof.

23. The method of claim 22, wherein the nudivirus is an alpha nudivirus, betanudivirus, deltanudavirus, gammanudavirus, or an epsilonnudavirus.

24. The method of claim 22, wherein the nudivirus is a shrimp, fish, or insect nudivirus.

25. The method of claim 22, wherein the nucleic acid comprises or consists of a sequence that is 80-100% identical to SEQ ID NOs: SEQ ID NO: 5, 6, 7, or 8.

26. The method of claim 22, wherein the nucleic acid comprises a sequence that is 100% identical to SEQ ID NO: 7.
